SEQUENCE LISTING (32 sequences)

SEQ ID NO: 1 - DNA, 20 bp, Artificial sequence
  Primer for outer 5′PCR
acacactgctcccaggatga

SEQ ID NO: 2 - DNA, 24 bp, Artificial sequence
  Primer for inner 5′PCR
agccaagatgactgtcgtgcattc

SEQ ID NO: 3 - DNA, 22 bp, Artificial sequence
  Forward primer
ccatggacaagttttggtggca

SEQ ID NO: 4 - DNA, 20 bp, Artificial sequence
  Reverse primer
gtatatcccctgggaaggct

SEQ ID NO: 5 - DNA, 22 bp, Artificial sequence
  Reverse primer
cagctgcttcttgagcttggga

SEQ ID NO: 6 - DNA, 20 bp, Artificial sequence
  Reverse primer
aagcaggcttggacaaggtt

SEQ ID NO: 7 - DNA, 24 bp, Artificial sequence
  Reverse primer
ctcgttcaacagagagacaacagc

SEQ ID NO: 8 - DNA, 20 bp, Artificial sequence
  Forward primer
gaagaaagccagtgcgtctc

SEQ ID NO: 9 - DNA, 23 bp, Artificial sequence
  Reverse primer
gagggctgtccttaacgcctagc

SEQ ID NO: 10 - DNA, 22 bp, Artificial sequence
  Primer for outer 3′PCR
ttgaggcgctaaagggcttacc

SEQ ID NO: 11 - DNA, 23 bp, Artificial sequence
  Primer for inner 3′PCR
cagaccatggcatctacggaagg

SEQ ID NO: 12 - DNA, 22 bp, Artificial sequence
  Forward primer
ccacccagaagactgtggatgg

SEQ ID NO: 13 - DNA, 20 bp, Artificial sequence
  Reverse primer
cactgacacgttggcagtgg

SEQ ID NO: 14 - DNA, 20 bp, Artificial sequence
  Forward primer
tataacctgccgctttgcag

SEQ ID NO: 15 - DNA, 20 bp, Artificial sequence
  Reverse primer
gcaggtctcaaatccgatgc

SEQ ID NO: 16 - DNA, 20 bp, Artificial sequence
  Forward primer
gcccgttgaggcgctaaagg

SEQ ID NO: 17 - DNA, 20 bp, Artificial sequence
  Reverse primer
agcactatccggcagctgtg

SEQ ID NO: 18 - DNA, 20 bp, Artificial sequence
  Forward primer
ttcggtccgccatcctcgtc

SEQ ID NO: 19 - DNA, 20 bp, Artificial sequence
  Reverse primer
cacttccacctgcttgggca

SEQ ID NO: 20 - protein, 65 aa, Homo sapiens
MDKFWWHAAWGLCLVPLSLAQIVPTICPSRWKCECTTVILAQRNPSTGTWACACVTSWGRICCSP

SEQ ID NO: 21 - protein, 565 aa, Homo sapiens
MPKQVEVRMHDSHLGSEEPKHRHLGLRLCDKLGKNLLLTLTVFGVILGAVCGGLLRLASPIHPDVVMLIAFPGDILMRMLKMLILPLIISSLITGLSGLDAKASGRLGTRAMVYYMSTTIIAAVLGVILVLAIHPGNPKLKKQLGPGKKNDEVSSLDAFLDLIRNLFPENLVQACFQQIQTVTKKVLVAPPPDEEANATSAVVSLLNETVTEVPEETKMVIKKGLEFKDGMNVLGLIGFFIAFGIAMGKMGDQAKLMVDFFNILNEIVMKLVIMIMWYSPLGIACLICGKIIAIKDLEVVARQLGMYMVTVIIGLIIHGGIFLPLIYFVVTRKNPFSFFAGIFQAWITALGTASSAGTLPVTFRCLEENLGIDKRVTRFVLPVGATINMDGTALYEAVAAIFIAQMNGVVLDGGQIVTVSLTATLASVGAASIPSAGLVTMLLILTAVGLPTEDISLLVAVDWLLDRMRTSVNVVGDSFGAGIVYHLSKSELDTIDSQHRVHEDIEMTKTQSIYDDMKNHRESNSNQCVYAAHNSVIVDECKVTLAANGKSADCSVEEEPWKREK

SEQ ID NO: 22 - DNA, 5748 bp, Homo sapiens
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccactttgatgagcactagtgctacagcaactgagacagcaaccaagaggcaagaaacctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacagaaaaaaaaaaaaaaaaa

SEQ ID NO: 23 - DNA, 1905 bp, Homo sapiens
attgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgtgccaacaatatgcccaagcaggtggaagtgcgaatgcacgacagtcatcttggctcagaggaacccaagcaccggcacctgggcctgcgcctgtgtgacaagctggggaagaatctgctgctcaccctgacggtgtttggtgtcatcctgggagcagtgtgtggagggcttcttcgcttggcatctcccatccaccctgatgtggttatgttaatagccttcccaggggatatactcatgaggatgctaaaaatgctcattctccctctaatcatctccagcttaatcacagggttgtcaggcctggatgctaaggctagtggccgcttgggcacgagagccatggtgtattacatgtccacgaccatcattgctgcagtactgggggtcattctggtcttggctatccatccaggcaatcccaagctcaagaagcagctggggcctgggaagaagaatgatgaagtgtccagcctggatgccttcctggaccttattcgaaatctcttccctgaaaaccttgtccaagcctgctttcaacagattcaaacagtgacgaagaaagtcctggttgcaccaccgccggacgaggaggccaacgcaaccagcgctgttgtctctctgttgaacgagactgtgactgaggtgccggaggagactaagatggttatcaagaagggcctggagttcaaggatgggatgaacgtcttaggtctgatagggtttttcattgcttttggcatcgctatggggaagatgggagatcaggccaagctgatggtggatttcttcaacattttgaatgagattgtaatgaagttagtgatcatgatcatgtggtactctcccctgggtatcgcctgcctgatctgtggaaagatcattgcaatcaaggacttagaagtggttgctaggcaactggggatgtacatggtaacagtgatcataggcctcatcatccacgggggcatctttctccccttgatttactttgtagtgaccaggaaaaaccccttctccttttttgctggcattttccaagcttggatcactgccctgggcaccgcttccagtgctggaactttgcctgtcacctttcgttgcctggaagaaaatctggggattgataagcgtgtgactagattcgtccttcctgttggagcaaccattaacatggatggtacagccctttatgaagcggtagccgccatctttatagcccaaatgaatggtgttgtcctggatggaggacagattgtgactgtaagcctcacagccaccctggcaagcgtcggcgcggccagtatccccagtgccgggctggtcaccatgctcctcattctgacagccgtgggcctgccaacagaggacatcagcctgctggtggctgtggactggctgctggacaggatgagaacttcagtcaatgttgtgggtgactcttttggggctgggatagtctatcacctctccaagtctgagctggataccattgactcccagcatcgagtgcatgaagatattgaaatgaccaagactcaatccatttatgatgacatgaagaaccacagggaaagcaactctaatcaatgtgtctatgctgcacacaactctgtcatagtagatgaatgcaaggtaactctggcagccaatggaaagtcagccgactgcagtgttgaggaagaaccttggaaacgtgagaaataa

SEQ ID NO: 24 - DNA, 12021 bp, Homo sapiens
gggtgatgtcagctctcgacgaaaatagagagggatcgcctgcaaatccccagctccggcggggctaaaccttgcaatccctccctggccggcgccgagccagagcgcagcggcctccaccgcctccccaggcgcgcacacacccgcacacgcgcacgcacgctcaccgtcctctgccaccactctctgctcccgccactcgccgcgcccgcgagccccgcagcaaagcacaggtggcagcggctgcaggggcgcatcgccggcgtgcgccctcctgcagccctgggcgcatcgctctctcggggaagccaccctcggagcccccggagctccccgccaagcgccatccccgcgggcggaggggagcgcgggtcgcgcgccgtggagagccgggacgcggattagcgcccgcaggagcctcctgcgcccgttgaggcgctaaagggcttaccccggaggcgggtggaagggcgggcagaggctcctcttaaataccgctcccggccgcacttcgcgctcaccccggcgtccgctttctccctcgcccacagctgccggatagtgctgaagaggagggggcgttccccagaccatggcatctacggaaggtgccaacaatatgcccaagcaggtggaagtgcgaatgcacgacagtcatcttggctcagaggaacccaagcaccggcacctgggcctgcgcctgtgtgacaagctggggaagaatctgctgctcaccctgacggtgtttggtgtcatcctgggagcagtgtgtggagggcttcttcgcttggcatctcccatccaccctgatgtggttatgttaatagccttcccaggggatatactcatgaggatgctaaaaatgctcattctccctctaatcatctccagcttaatcacagggttgtcaggcctggatgctaaggctagtggccgcttgggcacgagagccatggtgtattacatgtccacgaccatcattgctgcagtactgggggtcattctggtcttggctatccatccaggcaatcccaagctcaagaagcagctggggcctgggaagaagaatgatgaagtgtccagcctggatgccttcctggaccttattcgaaatctcttccctgaaaaccttgtccaagcctgctttcaacagattcaaacagtgacgaagaaagtcctggttgcaccaccgccggacgaggaggccaacgcaaccagcgctgttgtctctctgttgaacgagactgtgactgaggtgccggaggagactaagatggttatcaagaagggcctggagttcaaggatgggatgaacgtcttaggtctgatagggtttttcattgcttttggcatcgctatggggaagatgggagatcaggccaagctgatggtggatttcttcaacattttgaatgagattgtaatgaagttagtgatcatgatcatgtggtactctcccctgggtatcgcctgcctgatctgtggaaagatcattgcaatcaaggacttagaagtggttgctaggcaactggggatgtacatggtaacagtgatcataggcctcatcatccacgggggcatctttctccccttgatttactttgtagtgaccaggaaaaaccccttctccttttttgctggcattttccaagcttggatcactgccctgggcaccgcttccagtgctggaactttgcctgtcacctttcgttgcctggaagaaaatctggggattgataagcgtgtgactagattcgtccttcctgttggagcaaccattaacatggatggtacagccctttatgaagcggtagccgccatctttatagcccaaatgaatggtgttgtcctggatggaggacagattgtgactgtaagcctcacagccaccctggcaagcgtcggcgcggccagtatccccagtgccgggctggtcaccatgctcctcattctgacagccgtgggcctgccaacagaggacatcagcctgctggtggctgtggactggctgctggacaggatgagaacttcagtcaatgttgtgggtgactcttttggggctgggatagtctatcacctctccaagtctgagctggataccattgactcccagcatcgagtgcatgaagatattgaaatgaccaagactcaatccatttatgatgacatgaagaaccacagggaaagcaactctaatcaatgtgtctatgctgcacacaactctgtcatagtagatgaatgcaaggtaactctggcagccaatggaaagtcagccgactgcagtgttgaggaagaaccttggaaacgtgagaaataaggatatgagtctcagcaaattcttgaataaactccccagcgtatcctatggtaactgatgatataaacaagctttctttaaaaaggaaaaaaatgcgtatatttctatgtttacttaatctgttagccgaggcttagaggagctcttgtgagtcagtgatgacaggcacggtgctgtgtctttgccaaataatgctttataaccgtctaattttctcacttgtattattatttgaatggatgctgctggaggaatcagttggaattgaagacacgttcttgccagcttcccttttctcccaagatgcagaaatgtggatgctcttttcccaggggacatgagtaaagcagtgtggtacactccagggacttgggaaaatgagcaaacacacagcgtgttattccttaaagtgttctccatgtctcgccttgttatgcacaagagattctattaaaagcctctagaagtaactccccttaaaatgtctagtaaagcttgcacatggattgattaaaagcaaatacctgtcttagggaattctgacaatttatcttccatatgctctttaagtaaaatgtttcaaagacagttttaaagggagccatgctcttaaaggcagttgattaaagaacctgttacatctctgccttaccctgtgtaatctgtgagaacaatggttgaaatttcaaagtatgtttcattattctttctaaatttgataattgattaggaagtattttatatataaccactgtagatattgacaaaagtaagagagcacagtcaacataaagtttaaccagagttaaatattcaaatttatttatgttcggtttgccttcacctggtgtagtaaaatcaaatgagattatttggtatgtgttttgctttgtttaacccaaaagattattttttggttcctaaagaaaaagatatttttaatctgtcaattatttcagtcatcatctcatcgtcctagaaagcctggtcattttactgtcctcacacatggaagacacttgtgttggctgtgcctcttttaatgtcatctcttgatgaaacaaaaatactggtatttatccaatatgttgaaagcgttttgttatcttctatcaaagcacataaaaccagttataggacacagttggaagcagagccagtcttcctcctcagtcttctcagtagaaaggaacagaaaacaaacatctgtgcaaaaccctataaaagtgatattcctatggcagagtccaggcagccttgaacaatgacatggcaacaaaggatggctctgtacaacttcacagatggaatcttgtttaaggctgtgaagttttaaggaataaataatataattggtgtagcacagtgccttgcatgcagtaggcagccaacaaatccttaatgattggtaatgcaagctaaaatttttaatgtctctactatccttttttattgaataacgcacagtattttcctaaggttgctgggtttgggagtaagcttgggacttgaatcattgagaaataaagcacatttgactctaagaagtatgtagaaccttgtttataggtgagaataggctgtattatatgaaaccagcggagggatgctgagacagtcatggtttatagaatccaaattctctgagatgagggtgcttatggacctcaaatagcctttcttatctgctccttgtgacttctctcttttaaatcagatacctttgaaacatacaagagacatgagattccagttgtttttgtctcctaaaccctagtgatttttggataaggatcactactgaacagtattaggcaacttttcatttttgcttttcaaatgacattggtctttatttccagtagatgggcagtaaacagggtcagggtggtcaagggatatgattaagtgtagtagtagaaaatgcattctttttttttttttttttttagacagagtcttgctctgttgctcaggctggagtacagtggcacgatctcagctcactgcactgcaatgtccacctctctagttcaagcgattctcctgtctcagcctcccgagtagctgggactacaggcacatgccaccatgcctggctaattttttgtatttttagtacagatgggtttcattatgttagccaggatggtctcgatctcctgacctcatgatccacccgccatggcctcccaaagtgctgggattacaggcgtgagccaccgtgcccggccagaaaatacattctttacagcaaatgttataggttaaggagacagttcctgggtttagctgtgaaactgtttaggattttctttaagtggtgttcccaagagcttttttctggtaagttggaggctataggcaaagcgtctaggagcttgttgttttttgctatggaaggccagctttgggatctatatacatgggcatgtttctcaatggagtggtcacaggattctcttctcagaactctgcctgaaacctttccggtgtgttttgtcttcaactttttcctagctatcctattagcctgtaaatatgtcagcagacaatgtgtttggaagaaacaaggtgtacaacttcacaaagcaagttttttctggtctgagacttactgtgtgcaggaagtctcgtgagaatatacttagaaaatcacacttatctggaaattcaataagtcatattaaaaatatacaatttcttgaactcaggactagcctgaaaatccaaaatgcatgtagagttgacatgttgacatatgtctatgtcctctgtttttatttaagcctcatttatttctcccttgccatgcccatttgggattttctttgcccagcatatgtgccataaacagaacaaatggatccagggtatacctctaataacctttatctaaatagaatacatttaacctgtcaattatatgtatagatttatcctaattggttcaacatatatatgaatgcttgtaatagtctgttactatttgataatgcataaatgttatatttatatacagttacagcaggccagaaacatacatacactgcatcctgtaaggtctgtaaatcagggcttcggcaatgccattttgctatagactcattcatgttttttaggaatcagaatcatttcaggagaaaaccaactaagtagactttaattcttctctttctcattcttgccctttgagaaacccaagtggaatcatcacttaattacttactcaaattcaatgaatcaaatccaatccaatcagtgagaagaatagcttatctttcaaggtaatactttaccttagatctttgagggcatgcttcccatacaaagagcatcagaaaatcagggcaatcatctagatctcacagatcttcaacataggactctaggttatgagagctgaaccacatagctatcagtttcagtttttacttcctattgagtgtgtagaaacacagcctatggttttgctgtctgcggaagttaccataccctgtaaaagacaaatatttctttctcctctcgaaccactagtcagcctggatattcatgaaagctgaaagaaggggtgccaaaatgcctagctcctggatttagactcatggaatatgagaaaggcttagggaaaattcaagtcatggggagattcctgctaggtgatgccaggaatctcaatgacctgttgattagagggtctctggtcatttccacttgtgtgtggagaaatacatcatttgcatatgtatctagtcaagaagcccttttggagcaaagtggttgggaaactcatttactgggtccaccaaaggaaacccaattctgccatgtttttgcaaatactacacgtccctttgctctcaatagcaaaagtgatgttatagttttttttacttttgtcagaatcaaggcttcggccacctgtgcaatattttcccatggaatgcagcagtaaatccaagtaggtatatccgtgctcagagagaattctccatagttctgactcctgaaaaacaagtctcattagctctcatctagtttgatgactaaaagtgcacagcagttataccccagtggaagtgatttttctcacctagctgctctctaactcttacggctatctagttgtctgagctgctggtttgttttacttattctccaactgaactcattccaaaccaagaaaaaggaccaaatgaaattttcagttattatcatacttgctccttgaataagaatgctagagtctggaggcagaagaagactaagttaaaaaaaaaacagcctgaaggaattctggagtgagttttgtaagtctaattgtcaaagaagataaatggtctcagtatctgtagtaatttctggttaagcctagagggatcactgagtgccaatccaagcaggtggcactagaggcagattgttctcctagcatagagtgagtttccagctgctcatgacacattttcttatgattttccctaaaagaaaaccaaattttctactccacctcttcctttcctatcattccacttattgttgctgctttgctaacagccagcttagtaagccatcacacaagttagtgtgaggcttggttttaaaatagataacagagaatcttgattaaatagtatccaactttgtagcataacacagctaattcaggcaatgacatagagatgataagaaacaacatggtttggtagagggaacatttgatttagactctgcccatttttagctgtatgacttacataagtcattttgtgtccaagcctcattttctcccatatgaaaagtgaaggggttggattaaatgactaaaatccccttccagccctatgagcccaatgtattatgatctctgctttgtttccttcttaagaggcttcctactataaaatgtgacctatttacattttaagttgaagtagcccacaataatgaataatcaatttagatttttctcatctcctttgggagaaattaaattcaagcctctattcatttgatgttttacaacaagcttcaaagttggccatgttcattcacagtttgatattttgagacaccaataaaagttttttaataaaagttcccttgacttaaactcaccttcctaatagaagtagtgatttggctcacggaaaaatgtttccagagtcaacatgagaggactggatgaacctacagcctcactcaagctgttgcatcattgctgctgtagcaacagagccctctctgaatacccaaaacacacaattttctccaacaattctaattgcccacccagtatgggaatttgagagcctttggtgaataaccttgatcactcttttgaaatttggtacaacacttgcacaatcacggcaactgtttgtaaatatcttttcttctccgccagtatgtagacatgcacacccattcacaagtaagcccgcaaattcattttcacattttcctcttggtttgtttttatcatgggtatcacattgcaggaagagatgctctaacttacaagaatggaattttctgactatcattgccctcttcaaggcctgattagtttgtagggtggatgggatacaatgcatggtctacaaggtccatgtcaccagccatgatctttaaacaacctcttgcactttctggcactgctgcaatggcccatggtggacacctctgctgtttgtccaaacttctgctaaatagagtagtgttggtatttgttgtgttgtgaactcctttgttgtcagagaagacttcagctcttcaggtttgcaagggcaagatgtaaagctaggactgaaaggattgggttttgtttttcggccggcttttttccattagtgcaaaaaacctgatgcaagggcagcaagataaacacaatttcttcattgacccagataagaacctgtagaaaagtgagagatgcccctaagttcctatttcatttatcctaaataatatcctgaataactactgatcagtttttttctatgtccagccttgcttcactttccaacctccaggccaaatattcattttgatattttacaaaatccttctggtttttacacagggcccttttgtgctgagaataaaaccagctattggaacagacctcactgctttgtagagtcagaagttgacctagagaatgggtagcaacaggtcctcagtttctctggagacacccccaatccttgggtgagtgttgaaacctgacaactgactgttagccttgtcatcatcctcactggagatggagatgttgcagtcggagcctcactccttctggggcactgtttcttctcatggcaaccattttgcctgcctggaaacagcctgttgcctttgctagggggcaaatacacacactagccaaggaagttggtccagcacatctctgaggtttctcccccatggacccatggagcgaattccctgagataaatgatcagaaagttggctgtctggtggtttaatactccataagagttacttcccttatagaaagtcatcctttggcatctagatttttctacaaatcaccttaactccctccctttctctttctcctggaaaacctcagcattgcatctccatgttgcacaacacagatcagattatctggtcactatagagatttgtatataaaaaaactacttttttgaggattttgtatattgtttttctatttgttttctacagcttgaggaaagagctggcaaactgtgaatctaacttgatcttgttgccagcagatatattttggcttcctggtaagagtctgtgttaccagggacaatatattgccctctgatcagtattgcaccctaaatccacaaattcctccagcccagtcttacatttttttaaacaaatcccctagcttgtttgctattatttataaagagcattagaaaatgtatttatagacctggatgttatatgtttaatattaatttagcctttaataatgttataggtttgtaactattcttcagaaattataaagaactcagtgtagactgaattaatctatttagtatctgtaattttgcagacatattttcttacagtattttctatgtaaccacacatgtagaattataactaattagagcacaagaagtttcttcagcaatttagagctaccaattgtttcttgactatatagtcacatttgacaaatttaagaacccagtctttagtgatgcaataaacaaaatgaaccattaagaacaaggaattgcttaaatcccttagctggtgaggatatacatctaaataattcatctttctaactcaaggaatggtgctgattttttaaatgtttgacaccaggccttgtttttccagctgagcattctcattttgcttttctctaagactatcaaagacaaggtattaatagtaggattattcctagatcagaatgtttcatacattcctaaaggtttatgtggaaattggcttaggaaaactttgagtagcagagactgaggatgagtgctagagatgaaatcaggacagatttgttgcagttaattcttgccaagcaaattagtggtaaatgtcacattgttatgtgaattgagcacatatttttaaagaaagtttacaaaaaatttttagaaccaactatgaggcaatactgtatcactgggggctgggagtgggggcttagaatcatactgaaattgtttaaaagcagcccaggtagtttctgtcctcaggtgaataacagactatataacttccccgaaaggtaaaacgatagccactgcagagacgaggtgtcttccttccaccaaatactttctgagatggtggtaggagtagtatttttaccgtggtttaaaaagtagtcagttacatagaaagtgtattggtatgtatttgaacctgctcttcattaaacaaacagattagatgtaccctcgactggcaataattgtatctattttcaagtacagctagctgtcaaagcatgaagctcttgtgtatacacactgacacttggttcacgcatgaagaacagtgcctatgcactttgtgtagctataatgtaggtatctaggtgtaatttcagtgaaatggtgtatagatgtattgtaatttaaatgtatatgttatttttggctattcatctaaatgcagtagacatgttgatcggtgttttgcaaacatttctttttccttcttagataactagtcgtgaatcatttctcctctttctcagtgtggcttgggaatatatatgagtgaagaatttatctgtgaatcctttgtactgatgattgtttgaaagtctgtgtgtgtccagcacctttgtaaatacacaattcagagcagggatgggctgggtgtgtgtcctggttcttagtgaaaggtcatctcatgtctgtttaatacatggtgaatgcaactgtggaacttttgattacctagacttaggtaggtttagaatgagaacatccatctacagtcctcctttgcttggtggattgggctcagaggaacaaaaagttagtctgactctgtgcatattagcatcatgtctttagagaaaggtcagcctctctggttgccaaatactcatcatgatgctcatgacttaaaggttctgaggagccttgtctcccttggatttttgagtcagggtacaggaaaaaacattgctgactaactaactgcaaatgcatctgcaggtgaaaccctacgaaagcacagttctggctataaacttcagagttctctgtaaaaaacttagagcactagaagcacaggaatagtgagtgtacagcttatgcggttgtagaggggcaactgatgaacacaggtcccacatatatgagggagtatgacgttctctacctaatatgttctgtgtgcatgttttgaatgattgaagatgggattaactaatgcaagtttacagttgcctcctaaaacacacattctgtataattatcgctaaatacaatgctgtgaggtctatagttcctgtaacccctttctcctccccaaggacagagaagaactagccatgtgctatagggaaccctgagtgccctactcttttcccaagaagggtaaagcctacaatatcatcagggggcatgaagcacattaatttgcagtggctgcttcatatgaggagatatggtggacaggctaatttttccttgaaaatgtggcttcttcaactcctttcaaatttaggatggaatacttcctgaaataaaactgggctttatgcaggattctctttgaaaattcttgtatgtccagaacaaaagataaaactaattgtattcctcacattcacaatccccattggtctgaagtcacgtagcacagagcatctatagcacatagtgtttaaagactaatgaatgcaaaaagataaaatcttcaactaatttttgaattgtttctcatatatgctactagaaaatgccttgttgatgaagcacattttgggtagttgaggtcttttgttttcgcctttagctttctaagctttcttacaatgtggactgattactgtaacatttcacgtgtaaaataactggatattctttatatactggaaataacctgtgaatccaatatttcactaagtgttttaacttttgtgtatatatctctcatcaataaatgtggatttcaatttaaaaaaaaaaaaaaaa

SEQ ID NO: 25 - protein, 742 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 26 - protein, 574 aa, Homo sapiens
MASTEGANNMPKQVEVRMHDSHLGSEEPKHRHLGLRLCDKLGKNLLLTLTVFGVILGAVCGGLLRLASPIHPDVVMLIAFPGDILMRMLKMLILPLIISSLITGLSGLDAKASGRLGTRAMVYYMSTTIIAAVLGVILVLAIHPGNPKLKKQLGPGKKNDEVSSLDAFLDLIRNLFPENLVQACFQQIQTVTKKVLVAPPPDEEANATSAVVSLLNETVTEVPEETKMVIKKGLEFKDGMNVLGLIGFFIAFGIAMGKMGDQAKLMVDFFNILNEIVMKLVIMIMWYSPLGIACLICGKIIAIKDLEVVARQLGMYMVTVIIGLIIHGGIFLPLIYFVVTRKNPFSFFAGIFQAWITALGTASSAGTLPVTFRCLEENLGIDKRVTRFVLPVGATINMDGTALYEAVAAIFIAQMNGVVLDGGQIVTVSLTATLASVGAASIPSAGLVTMLLILTAVGLPTEDISLLVAVDWLLDRMRTSVNVVGDSFGAGIVYHLSKSELDTIDSQHRVHEDIEMTKTQSIYDDMKNHRESNSNQCVYAAHNSVIVDECKVTLAANGKSADCSVEEEPWKREK

SEQ ID NO: 27 - DNA, 21 bp, artificial sequence
  siRNA targetting CD44-SLC1A2 fusion site
cgcagaucgugccaacaauuu

SEQ ID NO: 28 - DNA, 21 bp, artificial sequence
  siRNA targetting CD44-SLC1A2 fusion site 2
gcacaucgugccaacaauauu

SEQ ID NO: 29 - DNA, 52 bp, Homo sapiens
  variation (27)..(27): r = a/g
ctccaacaggaaggacgaatctagtcrcacgcttatcaatccccagattttc

SEQ ID NO: 30 - DNA, 52 bp, Homo sapiens
  variation (27)..(27): r = a/g
gaagtaagaacaaggccacttaccacrtgatcatgatcactaacttcattac

SEQ ID NO: 31 - DNA, 52 bp, Homo sapiens
  variation (27)..(27): m = a/c
cgaaaggtgacaggcaaagttccagcmctgagaacaaagagaaagagagaga

SEQ ID NO: 32 - DNA, 52 bp, Homo sapiens
  variation (27)..(27): w = a/t
cgaggaggccaacgcaaccagcgctgwtgtctctctgttgaacgagactgtg

(19) United States
(12) Patent Application Publication
(10) Pub. No.: US 2012/0258998 A1
(43) Pub. Date: Oct. 11, 2012

(54) Fusion genes in gastrointestinal cancer

(76) Inventors: Patrick Tan, Singapore (SG); Jiong Tao, Singapore (SG)

(21) Appl. No.: 13/066,087

(22) Filed: Apr. 5, 2011

(51) Int. Cl. A61K 31/7088; C12N 15/63; C12N 5/071; C12N 1/00; A61P 35/00; C12Q 1/02; G01N 33/566; C07H 21/04; C07K 14/47; C07K 16/18; C12N 5/09; C12Q 1/68

(52) U.S. Cl. 514/44 A; 435/375; 435/320.1; 435/325; 435/243; 435/6.13; 435/29; 435/6.1; 514/44 R; 435/7.92; 536/23.4; 530/350; 530/324; 536/24.5; 530/387.7; 536/23.1

(57) ABSTRACT

A method of treating or preventing a gastrointestinal cancer by inhibiting expression of a CD44-SLC1A2 fusion gene or a functional variant thereof, or by inhibiting the activity of a fusion protein expressed by the CD44-SLC1A2 fusion gene or the functional variant thereof.

### TECHNICAL FIELD

The present invention refers to the area of biochemistry. In particular the biochemistry of cancer, such as gastrointestinal cancer.

### BACKGROUND

Gastrointestinal cancers are a prominent worldwide cause of malignant diseases and mortality. Prognosis of these cancers varies and depends entirely on the specific type of cancer. Compounding to the lack of a specific diagnostic method for early detection of various gastrointestinal cancers is the lack of specific treatments for patients with gastrointestinal cancer. Treatment of gastrointestinal cancers generally includes chemotherapy or radiotherapy. However, chemotherapy or radiotherapy has been known to have adverse side effects that range from minor, such as nausea or vomiting to major, such as increased susceptibility to infections. Furthermore, the current strategies for treating certain kinds of gastrointestinal cancer, such as gastric (stomach) cancer are far from optimal, with conventional surgery and chemotherapy regimes conferring modest survival benefits with median survival times of 7 to 10 months. Accordingly, there is a need to provide improved methods for treating gastrointestinal cancer.

### SUMMARY OF INVENTION

According to a first aspect, there is provided a method of treating or preventing a gastrointestinal cancer by inhibiting expression of a CD44-SLC1A2 fusion gene or a functional variant thereof, or by inhibiting the activity of a fusion protein expressed by the CD44-SLC1A2 fusion gene or the functional variant thereof.

According to a second aspect, there is provided a method of sensitizing cells of a patient suffering from a gastrointestinal cancer to a cancer treatment by inhibiting expression of a SLC1A2 gene, or a CD44-SLC1A2 fusion gene, or a functional variant of these genes; or by inhibiting the activity of a fusion protein expressed by the CD44-SLC1A2 fusion gene or its functional variant, or the protein expressed by the SLC1A2 gene or its functional variant.

According to a third aspect, there is provided a method of reducing intracellular glutamate level of a cell by inhibiting expression of a CD44-SLC1A2 fusion gene or a functional variant thereof, or by inhibiting the activity of a fusion protein expressed by the CD44-SLC1A2 fusion gene or the functional variant thereof.

According to a fourth aspect, there is provided an isolated nucleic acid encoding a CD44-SLC1A2 fusion protein or a functional variant thereof.

According to a fifth aspect, there is provided a vector comprising the isolated nucleic acid of the fourth aspect.

According to a sixth aspect, there is provided a host cell comprising a vector according to the fifth aspect.

According to a seventh aspect, there is provided an isolated fusion protein encoded by CD44-SLC1A2 fusion gene.

According to an eighth aspect, there is provided siRNA directed against the nucleic acid transcribed from the CD44-SLC1A2 fusion gene.

According to a ninth aspect, there is provided an antibody, a functional variant thereof, or a fragment of the antibody capable of binding to the fusion protein according to the seventh aspect.

According to a tenth aspect, there is provided a pharmaceutical composition comprising an inhibitor of CD44-SLC1A2 fusion gene or a functional variant thereof, or an inhibitor for a fusion protein expressed by the CD44-SLC1A2 fusion gene or the functional variant thereof.

According to an eleventh aspect, there is provided a pharmaceutical composition comprising an inhibitor of SLC1A2 gene or a functional variant thereof, or an inhibitor for a protein expressed by the SLC1A2 gene or the functional variant thereof, and a compound for cancer treatment.

According to a twelfth aspect, there is provided method of identifying a compound that modulates expression of CD44-SLC1A2 fusion gene in a cell, the method comprising: (a) exposing cells expressing CD44-SLC1A2 fusion gene with a test compound; (b) determining the expression level of CD44-SLC1A2 fusion gene in the cells which were exposed to the test compound under (a); (c) comparing the level of expression of the CD44-SLC1A2 fusion gene determined under (b) with the expression of the CD44-SLC1A2 fusion gene in control cells which were not exposed to the test compound; wherein a difference in the expression level between the cells under (b) compared to the control cells identifies the compound that modulates expression of CD44-SLC1A2 fusion gene in a cell.

According to a thirteenth aspect, there is provided a method of identifying a compound that modulates the amount of a fusion protein encoded by CD44-SLC1A2 fusion gene comprised in a cell, the method comprising: (a) exposing cells expressing the fusion protein with a test compound; (b) determining the amount of the fusion protein in the cells which were exposed to the test compound under (a); (c) comparing the amount of CD44-SLC1A2 fusion protein determined under (b) with the amount of CD44-SLC1A2 fusion protein in control cells not exposed to the test compound; wherein a difference in the amount of the CD44-SLC1A2 fusion protein between the cells under (b) compared to the control cells identifies the compound that modulates the amount of CD44-SLC1A2 fusion protein in the cells.

According to a fourteenth aspect, there is provided a prognostic method for determining the clinical outcome of a patient with gastrointestinal cancer, wherein the method comprises identifying and determining the level of expression of CD44-SLC1A2 fusion gene, or the presence of a CD44-SLC1A2 fusion gene, or both, or functional variants thereof in the patient, wherein a high CD44-SLC1A2 expression or presence indicates that the person may develop distinct clinical outcomes from tumors with low CD44-SLC1A2 expression.

According to a fifteenth aspect, there is provided a method of assessing the risk of a patient to develop gastrointestinal cancer, wherein the method comprises identifying the presence of a CD44-SLC1A2 fusion protein or the presence of a CD44-SLC1A2 fusion gene or both or functional variants thereof in a patient.

According to a sixteenth aspect, there is provided a kit for use in treating or preventing gastrointestinal cancer in a patient, said kit comprises one of the following selected from the group consisting of an isolated nucleic acid according to the fourth aspect, a vector according to the fifth aspect, a host cell according to the sixth aspect, an isolated fusion protein according to the seventh aspect, an siRNA according to the eight aspect, an antibody according to a ninth aspects, and pharmaceutical compositions according to the tenth and eleventh aspects.

### SEQUENCE LISTING

SEQ ID NO: 1: SLC1A2 exon 3 primer for outer 5′ PCR.

SEQ ID NO: 2: SLC1A2 exon 2 primer for inner 5′ PCR.

SEQ ID NO: 3: forward primer to CD44 exon 1.

SEQ ID NO: 4: reverse primer to SLC1A2 exon 3.

SEQ ID NO: 5; reverse primer to SLC1A2 exon 4.

SEQ ID NO: 6: reverse primer to SLC1A2 exon 5.

SEQ ID NO: 7: reverse primer to SLC1A2 exon 6.

SEQ ID NO: 8: forward primer for detection of CD44/SLC1A2 chromosomal inversion, forward primer at CD44 exon 1, positive strand.

SEQ ID NO: 9; a reverse primer for detection of CD44/SLC1A2 chromosomal inversion, reverse primer at SLC1A2 intron 1 in the minimal breakpoint region, negative strand.

SEQ ID NO: 10: SLC1A2 exon 1 primer for outer 3′ PCR.

SEQ ID NO: 11: SLC1A2 exon 1 primer for inner 3′ PCR.

SEQ ID NO: 12: forward primer for detection of GAPDH.

SEQ ID NO: 13 reverse primer for detection of GAPDH.

SEQ ID NO: 14: forward primer for detection of CD44 exon 2.

SEQ ID NO: 15: reverse primer for detection of CD 44 exon 2.

SEQ ID NO: 16: forward primer for detection of SLC1A2 exon 1.

SEQ ID NO: 17: reverse primer for detection of SLC1A2 exon 1.

SEQ ID NO: 18: forward primer for detection of fusion CD44 exon 1.

SEQ ID NO: 19: reverse primer for detection of fusion SLC1A2 exon 2.

SEQ ID NO: 20: sequence of protein translation initiated from ATG site in the CD44 exon 1 (65 amino acids protein expressed by the CD44-SLC1A2 fusion gene).

SEQ ID NO: 21: sequence of protein translation initiated from ATG site in SLC1A2 exon 2 (565 amino acids truncated SLC1A2 protein (CD44-SLC1A2 fusion protein) that is 17 amino acids shorter than the full length form).

SEQ ID NO: 22: sequence of wild type CD44 gene.

SEQ ID NO: 23: sequence of CD44-SLC1A2 fusion gene.

SEQ ID NO: 24: sequence of wild type SLC1A2 gene.

SEQ ID NO: 25: sequence of wild type CD44 protein.

SEQ ID NO: 26: sequence of wild type SLC1A2 protein.

SEQ ID NOs: 27-28: sequences of siRNAs targeting the CD44-SLC1A2 fusion site.

SEQ ID NOs: 29-32: sequences of single nucleotide polymorphic (SNP) nucleotide of SLC1A2.

### DEFINITIONS

As used herein, the term “prevention” of gastrointestinal cancer refers to the act of preventing or hindering gastrointestinal cancer from occurring. In that case, administering a composition as described herein has the effect that gastrointestinal cancer cannot develop in the patient. Prevention includes methods in which the risk of developing disease or condition is reduced and also includes reduction in the risk of developing a disease or condition and/or a prevention of worsening of symptoms or progression of a disease or reduction in the risk of worsening of symptoms or progression of a disease. Prevention is to be differentiated from “treatment of gastrointestinal cancer” or just “cancer treatment” in which a composition referred to herein is used for the treatment of gastrointestinal cancer cells which already exist in the patient or in other words for the treatment of a patient already suffering from a gastrointestinal cancer. The term “chemoprevention” may also be used. Like the term cancer treatment, “chemoprevention” also refers to the treatment of a patient already suffering from gastrointestinal cancer and is not to be mistaken with the “prevention of gastrointestinal cancer” as referred to in some of the claims of the present invention.

Chemoprevention indicates that a treatment is supposed to avoid the use of chemotherapy which has mostly severe side effects for the animal body undergoing this specific kind of treatment. In another embodiment, the inhibitors and compositions comprising them referred to herein can also be used for preventing recurrence or relapse of gastrointestinal cancer after undergoing a gastrointestinal cancer treatment. That means that an animal body which suffered from gastrointestinal cancer and which underwent a treatment to heal the animal body from gastrointestinal cancer uses the inhibitors referred to herein to prevent gastrointestinal cancer from reoccurring. In one example it means that the animal body underwent and finished a treatment to heal or cure the animal body from gastrointestinal cancer. The difference to an ongoing gastrointestinal cancer treatment is based on the fact that the compositions would not be used to destroy or stop proliferation of cancer cells but, as for the “prevention of gastrointestinal cancer” to prevent or hinder the gastrointestinal cancer from reoccurring. “Cure” or “heal” as referred to herein is defined clinically as the permanent absence of signs or symptoms of cancer; complete remission or complete response as disappearance of clinical evidence of cancer.

In general, “cancer” is considered to refer to a group of cells (usually derived from a single cell) that has lost its normal control mechanisms and thus has unregulated growth (proliferation), lack of differentiation, local tissue invasion, and, often, metastasis. Cancerous (malignant) cells can develop from any tissue within any organ. As cancerous cells grow and multiply, they form a mass of cancerous tissue—called the tumor—that can invade and destroy normal adjacent tissues. The term “tumor” refers to an abnormal growth or mass, tumors can be cancerous or noncancerous. Cancerous cells from the primary (initial) site can spread (metastasize) throughout the body. Cancerous cells develop from healthy cells in a complex process called transformation. The first step in the process is initiation, in which a change in the cells genetic material (in the DNA or sometimes in the chromosome structure) primes the cell to become cancerous. The change in the cell's genetic material may occur spontaneously or be brought on by an agent that causes cancer (a carcinogen).

As used herein the term “effective amount” includes within its meaning a non-toxic but sufficient amount of an agent or compound to provide the desired effect. The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

The term “sensitizing” as used herein refers to a method of increasing susceptibility of a cell, organ or subject to a certain treatment than before. For example, a therapeutic method for treating or preventing cancer as used herein, wherein the chemotherapeutic agent had no effect or only when used at higher doses, could be used or used at lower doses after “sensitizing” the cell, organ or subject using the method as described herein.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. All patents, patent applications, published applications and publications, websites and other published materials referred to throughout the entire disclosure herein, unless noted otherwise, are incorporated by reference in their entirety.

### DETAILED DESCRIPTION

The cancer-specific nature of fusion genes and transcripts have earned them an important place in many translational cancer research applications, including molecular sub-typing, monitoring for disease relapse, and as novel drug targets. In pediatric acute lymphoblastic leukemia (ALL), expression of the AML-ETO and PML-RAR fusion genes are routinely used to diagnose particular clinical subtypes, and treatment of CML (chronic myelogenous leukemia) has been revolutionized by imatinib, an inhibitor of the BCR-ABL fusion gene. It was noted that even low frequency events in cancer can prove therapeutically useful, as shown EML4-ALK fusions in lung cancer (1-5%) and RAF fusions in gastric, melanoma and prostate cancers.

The inventors of the present invention surprisingly found that CD44-SLC1A2 represents another recurrent gene fusion identified in a gastrointestinal cancer, providing evidence for the existence of this important class of molecular aberrations in gastrointestinal malignancies. Furthermore, as a cell membrane-bound receptor, CD44-SLC1A2 may prove amenable to targeting either using small molecules or therapeutic antibodies.

Thus, in one aspect the present invention provides a method of treating or preventing a gastrointestinal cancer by inhibiting expression of a CD44-SLC1A2 fusion gene or a functional variant thereof, or by inhibiting the activity of a fusion protein expressed by the CD44-SLC1A2 fusion gene or the functional variant thereof. The prevention may comprise the prevention of development of gastrointestinal cancer in a patient who was not diagnosed with a gastrointestinal cancer before or preventing relapse (recurrence) of a gastrointestinal cancer in a patient who was previously diagnosed with a gastrointestinal cancer and was treated to cure the gastrointestinal cancer.

Furthermore, some evidence has implicated glutamate and glutamine as a critical amino acid necessary for the maintenance and elaboration of many cancer-specific traits. For example, glutamate and glutamine have been shown to regulate tumor growth and oncogenic signals such as mTOR. The requirement of cancer cells for glutamate may also be related to the Warburg effect—a universal feature of cancer cells where they exhibit overactive glycolysis due to a deficiency in channeling glycolytic metabolites into the TCA cycle for ATP generation. Besides glycolysis, glutamate may provide cancer cells with an alternative route of ATP production since intracellular glutamate and glutamine can also be converted into alpha-ketoglutarate, a TCA cycle intermediate. Glutamate levels have been shown to be elevated in many cancers, and the inventors of the present invention found that glutamate levels are also elevated in gastrointestinal cancer, such as but not limited to gastric tumors. Therefore, inhibiting the expression of CD44-SLC1A2 fusion gene or functional variant thereof may provide be useful in such cancer patients.

Thus, in another aspect, the present invention provides a method of reducing intracellular glutamate level of a cell by inhibiting expression of a CD44-SLC1A2 fusion gene or a functional variant thereof, or by inhibiting the activity of a fusion protein expressed by the CD44-SLC1A2 fusion gene or the functional variant thereof. As discussed above, reducing intracellular glutamate level of a cell may be useful in the treating all kinds of cancers. Exemplary cancer wherein reducing intracellular glutamate level may be useful includes, but is not limited to, gastrointestinal cancers.

Furthermore, the inventors of the present invention also found that targeting CD44-SLC1A2 fusion gene or fusion protein and wild-type SLC1A2 in fusion positive tumors increases the sensitivity of gastrointestinal cancers to commonly used standard-of-care therapies. Accordingly, in another aspect, the present invention provides a method of sensitizing cells of a patient suffering from a gastrointestinal cancer to a cancer treatment by inhibiting expression of a SLC1A2 gene, or a CD44-SLC1A2 fusion gene, or a functional variant of these genes; or by inhibiting the activity of a fusion protein expressed by the CD44-SLC1A2 fusion gene or its functional variant, or the protein expressed by the SLC1A2 gene or its functional variant. In one embodiment, the method of sensitizing cells comprises the step of (1) knockdown of the expression of CD44-SLC1A2 fusion gene; and (2) treating the patient with a cancer treatment. The inhibition or knockdown of SLC1A2 gene, or CD44-SLC1A2 fusion gene may be performed before or at the same time as the cancer treatment. The cancer treatment of the present invention may be a chemotherapeutic agent such as cisplatin. Other exemplary chemotherapeutic agents that may be used in the present disclosure are discussed further below.

In general, gastrointestinal cancer as described herein may be a metastatic or non-metastatic cancer that includes, but is not limited to, cancer of gastrointestinal tract, liver cancer, cancer of biliary tract, pancreas cancer and cancer of the peritoneum. The cancer of the gastrointestinal tract may include, but is not limited to, cancer of the upper gastrointestinal tract, such as squamous cell carcinoma or adenocarcinoma in the esophagus, gastric carcinoma, signet ring cell carcinoma, gastric lymphoma (MALT lymphoma), linitis plastica in the stomach, cancer of the lower gastrointestinal tract such as cancer in small intestine: duodenal cancer, cancer in the appendix: carcinoid, pseudomyxoma peritonei; cancer in the colon/rectum: colorectal polyp such as Peutz-Jeghers syndrome, juvenile polyposis syndrome, familial adenomatous polyposis/gardner's syndrome, Cronkhite-Canada syndrome, colorectal adenocarcinoma, familial adenomatous polyposis, hereditary nonpolyposis colorectal cancer; cancer in the anus: squamous cell carcinoma, gastrointestinal stromal tumor and Krukenberg tumor. Liver cancer may include, but is not limited to, hepatocellular carcinoma (Fibrolamellar), hepatoblastoma, hepatocellular adenoma, cavernous hemangioma, focal nodular hyperplasia, nodular regenerative hyperplasia. Biliary tract cancer may include, but is not limited to, cholangiocarcinoma, klatskin tumor and gallbladder cancer. Pancreatic cancer may include, but is not limited to, cancer in exocrine pancreas, such as: adenocarcinoma, pancreatic ductal carcinoma, cystic neoplasms such as: Serous microcystic adenoma, intraductal papillary mucinous neoplasm, mucinous cystic neoplasm, solid pseudopapillary neoplasm and pancreatoblastoma. Cancer in the peritoneum may include, but is not limited to primary peritoneal carcinoma, peritoneal mesothelioma and desmoplastic small round cell tumor.

The present invention also refers to different inhibitors or combination compositions comprising one or more inhibitors and one or more additional pharmaceutical active compositions. Inhibitors can include, but are not limited to RNA silencing agents, such as interfering ribonucleic acids, immunoglobulin's and anticalins and small organic molecules.

Reference to inhibitors can refer to any of these molecules unless specified otherwise or derivable from the specific content of the specification.

In one aspect the present invention also relates to nucleic acid molecules that encode a CD44-SLC1A2 fusion protein or antibody used as inhibitor for a CD44-SLC1A2 fusion protein. Nucleic acid molecules of the invention irrespective of whether those nucleic acid molecules refer to the target genes itself or the nucleic acid based inhibitors, such a interfering ribonucleic acid, are not limited to the sequences disclosed herein, but also include functional variants thereof.

For example, functional nucleic acid variants of the target genes CD44-SLC1A2 and wild-type SLC1A2 may include natural or synthetic polymorphism. The term “polymorphism” as used herein refers to the occurrence of two or more genetically determined alternative sequences or alleles in a population. A polymorphism may comprise one or more nucleic acid base changes, an insertion, a repeat, or a deletion. A polymorphic locus may be as small as one nucleic acid base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. Single nucleotide polymorphisms (SNPs) are included in polymorphisms. Exemplary polymorphisms in SLC1A2 gene include, but are not limited to synonymous and nonsynonymous SNPs such as SEQ ID NOs: 29-32. Accordingly, those polymorphisms can also be found in the CD44-SLC1A2 fusion gene.

In addition, functional nucleic acid variants within the invention may be defined by reference to their physical properties in hybridization. The skilled worker will recognize that nucleic acids can be used to identify its complement and, since some nucleic acid, such as DNA is double stranded, its equivalent or homolog, using nucleic acid hybridization techniques. It also will be recognized that hybridization can occur with less than 100% complementarity. However, given appropriate choice of conditions, hybridization techniques can be used to differentiate among nucleic acid sequences based on their structural relatedness to a particular probe.

Structural similarity between two nucleic acid sequences can be expressed as a function of “stringency” of the conditions under which the two sequences will hybridize with one another. As used herein, the term “stringency” refers to the extent that the conditions disfavor hybridization. Stringent conditions strongly disfavor hybridization, and only the most structurally related molecules will hybridize to one another under such conditions. Conversely, non-stringent conditions favor hybridization of molecules displaying a lesser degree of structural relatedness. Hybridization stringency, therefore, directly correlates with the structural relationships of two nucleic acid sequences. The following relationships are useful in correlating hybridization and relatedness (where Tₘ is the melting temperature of a nucleic acid duplex):

a. Tₘ=69.3+0.41(G+C) %

b. The Tₘ, of a duplex nucleic acid decreases by 1° C. with every increase of 1% in the number of mismatched base pairs.

c. (Tₘ)μ2−(Tₘ)μ,=18.5 log₁₀μ2/μl where μ1 and μ2 are the ionic strengths of two solutions.

Hybridization stringency is a function of many factors, including overall nucleic acid concentration, ionic strength, temperature, probe size and the presence of agents which disrupt hydrogen bonding. Factors promoting hybridization include high nucleic acid concentrations, high ionic strengths, low temperatures, longer probe size and the absence of agents that disrupt hydrogen bonding. Hybridization typically is performed in two phases: the “binding” phase and the “washing” phase.

First, in the binding phase, the probe is bound to the target under conditions favoring hybridization. Stringency is usually controlled at this stage by altering the temperature. For high stringency, the temperature is usually between 65° C. and 70° C., unless short (<20 nt) oligonucleotide probes are used. A representative hybridization solution comprises 6×SSC, 0.5% SDS, 5×Denhardt's solution and 100 μg of nonspecific carrier nucleic acid. Of course, many different, yet functionally equivalent, buffer conditions are known. Where the degree of relatedness is lower, a lower temperature may be chosen. Low stringency binding temperatures are between about 25° C. and 40° C. Medium stringency is between at least about 40° C. to less than about 65° C. High stringency is at least about 65° C.

Second, the excess probe is removed by washing. It is at this phase that more stringent conditions usually are applied. Hence, it is this “washing” stage that is most important in determining relatedness via hybridization of nucleic acid strands. Washing solutions typically contain lower salt concentrations. One exemplary medium stringency solution contains 2×SSC and 0.1% SDS. A high stringency wash solution contains the equivalent (in ionic strength) of less than about 0.2×SSC, with a preferred stringent solution containing about 0.1×SSC. The temperatures associated with various stringencies are the same as discussed above for “binding.” The washing solution also typically is replaced a number of times during washing. For example, typical high stringency washing conditions comprise washing twice for 30 minutes at 55° C. and three times for 15 minutes at 60° C.

Accordingly, the present invention includes nucleic acid molecules encoding CD44-SLC1A2 fusion protein, SLC1A2 protein or encoding antibodies capable of binding CD44-SLC1A2 fusion protein that hybridize under high stringency binding and washing conditions. Exemplary molecules (from an mRNA perspective) are those that have at least 90%, 91%, 92%, 93%, 94%, 95%, 96%, 97%, 98% or 99% sequence identity with one of the nucleic acid molecules described herein.

Thus, in one aspect, the present invention provides nucleic acid encoding a CD44-SLC1A2 fusion protein, SLC1A2 or a functional variant thereof. Exemplary nucleic acid encoding a CD44-SLC1A2 fusion protein may include the nucleic acid sequence SEQ ID NO: 23.

The present invention further provides recombinant nucleic acid or DNA constructs comprising one or more of the nucleotide sequences referred to in the present invention. The recombinant constructs of the present invention can be used in connection with a vector, such as a plasmid, phagemid, phage or viral vector, into which a nucleic acid molecule encoding at least one of an inhibitory antibody or a CD44-SLC1A2 fusion protein or SLC1A2 protein is inserted.

The present invention further provides host cells containing at least one of the nucleic acids referred to in the present invention. The host cell can be virtually any cell for which expression vectors are available. It may be, for example, a higher eukaryotic host cell, such as a mammalian cell, a lower eukaryotic host cell, such as a yeast cell or a prokaryotic cell, such as a bacterial cell.

In another aspect, there is provided an isolated fusion protein encoded by CD44-SLC1A2 fusion gene, or a functional variant thereof of the protein. The isolated fusion protein may include amino acid sequence of SEQ ID NO: 21.

With a functional variant of the CD44-SLC1A2 fusion protein or SLC1A2 it is meant a protein or polypeptide which refers to an amino acid polymer, comprising natural and/or non-natural amino acids, including L- and D-isomeric forms as are well understood in the art. For example, CD44-SLC1A2 fusion protein or SLC1A2 wild-type protein may be referred to as either a protein or polypeptide.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native, chemical synthetic or recombinant form. A “fragment” is a segment, domain, portion or region of the CD44-SLC1A2 fusion protein, which constitutes less than 100% of the full-length protein.

Functional protein or polypeptide variants may be made that conserve the overall molecular structure of an isolated CD44-SLC1A2 fusion protein described herein. Given the properties of the individual amino acids, some rational amino acid substitutions will be recognized by the skilled worker for a functional variant of the CD44-SLC1A2 fusion protein. Amino acid substitutions, i.e., “conservative substitutions,” may be made, for instance, on the basis of similarity in polarity, charge, solubility, hydrophobicity, hydrophilicity, and/or the amphipathic nature of the residues involved.

For example, (a) nonpolar (hydrophobic) amino acids include alanine, leucine, iso-leucine, valine, proline, phenylalanine, tryptophan, and methionine; (b) polar neutral amino acids include glycine, serine, threonine, cysteine, tyrosine, asparagine, and glutamine; (c) positively charged (basic) amino acids include arginine, lysine, and histidine; and (d) negatively charged (acidic) amino acids include aspartic acid and glutamic acid. Substitutions typically may be made within groups (a)-(d). In addition, glycine and proline may be substituted for one another based on their ability to disrupt α-helices. Similarly, certain amino acids, such as alanine, cysteine, leucine, methionine, glutamic acid, glutamine, histidine and lysine are more commonly found in α-helices, while valine, isoleucine, phenylalanine, tyrosine, tryptophan and threonine are more commonly found in β-pleated sheets. Glycine, serine, aspartic acid, asparagine, and proline are commonly found in turns. Some preferred substitutions may be made among the following groups: (i) S and T; (ii) P and G; and (iii) A, V, L and I. Given the known genetic code, and recombinant and synthetic DNA techniques, the skilled scientist readily can construct DNAs encoding the conservative amino acid variants.

Functional variants of CD44-SLC1A2 fusion protein comprise a high sequence identity to ensure that they maintain the biological activity. As used herein, “sequence identity” between two polypeptide sequences, indicates the percentage of amino acids that are identical between the sequences. “Sequence homology”, indicates the percentage of amino acids that either are identical or that represent conservative amino acid substitutions. Preferred polypeptide sequences of the invention have a sequence identity of at least 90%, or 91%, or 92%, or 93%, or 94%, or 95%, or 96%, or 97%, or 98%, or 99%.

CD44-SLC1A2 fusion proteins or functional variants thereof can be produced by any method known to those of skill in the art including in vivo and in vitro methods. Desired proteins can be expressed in any organism suitable to produce the required amounts and forms of the proteins, such as for example, needed for administration and treatment. Expression hosts include prokaryotic and eukaryotic organisms such as E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification. A discussion on exemplary host cell is provided below.

Many expression vectors are available and known to those of skill in the art and can be used for expression of proteins. The choice of expression vector will be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector. Exemplary vectors are discussed below.

A variety of host-vector systems can be used to express the protein coding sequence. These include but are not limited to mammalian cell systems infected with virus (e.g. vaccinia virus, adenovirus and other viruses); insect cell systems infected with virus (e.g. baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system used, any one of a number of suitable transcription and translation elements can be used.

Any methods known to those of skill in the art for the insertion of DNA fragments into a vector can be used to construct expression vectors containing a chimeric gene containing appropriate transcriptional/translational control signals and protein coding sequences. These methods can include in vitro recombinant DNA and synthetic techniques and in vivo recombinants (genetic recombination).

Expression of nucleic acid sequences encoding protein or functional variants thereof can be regulated by a second nucleic acid sequence so that the nucleic acids are expressed in a host transformed with the recombinant DNA molecule(s). For example, expression of the proteins can be controlled by any promoter/enhancer known in the art.

The present invention also provides host cells comprising the expression vectors as described herein. Host cells in the present invention may include prokaryotic or eukaryotic cell, strain, species or genera suitable for introduction and for expression of transgenic nucleic acid sequences. Exemplary host cells may include mammalian cells, such as human, monkey or mice. Cell lines that may be used in the present invention include, but are not limited to gastric cancer cell lines AGS, KATO III, SNU1, SNU16, N87, HFE145, HS746T, AZ521, Ist1, TMK1, MKN1, MKN7, MKN28, MKN45, MKN74, Fu97, SCH, YCC and IM95.

Useful expression vectors for bacterial use are constructed by inserting a structural DNA sequence encoding a desired protein together with suitable translation initiation and termination signals in operable reading phase with a functional promoter. The vector can comprise one or more phenotypic selectable markers and an origin of replication to ensure maintenance of the vector and, if desirable, to provide amplification within the host. Suitable prokaryotic hosts for transformation include E. coli, Bacillus subtilis, Salmonella typhimurium and various species within the genera Pseudomonas, Streptomyces, and Staphylococcus.

Bacterial vectors may be, for example, bacteriophage-, plasmid- or phagemid-based. These vectors can contain a selectable marker and bacterial origin of replication derived from commercially available plasmids typically containing elements of the well known cloning vectors. Following transformation of a suitable host strain and growth of the host strain to an appropriate cell density, the selected promoter is de-repressed/induced by appropriate means (e.g., temperature shift or chemical induction) and cells are cultured for an additional period. Cells are typically harvested by centrifugation, disrupted by physical or chemical means, and the resulting crude extract retained for further purification. In bacterial systems, a number of expression vectors may be advantageously selected depending upon the use intended for the protein being expressed. For example, when a large quantity of such a protein is to be produced, for the generation of the fusion protein or functional variants to screen peptide libraries, for example, vectors which direct the expression of high levels of such protein products that are readily purified may be desirable.

Accordingly, in one aspect, the nucleic acid of the present invention may be expressed using an expression vector. Vectors as disclosed in the present invention may comprise a nucleic acid of the present invention. The above comments with respect to the CD44-SLC1A2 fusion protein, and SLC1A2 wild-type protein, and modifications to obtain functional variants thereof also apply to the antibodies of the present invention.

An example of an agent capable of downregulating the polypeptides of the present invention is an RNA silencing agent.

As used herein, the term “RNA silencing” refers to a group of regulatory mechanisms (e.g. RNA interference (iRNA) (or knockdown), transcriptional gene silencing (TGS), post-transcriptional gene silencing (PTGS), quelling, co-suppression, and translational repression) mediated by RNA silencing agents which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of inhibiting or “silencing” the expression of a target gene, such as the CD44-SLC1A2 fusion gene or the SLC1A2. In certain embodiments, the RNA silencing agent is capable of preventing complete processing (e.g., the full translation and/or expression) of an mRNA encoding the target gene through a post-transcriptional silencing mechanism. RNA silencing agents include interfering ribonucleic acids. Exemplary interfering ribonucleic acids include dsRNAs, such as siRNAs, miRNAs and shRNAs. In one embodiment, the RNA silencing agent is capable of inducing RNA interference. In another embodiment, the RNA silencing agent is capable of mediating translational repression.

An RNA silencing agent can be rationally designed based on sequence information and desired characteristics. For example, an RNA silencing agent can be designed according to the relative melting temperature of the candidate duplex. Generally, the duplex should have a lower melting temperature at the 5′ end of the antisense strand than at the 3′ end of the antisense strand. Candidate RNA silencing agents can also be designed by performing, for example, a gene walk analysis of the genes that will serve as the target gene. Overlapping, adjacent, or closely spaced candidate agents corresponding to all or some of the transcribed region can be generated and tested. Each of the RNA silencing agents can be tested and evaluated for the ability to down regulate the target gene expression.

Accordingly, the present invention also contemplates use of dsRNA, such as siRNA to downregulate protein expression from mRNA. The dsRNA may be from 15 to 35 base pairs, from 15 to 25 base pairs, from about 15 to 25 bp, from about 15 to 21 bp. The sequence may comprise or consist of 15, 16, 17, 18, 19, 20, 21, 22, 23, 24, 25, 26, 27, 28, 29 or 30 base pairs. The dsRNA may be a siRNA that targets CD44-SLC1A2 fusion gene. In one embodiment siRNA may be encoded by the sequences SEQ ID NOs: 27-28.

A RNA silencing agent described herein includes more than one, and preferably two, strands in which interstrand hybridization can form a region of duplex structure. A “strand” herein refers to a contiguous sequence of nucleotides (including non-naturally occurring or modified nucleotides). The two or more strands may be, or each form a part of, separate molecules, or they may be covalently interconnected, e.g. by a linker, e.g. a polyethylene glycol linker, to form but one molecule. At least one strand can include a region which is sufficiently complementary to a target RNA, such as the mRNA transcribed from the CD44-SLC1A2 fusion gene or the SLC1A2 gene. Such strand is termed the “antisense strand”. A second strand comprised in the dsRNA agent which comprises a region complementary to the antisense strand is termed the “sense strand”. However, a RNA silencing agent can also be formed from a single RNA molecule which is, at least partly; self-complementary, forming, e.g., a hairpin or panhandle structure, including a duplex region. In such case, the term “strand” refers to one of the regions of the RNA molecule that is complementary to another region of the same RNA molecule.

Accordingly, in one embodiment, inhibition of any of the genes referred in the present invention may include administering at least one RNA silencing agent as described above. In another embodiment, the RNA silencing agent is an interfering ribonucleic acid. The interfering ribonucleic acid can be siRNA or shRNA or miRNA. In one embodiment the interfering ribonucleic acid is siRNA directed against the mRNA transcribed from the CD44-SLC1A2 fusion gene. An example of such an siRNA can include, but is not limited to a nucleic acid sequence of SEQ ID NOs: 27-28.

RNA silencing agents discussed herein include otherwise unmodified RNA as well as RNA which have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, preferably as occur naturally in the human body. The art has referred to rare or unusual, but naturally occurring, RNAs as modified RNAs. Rare or unusual RNAs, often termed modified RNAs (apparently because they are typically the result of a post-transcriptional modification) are within the term unmodified RNA, as used herein.

Modified RNA as used herein refers to a molecule in which one or more of the components of the nucleic acid, namely sugars, bases, and phosphate moieties, are different from that which occurs in nature, preferably different from that which occurs in the human body. While they are referred to as modified “RNAs,” they will of course, because of the modification, include molecules which are not RNAs. Nucleoside surrogates are molecules in which the ribophosphate backbone is replaced with a non-ribophosphate construct that allows the bases to the presented in the correct spatial relationship such that hybridization is substantially similar to what is seen with a ribophosphate backbone, e.g., non-charged mimics of the ribophosphate backbone. Examples of all of the above are discussed herein.

Modifications described herein can be incorporated into any double-stranded RNA and RNA-like molecule described herein, e.g., an RNA silencing agent. It may be desirable to modify one or both of the antisense and sense strands of an RNA silencing agent. As nucleic acids are polymers of subunits or monomers, many of the modifications described below occur at a position which is repeated within a nucleic acid, e.g., a modification of a base, or a phosphate moiety, or the non-linking O of a phosphate moiety. In some cases the modification will occur at all of the subject positions in the nucleic acid but in many, and in fact in most, cases it will not. By way of example, a modification may only occur at a 3′ or 5′ terminal position, may only occur in a terminal region, e.g. at a position on a terminal nucleotide or in the last 2, 3, 4, 5, or 10 nucleotides of a strand. A modification may occur in a double strand region, a single strand region, or in both. For example a phosphorothioate modification at a non-linking O position may only occur at one or both termini, may only occur in a terminal regions, e.g., at a position on a terminal nucleotide or in the last 2, 3, 4, 5, or 10 nucleotides of a strand, or may occur in double strand and single strand regions, particularly at termini. Similarly, a modification may occur on the sense strand, antisense strand, or both. In some cases, the sense and antisense strand will have the same modifications or the same class of modifications, but in other cases the sense and antisense strand will have different modifications, e.g., in some cases it may be desirable to modify only one strand, e.g. the sense strand.

Two prime objectives for the introduction of modifications into RNA silencing agents is their stabilization towards degradation in biological environments and the improvement of pharmacological properties, e.g., pharmacodynamic properties.

A RNA silencing agent can include an internucleotide linkage (e.g., the chiral phosphorothioate linkage) useful for increasing nuclease resistance. In addition, or in the alternative, a RNA silencing agent can include a ribose mimic for increased nuclease resistance. A RNA silencing agent can also include ligand-conjugated monomer subunits and monomers for oligonucleotide synthesis. A RNA silencing agent can be complexed with an amphipathic moiety.

A RNA silencing agent that targets the mRNA of CD44-SLC1A2 fusion gene or SLC1A2 can have enhanced resistance to nucleases. One way to increase resistance is to identify cleavage sites and modify such sites to inhibit cleavage. For example, the di-nucleotides 5′-ua-3′, 5′-ca-3′, 5′-ug-3′, 5′-uu-3′, or 5′-cc-3′ can serve as cleavage sites. In certain embodiments, all the pyrimidines of a RNA silencing agent carry a 2′-modification in either the sense strand, the antisense strand, or both strands, and the RNA silencing agent therefore has enhanced resistance to endonucleases. Enhanced nuclease resistance can also be achieved by modifying the 5′ nucleotide, resulting, for example, in at least one 5′-uridine-adenine-3′ (5′-ua-3′) di-nucleotide wherein the uridine is a 2′-modified nucleotide; at least one 5′-cytidine-adenine-3′ (5′-ca-3′) di-nucleotide, wherein the 5′-cytidine is a 2′-modified nucleotide; at least one 5′-uridine-guanine-3′ (5′-ug-3′) di-nucleotide, wherein the 5′-uridine is a 2′-modified nucleotide; at least one 5′-uridine-uridine-3′ (5′-uu-3′) di-nucleotide, wherein the 5′-uridine is a 2′-modified nucleotide; or at least one 5′-cytidine-cytidine-3′ (5′-cc-3′) di-nucleotide, wherein the 5′-cytidine is a 2′-modified nucleotide.

The RNA silencing agent can include at least 2, at least 3, at least 4 or at least 5 of such di-nucleotides. In one embodiment, the 5′ nucleotide in all occurrences of the sequence motifs 5′-ua-3′ and 5′-ca-3′ in either the sense strand, the antisense strand, or both strands is a modified nucleotide. In another embodiment the 5′ nucleotide in all occurrences of the sequence motifs 5′-ua-3′, 5′-ca-3′ and 5′-ug-3′ in either the sense strand, the antisense strand, or both strands is a modified nucleotide. In another example, all pyrimidine nucleotides in the sense strand are modified nucleotides, and the 5′ nucleotide in all occurrences of the sequence motifs 5′-ua-3′ and 5′-ca-3′ in the antisense strand are modified nucleotides, or where the antisense strand does comprise neither of a 5′-ua-3′ and a 5′-ca-3′ motif, in all occurrences of the sequence motif 5′-ug-3′.

For increased nuclease resistance and/or binding affinity to the target, an RNA silencing agent, e.g., the sense and/or antisense strands of the RNA silencing agent, can include, for example, 2′-modified ribose units and/or phosphorothioate linkages. For example, the 2′ hydroxyl group (OH) can be modified or replaced with a number of different “oxy” or “deoxy” substituents.

Examples of “oxy”-2′ hydroxyl group modifications include alkoxy or aryloxy (OR, e.g., R—H, alkyl, cycloalkyl, aryl, aralkyl, heteroaryl or sugar); polyethylene glycols (PEG), O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; O-AMINE and aminoalkoxy, O(CH₂)ₙAMINE, (e.g., AMINE=NH₂; alkylamino, dialkyl-amino, heterocyclyl amino, arylamino, diaryl amino, heteroaryl amino, or diheteroaryl amino, ethylene diamine, polyamino). It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

“Deoxy” modifications include hydrogen (i.e. deoxyribose sugars, which are of particular relevance to the overhang portions of partially ds RNA); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl amino, arylamino, diaryl amino, heteroaryl amino, or diheteroaryl amino), —NHC(O)R (R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino functionality. Exemplary substituents include, but are not limited to 2′-methoxyethyl, 2′-OCH₃, 2′-O-allyl, 2′-C-allyl, and 2′-fluoro.

To maximize nuclease resistance, the 2′ modifications can be used in combination with one or more phosphate linker modifications (e.g., phosphorothioate). The so-called “chimeric” oligonucleotides are those that contain two or more different modifications.

The inclusion of furanose sugars in the oligonucleotide backbone can also decrease endonucleolytic cleavage. An RNA silencing agent can be further modified by including a 3′ cationic group, or by inverting the nucleoside at the 3′-terminus with a 3′-3′ linkage. In another alternative, the 3′-terminus can be blocked with an aminoalkyl group, e.g., a 3′ C₅-aminoalkyl dT. Other 3′ conjugates can inhibit 3′-5′ exonucleolytic cleavage. While not being bound by theory, a 3′ conjugate, such as naproxen or ibuprofen, may inhibit exonucleolytic cleavage by sterically blocking the exonuclease from binding to the 3′-end of oligonucleotide. Even small alkyl chains, aryl groups, or heterocyclic conjugates or modified sugars (D-ribose, deoxyribose, glucose etc.) can block 3′-5′-exonucleases.

An mRNA agent can have increased resistance to nucleases when a duplexed RNA silencing agent includes a single-stranded nucleotide overhang on at least one end. In one example, the nucleotide overhang includes 1 to 4, preferably 2 to 3, unpaired nucleotides. In one embodiment, the unpaired nucleotide of the single-stranded overhang that is directly adjacent to the terminal nucleotide pair contains a purine base, and the terminal nucleotide pair is a G-C pair, or at least two of the last four complementary nucleotide pairs are G-C pairs.

In further embodiments, the nucleotide overhang may have 1 or 2 unpaired nucleotides, and in one example the nucleotide overhang is 5′-GC-3′. In preferred embodiments, the nucleotide overhang is on the 3′-end of the antisense strand. In another embodiment, the RNA silencing agent includes the motif 5′-CGC-3′ on the 3′-end of the antisense strand, such that a 2-nt overhang 5′-GC-3′ is formed.

Thus, an RNA silencing agent can include monomers which have been modified so as to inhibit degradation, e.g., by nucleases, e.g., endonucleases or exonucleases, found in the body of a subject.

These monomers are referred to herein as NRMs, or Nuclease Resistance promoting Monomers, the corresponding modifications as NRM modifications. In many cases these modifications will modulate other properties of the RNA silencing agent as well, e.g., the ability to interact with a protein, e.g., a transport protein, e.g., serum albumin, or a member of the RISC, or the ability of the first and second sequences to form a duplex with one another or to form a duplex with another sequence, e.g., a target molecule.

Modifications that can be useful for producing RNA silencing agent that meet the preferred nuclease resistance criteria delineated above can include one or more of the following chemical and/or stereochemical modifications of the sugar, base, and/or phosphate backbone:

(i) chiral (SP) thioates. Thus, other NRMs include nucleotide dimers enriched or pure for a particular chiral form of a modified phosphate group containing a heteroatom at the nonbridging position, for example, Sp or Rp, at the position X, where this is the position normally occupied by the oxygen. The atom at X can also be S, Se, or Br₃. When X is S, enriched or chirally pure Sp linkage is preferred. Enriched means at least 70, 80, 90, 95, or 99% of the preferred form;

(ii) attachment of one or more cationic groups to the sugar, base, and/or the phosphorus atom of a phosphate or modified phosphate backbone moiety. Thus, other NRMs include monomers at the terminal position derivatized at a cationic group. As the 5′-end of an antisense sequence should have a terminal —OH or phosphate group this NRM is preferably not used at the 5′-end of an antisense sequence. The group should be attached at a position on the base which minimizes interference with H-bond formation and hybridization, e.g., away from the face which interacts with the complementary base on the other strand;

(iii) nonphosphate linkages at the termini. Thus, other NRMs include non-phosphate linkages, e.g., a linkage of 4 atoms which confers greater resistance to cleavage than does a phosphate bond. Examples include 3′ CH₂—NCH₃—O—CH₂-5′ and 3′ CH₂—NH—(O—)—CH₂-5′;

(iv) 3′-bridging thiophosphates and 5′-bridging thiophosphates. Thus, other NRM's can include these structures;

(v) L-RNA, 2′-5′ linkages, inverted linkages, a-nucleosides. Thus, other NRM's include: L nucleosides and dimeric nucleotides derived from L-nucleosides; 2′-5′ phosphate, non-phosphate and modified phosphate linkages (e.g., thiophosphates, phosphoramidates and boronophosphates); dimers having inverted linkages, e.g., 3′-3′ or 5′-5′ linkages; monomers having an alpha linkage at the 1′ site on the sugar;

(vi) conjugate groups. Thus, some NRM's can include, e.g., a targeting moiety or a conjugated ligand conjugated with the monomer, e.g., through the sugar, base, or backbone;

(vi) abasic linkages. Thus, some NRM's can include an abasic monomer, e.g., an abasic monomer (e.g., a nucleobaseless monomer); an aromatic or heterocyclic or polyheterocyclic aromatic monomer; and

(vii) 5′-phosphonates and 5′-phosphate prodrugs. Thus, some NRM's include monomers, preferably at the terminal position, e.g., the 5′ position, in which one or more atoms of the phosphate group is derivatized with a protecting group, which protecting group or groups, are removed as a result of the action of a component in the subject's body, e.g, a carboxyesterase or an enzyme present in the subject's body. For example a phosphate prodrug in which a carboxy esterase cleaves the protected molecule resulting in the production of a thioate anion which attacks a carbon adjacent to the O of a phosphate and resulting in the production of an unprotected phosphate.

One or more different NRM modifications can be introduced into an RNA silencing agent or into a sequence of a RNA silencing agent. An NRM modification can be used more than once in a sequence or in an RNA silencing agent.

NRM modifications can include some which can be placed only at the terminus and others which can go at any position. Some NRM modifications can inhibit hybridization so it is preferable to use them only in terminal regions, and preferable to not use them at the cleavage site or in the cleavage region of a sequence which targets a target sequence or gene, particularly on the antisense strand.

NRM modifications can be used anywhere in a sense strand, provided that sufficient hybridization between the two strands of the ds RNA silencing agent is maintained. In some embodiments it is desirable to put the NRM at the cleavage site or in the cleavage region of a sense strand, as it can minimize off-target silencing.

In most cases, NRM modifications will be distributed differently depending on whether they are comprised on a sense or antisense strand. If on an antisense strand, modifications which interfere with or inhibit endonuclease cleavage should not be inserted in the region which is subject to RISC mediated cleavage, e.g., the cleavage site or the cleavage region.

Cleavage of the target occurs about in the middle of a 20 or 21 nt antisense strand, or about 10 or 11 nucleotides upstream of the first nucleotide on the target mRNA which is complementary to the antisense strand. As used herein cleavage site refers to the nucleotides on either side of the cleavage site, on the target or on the RNA silencing agent strand which hybridizes to it. Cleavage region means the nucleotides within 1, 2, or 3 nucleotides of the cleavage site, in either direction. Such modifications can be introduced into the terminal regions, e.g., at the terminal position or with 2, 3, 4, or 5 positions of the terminus, of a sense or antisense strand.

Another example of an agent capable of inhibiting the CD44-SLC1A2 fusion protein is an antibody, antibody fragment or anticalin capable of specifically binding the CD44-SLC1A2 fusion protein or the SLC1A2 protein. In one example, the antibody specifically binds at least one epitope of the CD44-SLC1A2 fusion protein or the SLC1A2 protein.

Antibodies (immunoglobulin) or anticalins bind to specific epitopes on the targeted protein. As used herein, the term “epitope” refers to any antigenic determinant on an antigen to which the paratope of an antibody or anticalins binds. Epitopic determinants usually consist of chemically active surface groupings of molecules such as amino acids or carbohydrate side chains and usually have specific three-dimensional structural characteristics, as well as specific charge characteristics. Exemplary epitopes can include amino acids surrounding Val510 and Val501 of human wild-type protein transcribed by SLC1A2.

Antibodies and anticalins capable of binding to CD44-SLC1A2 fusion protein or SLC1A2 can be modified using methods known in the art. For example antibodies described herein can be humanized. Methods for humanizing non-human antibodies are well known in the art. A “humanized antibody” or functional humanized antibody fragment is defined herein as one that is (i) derived from a non-human source (e.g., a transgenic mouse which bears a heterologous immune system), which antibody is based on a human germline sequence; or (ii) chimeric, wherein the variable domain is derived from a non-human origin and the constant domain is derived from a human origin or (iii) CDR-grafted, wherein the CDRs of the variable domain are from a non-human origin, while one or more frameworks of the variable domain are of human origin and the constant domain (if any) is of human origin.

As used herein, an antibody or anticalin “binds specifically to,” is “specific to/for” or “specifically recognizes” an antigen (here, CD44-SLC1A2, SLC1A2 or fragment thereof) if such antibody or anticalin is able to discriminate between such antigen and one or more reference antigen(s), since binding specificity is not an absolute, but a relative property. In its most general form (and when no defined reference is mentioned), “specific binding” is referring to the ability of the antibody to dis-criminate between the antigen of interest and an unrelated antigen, as determined, for example, in accordance with one of the following methods. Such methods comprise, but are not limited to Western blots, ELISA-, RIA-, ECL-, IRMA-tests and peptide scans. For example, a standard ELISA assay can be carried out.

However, “specific binding” also may refer to the ability of an antibody or anticalins to discriminate between the target antigen and one or more closely related antigen(s), which are used as reference points, e.g. between CD44, SLC1A2 and CD44-SLC1A2 fusion protein. Additionally, “specific binding” may relate to the ability of an antibody to discriminate between different parts of its target antigen, e.g. different domains or regions of CD44-SLC1A2 fusion protein, or between one or more key amino acid residues or stretches of amino acid residues of CD44-SLC1A2 fusion protein.

Also, as used herein, an antibody also referred to as immunoglobulin (Ig) can be defined as a protein belonging to the class IgG, IgM, IgE, IgA, or IgD (or any subclass thereof). These classes include all conventionally known antibodies and functional fragments thereof. A “functional fragment” of an antibody/immunoglobulin is defined as a fragment of an antibody/immunoglobulin (e.g., a variable region of an IgG) that retains the antigen-binding region. An “antigen-binding region” of an antibody typically is found in one or more hypervariable region(s) of an antibody, i.e., the CDR-I, -2, and/or -3 regions; however, the variable “framework” regions can also play an important role in antigen binding, such as by providing a scaffold for the CDRs.

“Functional fragments” of antibodies include the domain of a F(ab′)2 fragment, a Fab fragment, scFv or constructs comprising single immunoglobulin variable domains or single domain antibody polypeptides, e.g. single heavy chain variable domains or single light chain variable domains. The F(ab′)2 or Fab may be engineered to minimize or completely remove the intermolecular disulphide interactions that occur between the CHI and CL domains.

An antibody of the invention may be derived from a recombinant antibody library that is based on amino acid sequences that have been designed in silico and encoded by nucleic acids that are synthetically created. In silico design of an antibody sequence is achieved, for example, by analyzing a database of human sequences and devising a polypeptide sequence utilizing the data obtained therefrom.

Antibodies described herein are not limited to the specific peptide sequences provided herein. Rather, the invention also embodies variants of these antibodies. With reference to the instant disclosure and conventionally available technologies and references, the skilled worker will be able to prepare, test and utilize functional variants of the antibodies disclosed herein, while appreciating that variants having the ability to block the activity of CD44-SLC1A2 fusion protein or SLC1A2 protein would fall within the scope of the present invention. As used in this context, ability to block the activity of CD44-SLC1A2 fusion protein or SLC1A2 means a functional characteristic ascribed to an anti-CD44-SLC1A2 fusion protein antibody or SLC1A2 antibody described herein.

A functional variant can include, for example, an antibody that has at least one altered complementarity determining region (CDR) (hyper-variable) and/or framework (FR) (variable) domain/position, vis-a-vis a peptide sequence disclosed herein. To better illustrate this concept, a brief description of antibody structure follows.

An antibody is composed of two peptide chains, each containing one (light chain) or three (heavy chain) constant domains and a variable region (VL, VH), the latter of which is in each case made up of four FR regions and three interspaced CDRs. The antigen-binding site is formed by one or more CDRs, yet the FR regions provide the structural framework for the CDRs and can also play an important role in antigen binding. By altering one or more amino acid residues in a CDR or FR region, the skilled worker routinely can generate mutated or diversified antibody sequences, which can be screened against the antigen, for new or improved properties, for example.

It is preferred that variants are constructed by changing amino acids within one or more CDR regions; a variant might also have one or more altered framework regions. Alterations also may be made in the framework regions. For example, a peptide FR domain might be altered where there is a deviation in a residue compared to a germline sequence.

SLC1A2 gene encodes a membrane-bound protein which functions as the principal transporter clearing excitatory glutamate from extracellular space. Accordingly, as proteins encoded by SLC1A2 acts as channel protein, small molecule inhibitors may also be used to inhibit the activity of CD44-SLC1A2 fusion protein. Small molecule inhibitors that may be useful in the present invention includes, but are not limited to, cis-1-Amino-cyclobutane-1,3-dicarboxylic acid, (2S,1′S,2′R)-2-(Carboxycyclopropyl)glycine, 6,6′-[(3,3-Dimethoxy-[1,1′-biphenyl]-4,4′-diyl)bis(azo)]bis[4-amino-5-hydroxy-1,3-napthalene-disulphonic acid]tetrasodium salt, 7-Chloro-4-hydroxyquinoline-2-carboxylic acid or sodium salt derivatives thereof, (2S,3S,4R)-2-Carboxy-4-isopropyl-3-pyrrolidine acetic acid, 6,6-[(3,3′-Dimethyl-[1,1′-biphenyl]-4,4′-diyl)bis(azo) bis[4-amino-5-hydroxy-1,3-aphthalenedisulphonic acid]tetrasodium salt, (±)-3-Hydroxy-4,5,6,6a-tetrahydro-3a-H-pyrrolo[3,4-d]isoxazole-4-carboxylic acid, (±)-3-Hydroxy-4,5,6,6a-tetrahydro-3aH-pyrrolo[3,4-d]isoxazole-6-carboxylic acid, L-threo-b-Hydroxyaspartic acid, (±)-threo-3-Methylglutamic acid, L-anti-endo-3,4-Methanopyrrolidinedicarboxylic acid, L-trans-Pyrrolidine-2,4-dicarboxylic acid, (2S,4R)-4-Methylglutamic acid, DL-threo-β-Benzyl-oxyaspartic acid, (3S)-3-[[3-[[4-(Tri-fluoromethyl)benzoyl]amino]phenyl]methoxy]-L-aspartic acid, 2-Amino-5,6,7,8-tetra-hydro-4-(4-methoxyphenyl)-7-(naphthalen-1-yl)-5-oxo-4H-chromene-3-carbonitrile, and N-[4-(2-Bromo-4,5-difluorophenoxy)phenyl]-L-asparagine.

Apart from antibodies, peptide aptamers, anticalines, aptamers and spiegelmer as described in the prior art can be generated and used, respectively, for the various purposes disclosed herein for antibodies.

Typically, the inhibitors of CD44-SLC1A2 fusion gene or a functional variant thereof, or an inhibitor for a fusion protein expressed by the CD44-SLC1A2 fusion gene or the functional variant thereof are formulated into pharmaceutical compositions using techniques and procedures well known in the art. Pharmaceutically acceptable compositions are prepared in view of approvals for a regulatory agency or other agency prepared in accordance with generally recognized pharmacopeia for use in animals and in humans. The formulation should suit the mode of administration.

The inhibitors described herein can be provided as a pharmaceutical preparation in liquid form as solutions, syrups or suspensions. In liquid form, the pharmaceutical preparations can be provided as a concentrated preparation to be diluted to a therapeutically effective concentration before use. Generally, the preparations are provided in a dosage form that does not require dilution for use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). In another example, pharmaceutical preparations can be presented in lyophilized form for reconstitution with water or other suitable vehicle before use.

The pH of the stable formulations is important in retaining the structure and activity of the biologically active composition. Optimal pH can be obtained by formulation techniques known to those skilled in the art. For example, optimal pH can be determined by assessing aggregation and activity under differing pH conditions using various methods known to one of skill in the art, for example. Such assays or assessment include, but are not limited to, size exclusion chromatography, HSPEC determinations, heat stability data, anti complement titers of the various preparations and/or hyaluronidase activity assays. Typically, in the formulations provided herein the pH can range from 4.0 to 8.0 as measured in the concentrated solution of the formulation. Generally, within this range, a lower pH is desired, however, to ensure maximum monomer content. Accordingly, the formulations provided herein typically have a pH that is at least or about 4.0 to 7.4, generally at least or about 4.0 to 6.0, and typically 4.4 to 4.9. As noted, the indicated pH is measured in the concentrated solution of the formulation. pH can be adjusted using acidifying agents to lower the pH or alkalizing agents to increase the pH. Exemplary acidifying agents include, but are not limited to, acetic acid, citric acid, sulfuric acid, hydrochloric acid, monobasic sodium phosphate solution, and phosphoric acid. Exemplary alkalizing agents include, but are not limited to, dibasic sodium phosphate solution, sodium carbonate, or sodium hydroxide.

Any buffer can be used in the preparation of the liquid formulation provided herein so long as it does not adversely affect the stability of the formulation, and supports the requisite pH range required. Examples of particularly suitable buffers include succinate, acetate, phosphate buffers, citrate, aconitate, malate and carbonate. Those of skill in the art, however, will recognize that formulations provided herein are not limited to a particular buffer, so long as the buffer provides an acceptable degree of pH stability, or “buffer capacity” in the range indicated. Generally, a buffer has an adequate buffer capacity within about 1 pH unit of its pK. Buffer suitability can be estimated based on published pK tabulations or can be determined empirically by methods well known in the art. The pH of the solution can be adjusted to the desired endpoint within the range as described above, for example, using any acceptable acid or base,

The pharmaceutical composition of the present invention may comprise an inhibitor of CD44-SLC1A2 fusion gene or a functional variant thereof, or an inhibitor for a fusion protein expressed by the CD44-SLC1A2 fusion gene or the functional variant thereof.

In another embodiment, the pharmaceutical composition may comprise an inhibitor of SLC1A2 gene or a functional variant thereof, or an inhibitor for a protein expressed by the SLC1A2 gene or the functional variant thereof, and a compound for cancer treatment.

As used herein, an agent for cancer treatment (used interchangeably with “anti-tumor agent, anti-cancer agent or anti-neoplastic agent”) refers to any agents, used in anti-cancer treatment. “Cancer treatment” or “anti-cancer treatment” refers to any kind of known treatment of cancer which aims at eliminating or removing cancer cells. The major modalities of cancer treatment or therapy are surgery, and radiation therapy (for local and local-regional disease), and chemotherapy (for systemic disease). Other important methods include hormonal therapy (for selected cancers, such as prostate cancer, breast cancer or endometrium), immunotherapy (monoclonal antibodies, interferons, and other biologic response modifiers and tumor vaccines), the use of differentiating agents, such as retinoids, agents that exploit the growing knowledge of cellular and molecular biology and mixtures of the aforementioned treatments or therapies. The inhibitors of CD44-SLC1A2 fusion gene, or SLC1A2 gene, or CD44-SLC1A2 fusion protein, or SLC1A2 protein or functional variants of these genes and proteins of the present invention can be used in combination with any known cancer treatment and anti-cancer agent used in those known cancer treatments described herein.

Anti-cancer agents which are used in such cancer treatments can alleviate, reduce, ameliorate, prevent, or place or maintain in a state of remission of clinical symptoms or diagnostic markers associated with neoplastic disease, tumors and cancer, and can be used together with the inhibitors described herein in methods, combinations and compositions provided herein.

Exemplary anti-cancer agents include, but are not limited to cytokines, growth factors, hormones, photosensitizing agents, radionuclides, toxins, anti-metabolites, signaling modulators, anti-cancer antibiotics, anti-cancer antibodies, anti-cancer oligopeptides, angiogenesis inhibitors, radiation therapy, hypothermia therapy, hyperthermia therapy, laser therapy, chemotherapeutic compounds, or a combination thereof.

Chemotherapeutic compounds include, but are not limited to platinum; platinum analogs; anthracenediones; vinblastine; alkylating agents; alkyl sulfonates; aziridines; ethylenimines and methylamelamines; nitrosureas; antibiotics; anti-metabolites; folic acid analogues; androgens; anti-adrenals; folic acid replenisher; aminolevulinic acid; amsacrine; bestrabucil; bisantrene; edatraxate; defofamine; demecolcine; diaziquone; elformithine; elliptinium acetate; etoglucid; gallium nitrate; substituted ureas; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; anti-cancer polysaccharides; polysaccharide-K; razoxane; sizofuran; spirogermanium; tenuazonic acid; triaziquone; 2,2′,2″-trichlorotriethylamine; urethan; vindesine; dacarbazine; manno-mustine; mitobronitol; mitolactol; pipobroman; gacytosine; cytosine arabinoside; cyclophosphamide; thiotepa; taxoids, such as paclitaxel and doxetaxel; chlorambucil; gemcitabine; 6-thioguanine; mercaptopurine; methotrexate; etoposide (VP-16); ifosfamide; mitomycin C; vincristine; vinorelbine; navelbine; novantrone; teniposide; daunomycin; aminopterin; xeloda; ibandronate; CPT-1; topoisomerase inhibitor RFS 2000; difluoro-methylomithine (DMFO); retinoic acid; esperamicins; capecitabine; methylhydrazine derivatives; and pharmaceutically acceptable salts, acids or derivatives of any of the above.

Chemotherapeutic compounds also include, but are not limited to Cisplatin, adriamycin, non-sugar containing chloroethylnitrosoureas, bleomycin, doxorubicin, taxol, fragyline, Meglamine GLA, valrubicin, carmustaine and poliferposan, MM 1270, BAY 12-9566, RAS farnesyl transferase inhibitor, farnesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, IncelNX-710, VX-853, ZDOlOl, IS1641, ODN 698, TA 2516/Marmistat, BB2516/Marmistat, CDP 845, D2163, PD1 83805, DX8951f, Lemonal DP 2202, FK 317, Picibanil/OK-432, AD 32Nalrubicin, Metastron/strontium derivative, Temodal/Temozolomide, Evacet/liposomal doxorubicin, Yewtaxan/Placlitaxel, Taxol®/Paclitaxel, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, Iodine seeds, CDK4 and CDK2 inhibitors, PARP inhibitors, D4809/Dexifosamide, Ifes/Mesnex/Ifosamide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Anastrozole, Asparaginase, Busulfan, Carboplatin, Chlorambucil, Cladribine, Cytarabine HCl, Dactinomycin, Daunorubicin HCl, Denileukin diftitox, Estramustine phosphate sodium, Etoposide (VP 16-213), Exemestane, Floxuridine, Flutamide, Hydroxyurea (hydroxycarbamide), Ifosfamide, Interferon Alfa-2a, Interferon Alfa-2b, Interferon Gamma-Ib, Letrozole, Leuprolide acetate (LHRH-releasing factor analogue), Lomustine (CCNU), Mechlorethamine HCl (nitrogen mustard), Megestrol, Mercaptopurine, Mesna, Mitotane (o.p′-DDD), Mitoxantrone HCl, Octreotide, Pegaspargase, Plicamycin, Procarbazine HCl, Streptozocin, Tamoxifen citrate, Thioguanine, Thiotepa, Tretinoin, Vinblastine sulfate, Amsacrine (m-AMSA), Azacitidine, Erythropoietin, Hexamethylmelamine (HMM), Interleukin 2, Mitoguazone (methyl-GAG; methyl glyoxal bis-guanylhydrazone; MGBG), Pentostatin (2′ deoxycoformycin), Semustine (methyl-CCNU), Teniposide (VM-26®), Vindesine sulfate, Altretamine, Carmustine, Estramustine, Gemtuzumab ozogamicin, Idarubicin, Ifosphamide, Isotretinoin, Leuprolide, Melphalan, Testolactone, Uracil mustard, and the like. Also included in this definition are anti-hormonal agents that act to regulate or inhibit hormone action on tumors such as anti-estrogens, adrenocortical suppressants, antiandrogens and pharmaceutically acceptable salts, acids or derivatives of any of the above. Such chemotherapeutic compounds that can be used herein include compounds whose toxicities preclude use of the compound in general systemic chemotherapeutic methods. In one example of the present invention, the chemotherapeutic agent 5-fluorouracil or prodrugs thereof is excluded. Exemplary excluded prodrugs include, but are not limited to, Capecitabine/Xeloda® and UFT (tegafur/uracil). Exemplary chemotherapeutics agents that may be used in the present disclosure include cisplatin, adriamycine (doxorubicine), nutlin-3 and etoposide phosphate. The chemotherapeutic agent used in the present disclosure may be cisplatin.

The present invention also provides for a method of identifying a compound that modulates expression of CD44-SLC1A2 fusion gene in a cell. Such a method may comprise or consist of:

(a) exposing cells expressing CD44-SLC1A2 fusion gene with a test compound;
(b) determining the expression level of CD44-SLC1A2 fusion gene in the cells which were exposed to the test compound under (a);
(c) comparing the level of expression of the CD44-SLC1A2 fusion gene determined under (b) with the expression of the CD44-SLC1A2 fusion gene in control cells which were not exposed to the test compound; wherein a difference in the expression level between the cells under (b) compared to the control cells identifies the compound that modulates expression of CD44-SLC1A2 fusion gene in a cell.

In another embodiment, there is provided a method of identifying a compound that modulates the amount of a fusion protein encoded by CD44-SLC1A2 fusion gene comprised in a cell. Such a method can comprise or consist of:

(a) exposing cells expressing the fusion protein with a test compound;
(b) determining the amount of the fusion protein in the cells which were exposed to the test compound under (a);
(c) comparing the amount of CD44-SLC1A2 fusion protein determined under (b) with the amount of CD44-SLC1A2 fusion protein in control cells not exposed to the test compound; wherein a difference in the amount of the CD44-SLC1A2 fusion protein between the cells under (b) compared to the control cells identifies the compound that modulates the amount of CD44-SLC1A2 fusion protein in the cells.

CD44-SLC1A2 fusion gene, or CD44-SLC1A2 fusion protein, or SLC1A2 gene can also be used as biomarker for diagnostic purposes. In one aspect, there is provided a prognostic method for determining the clinical outcome of a patient with gastrointestinal cancer. The prognostic method comprises identifying and determining the level of expression of SLC1A2 gene, or functional variants thereof in the patient, wherein an increased SLC1A2 expression, indicates that the patient may have an improved chance of survival. A patient with reduced SLC1A2 expression indicates that the patient may have a lower chance of survival.

In another embodiment, the prognostic method comprises identifying and determining the presence and/or level of expression of CD44-SLC1A2 fusion gene, or functional variants thereof in the patient, wherein the presence or increased expression of CD44-SLC1A2 fusion gene indicates that the patient have lower chance of survival.

Increased SLC1A2 expression may be determined by comparing the expression level of SLC1A2 in a tumor against a reference tumor obtained from a patient known to have gastrointestinal cancer and that the tumor is known to have increased SLC1A2 expression. In one example, patients with increased expressing SLC1A2 includes any tumors with SLC1A2 expression that deviates from a reference tumor only by 10% or 8% or 5% or 4% or 3% or 2% between 1 and 5%. Another reference that may be used to determine increased SLC1A2 expression is by comparing the level of expression of SLC1A2 to a pre-existing gastrointestinal tumor database. High SLC1A2 expression, when determined using pre-existing database, includes the expression level of the top 15% or 10% or 8% or 5% or 4% or 3% or 2% between 1 and 5%. of the SLC1A2 expressing tumors.

In contrast for the fusion gene CD44-SLC1A2 an increased expression means a lower chance of survival. A reference for determining an increased expression level could be a reference cell known to be a cancerous cell (tumor cell). In that case a deviation in the expression level of CD44-SLC1A2 from the expression level in the cancerous cell of only 10% or 8% or 5% or 4% or 3% or 2% between 1 and 5%. would indicate an increased expression level for CD44-SLC1A2 and thus an increased risk to develop cancer.

The term “improved survival”, as used herein refers to a prediction that indicates the likelihood of a patient surviving more than 5 years post diagnosis. The term “lower (decreased) chance of survival”, as used here in refers to a prediction that indicates the likelihood of a patient not surviving more than 5 years post diagnosis.

By determining the presence or absence of such a biomarker it is also possible to determine whether a patient is at risk of developing a gastrointestinal cancer. Thus, in one aspect the present invention is directed to a method of assessing the risk of a patient to develop gastrointestinal cancer. The method comprises or consists of identifying the presence of a CD44-SLC1A2 fusion protein or the presence of a CD44-SLC1A2 fusion gene or both or functional variants thereof in a patient wherein the presence of any of these genes or proteins indicates that the person is at risk of developing gastrointestinal cancer or suffers from gastrointestinal cancer.

According to the methods of present invention, inhibitors described herein, pharmaceutical compositions comprising those inhibitors and pharmaceutical compositions administered together with inhibitors described herein (in the following it is simply referred to inhibitors for all these alternatives) may be administered by any suitable route, either systemically, regionally or locally. The particular route of administration to be used in any given circumstance will depend on a number of factors, including the nature of the gastrointestinal cancer to be treated, the severity and extent of the gastrointestinal cancer, the required dosage of the particular inhibitors to be delivered and the potential side-effects of the desired inhibitors.

For example, in circumstances where it is required that appropriate concentrations of the desired inhibitors are delivered directly to the site to be treated, administration may be regional rather than systemic. Regional administration provides the capability of delivering very high local concentrations of the desired inhibitors to the required site and thus is suitable for achieving the desired therapeutic or preventative effect whilst avoiding exposure of other organs of the body to the inhibitors and thereby potentially reducing side effects.

By way of example, administration according to embodiments of the invention may be achieved by any standard routes, including intracavitary, intravesical, intramuscular, intraarterial, intravenous, subcutaneous, topical or oral. Intracavitary administration may be intraperitoneal or intrapleural.

If desired, devices or compositions containing expression cassettes as described herein, suitable for sustained or intermittent release could be, in effect, implanted in the body or topically applied thereto for the relatively slow release of such materials into the body.

Administration of an expression vector or host cell may include delivery via direct oral intake, systemic injection, or delivery to selected tissue(s) or cells, or indirectly via delivery to cells isolated from a subject or a compatible donor.

With regard to compositions comprising nucleic acid based inhibitors, such as the interfering ribonucleic acids described herein, all modes of delivery of such compositions are contemplated by the present invention. Delivery of these compositions to cells or tissues of an animal may be facilitated by microprojectile bombardment, liposome mediated transfection (e.g., lipofectin or lipofectamine), electroporation, calcium phosphate or DEAE-dextran-mediated transfection, for example. In an alternate embodiment, a synthetic construct may be used as a therapeutic or prophylactic composition in the form of a “naked DNA” composition as is known in the art. The compositions may be administered by intradermal (e.g., using Panjet™ delivery) or intramuscular routes.

The step of introducing the synthetic polynucleotide into a target cell will differ depending on the intended use and species, and can involve one or more of non-viral and viral vectors, cationic liposomes, retroviruses, and baculoviruses. Such methods can include, for example:

Local application of the nucleic acid based inhibitor by injection, surgical implantation, instillation or any other means. This method can also be used in combination with local application by injection, surgical implantation, instillation or any other means, of cells.

General systemic delivery by injection of nucleic acids, alone or in combination with liposomes, viral capsids or nanoparticles or any other mediator of delivery. Improved targeting might be achieved by linking the synthetic nucleic acid to a targeting molecule (the so-called “magic bullet” approach), or by local application by injection, surgical implantation or any other means, of another factor or factors required for the activity of the nucleic acid.

The effective dose level of the administered inhibitor for any particular subject will depend upon a variety of factors including: the type of gastrointestinal cancer being treated and the stage of the gastrointestinal cancer; the activity of the inhibitor employed; the composition employed; the age, body weight, general health, sex and diet of the subject; the time of administration; the route of administration; the rate of sequestration of compounds; the duration of the treatment; drugs used in combination or coincidental with the treatment, together with other related factors well known in the art.

One skilled in the art would be able, by routine experimentation, to determine an effective, non-toxic dosage which would be required to treat applicable conditions. These will most often be determined on a case-by-case basis.

In terms of weight, a therapeutically effective dosage of a composition for administration to a patient is expected to be in the range of about 0.01 mg to about 150 mg per kg body weight per 24 hours; typically, about 0.1 mg to about 150 mg per kg body weight per 24 hours; about 0.1 mg to about 100 mg per kg body weight per 24 hours; about 0.5 mg to about 100 mg per kg body weight per 24 hours; or about 1.0 mg to about 100 mg per kg body weight per 24 hours. More typically, an effective dose range is expected to be in the range of about 5 mg to about 50 mg per kg body weight per 24 hours.

Alternatively, an effective dosage may be up to about 5000 mg/m². Generally, an effective dosage is expected to be in the range of about 10 to about 5000 mg/m², typically about 10 to about 2500 mg/m², about 25 to about 2000 mg/m², about 50 to about 1500 mg/m², about 50 to about 1000 mg/m², or about 75 to about 600 mg/m².

Further, it will be apparent to one of ordinary skill in the art that the optimal quantity and spacing of individual dosages will be determined by the nature and extent of the condition being treated, the form, route and site of administration, and the nature of the particular individual being treated. Also, such optimum conditions can be determined by conventional techniques.

It will also be apparent to one of ordinary skill in the art that the optimal course of treatment, such as, the number of doses of the inhibitor given per unit time, can be ascertained by those skilled in the art using conventional course of treatment determination tests.

The present invention also provides kits for use in treating or preventing gastrointestinal cancer in a patient, wherein the kit may include the isolated nucleic acid, vector, host cell, isolated fusion protein, antibodies or pharmaceutical composition as described herein.

In the context of the present invention, a compartmentalized kit includes any kit in which reagents are contained in separate containers, and may include small glass containers, plastic containers or strips of plastic or paper. Such containers may allow the efficient transfer of reagents from one compartment to another compartment whilst avoiding cross-contamination of the samples and reagents, and the addition of agents or solutions of each container from one compartment to another in a quantitative fashion.

Typically, a kit of the present invention will also include instructions for using the kit components to conduct the appropriate methods.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1. CD44-SLC1A2 expression in primary gastric cancer tumors. (A) CD44-SLC1A2 RT-PCR on two index primary gastric cancer samples (GC980417 and GC2000038) with SLC1A2 genomic breakpoints (see FIG. 3). GN2000038 is the matched normal sample to GC2000038. Fusion positive SNU16 cells are included as a positive control. (B) CD44-SLC1A2 RT-PCR on 43 gastric tumors and matched normal gastric tissues. (top) Tumors. Stars highlight CD44-SLC1A2 expressing tumors (GC980390, GC2000639). (bottom) Matched normals. SNU16 is included as a positive control (C) Sequence of the CD44-SLC1A2 fusion junction in GC2000038. Black bars indicate the fusion junction. (D) Long-range genomic PCR analysis. Primers used are the same as FIG. 4F. GC2000038 and GC980390 are fusion-positive primary GCs. GN2000038 and GN980390 are matched normal controls.

FIG. 2. MYC, ERBB2, RAB23, and PTEN genomic aberrations in SNU16, N87, HS746T and TMK1 cells detected by high density array-based comparative genomic hybridization microarrays. From left to right: 1) Copy number amplification of the MYC oncogene detected in SNU16 cells 2) ERBB2 amplification in N87 gastric cancer cells 3) RAB23 amplification in HS746T gastric cancer cells 4) PTEN deletion in TMK1 cells. X-axis, log 2-transformed smoothened copy number values averaged over a 0.1 Mb moving window. Y-axis, physical genomic coordinates along chromosome. X-axis: log transformed smoothened copy number value. The black arrows point to the respective genes. Vertical dashed lines indicate copy number values of 2 (Log-transformed copy number=0).

FIG. 3. Genomic breakpoint analysis of gastric cancer. (A) Representative example of a genomic breakpoint. aCGH profile of GC tumor GC20020700 exhibiting a genomic breakpoint in the CALCR gene on Chr 7q12. X-axis—physical chromosomal coordinates. Y-axis—log 2-transformed smoothened values (i.e. 0 indicates copy number equals to 2). The black arrow indicates the breakpoint of interest. Each dot represents a microarray probe. (B) Genomic breakpoints in the 5′ region of SLC1A2 in four GCs (3 primary tumors and 1 cell line—GC2000038, GC980417, GC20021048, SNU16). Black arrows indicate the breakpoints of interest. (C) FISH Validation of SLC1A2 breakpoints. Probes WI2-67019 (left panel) and WI2-1928P9 (right panel) cover SLC1A2 exon 1 or intron 1 respectively. (D) Genomic organization of the SLC1A2 gene. Vertical bars represent SLC1A2 exons, connected by intervening introns. Total length of the SLC1A2 gene is 168 Kb. Black bar: minimal common recurrent breakpoint region in SLC1A2 intron 1 (15-24 kb). Black arrow at bottom: location of oligonucleotide primer used for 5′ RLM-RACE analysis.

FIG. 4. CD44-SLC1A2

Gene Fusions. (A) 5′ SLC1A2 RLM-RACE of GC cell lines. (B) CD44-SLC1A2 fusion sequence. No underline: CD44 exon 1. Underlined: SLC1A2 exon 2. ATG sites are underlined. (C) CD44-SLC1A2 RT-PCR. Primers were targeted to CD44 exon 1 (top horizontal arrow) and SLC1A2 exons 3, 4, 5, 6 (bottom vertical arrows). NG—normal stomach, N87—fusion negative line (D) (top) CD44 and SLC1A2 chromosomal organization (bottom) CD44-SLC1A2 relationship to CD44 and SLC1A2 parent genes. (E) Fiber-FISH. (left) Probe 1 (Rp1-68d18) covers CD44 (3′ of intron 1) and SLC1A2 (3′ of intron 1). Probe 2 (Rp11-1148123) covers the 5′ region of SLC1A2 intron 1 and upstream sequence. (right) Fiber-FISH images of control CCL159 cells and fusion-positive SNU16 cells. (F) Long-range PCR. Primers were targeted to CD44 exon 1 and the SLC1A2 1st intron (black arrows in (E)). SNU16—fusion positive. AGS—fusion negative. Primers are black arrows in (E). (G) Western blot of fusion-positive SNU16 and fusion-negative AGS and SNU5 cells (membrane fractions). (top) anti-SLC1A2 antibodies. (bottom) α-tubulin control. (H) CD44-SLC1A2 ectopic expression. (top) CD44-SLC1A2 expression construct carrying a GFP tag. Arrow—promoter. ATG sites in CD44 exon 1 and SLC1A2 exon 2 are shown. (bottom) Immunoblotting with anti-SLC1A2 antibodies.

FIG. 5. 3′RACE and full-length CD44-SLC1A2 expression in SNU16 cells. (A) Left, 3′RACE analysis of SLC1A2 identifies 2 kb products in both AGS and SNU16. Right, sequencing of the 3′ RACE products confirms wild-type full length SLC1A2 transcripts in both SNU16 and AGS cells. (B) RT-PCR screening of the CD44-SLC1A2 fusion in 42 gastric cancer cell line and gastric normal cell lines. SNU16 (lighter text) is the only cell line expressing the fusion. (C) Left, RT-PCR analysis using a forward primer to CD44 exon 1 and reverse primer to SLC1A2 exon 11 (last exon) confirms expression of a 1.6 kb CD44-SLC1A2 fusion transcript in SNU16 cells. Right, PCR product sequencing confirms a full length fusion in SNU16.

FIG. 6. Chromosomal inversion model of CD44-SLC1A2 gene fusion. (A) Inferred paracentric inversion model between CD44 and SLC1A2. Upper, BP1 and BP2 indicate the inferred breakpoint positions in CD44 and SLC1A2 respectively (Main Text). Curved arrows indicate the predicted paracentric inversion, resulting in formation of the CD44-SLC1A2 fusion. Horizontal bars represent the genomic coordinates of probes Rp1-68d18 and Rp11-1148123 respectively. Lower, predicted fiber-FISH patterns for both normal chromosome and chromosomal inversions using probes Rp1-68d18 and Rp11-1148123. (B) Spectral karyotyping (SKY) analysis of SNU16 cells reveals an unbalanced chromosomal rearrangement affecting 11p13-11p14. Left, SKY karyotype showing chromosome aberration t(1;11), indicating a rearrangement of chromosome 1 with chromosome 11 at bands 11p14. Right, SKY identifies the complex chromosomal aberration t(5;11;10) with rearrangements joining material from three different chromosome 5, 11, 10.

FIG. 7. Predicted protein structure of CD44-SLC1A2 and expression in primary gastric cancer samples. A) CD44-SLC1A2 expression patterns. Left, in the first pattern, translation initiates from an ATG site in CD44 exon 1, producing a 65aa protein. Right, in the second pattern, translation initiates from an alternative ATG site in SLC1A2 exon 2, producing a truncated SLC1A2 protein of 565aa. The lower diagram illustrates the known protein domains of full length SLC1A2. The black arrow indicates the position of second protein translation initiation site. A truncated SLC1A2 protein beginning from exon 2 is predicted to encode all known functional domains. B) Cloning and sequencing of CD44/SLC1A2 RT-PCR products from two fusion positive tumors (GC980390 and GC2000639) confirm a fusion between CD44 exon 1 and SLC1A2 exon 2. Black bars indicate the fusion junction.

FIG. 8. CD44-SLC1A2 fusions enhance cellular proliferation, colony formation and invasion. (A) CD44-SLC1A2 silencing by fusion-specific siRNA1 (CGCAGAUCGUGCCAACAAUUU). CD44-SLC1A2 expression was measured after 24 h, 48 h and 72 h post siRNA treatment. CD44:wild-type CD44 expression. CD44 primers were designed to target exons 3-5. SLC1A2:wild-type SLC1A2 expression. SLC1A2 primers were designed to target exon 1. GADPH was used as a loading control. (B) Western blotting. SLC 1A2 protein levels were monitored using anti-SLC1A2 antibodies. α-tubulin is used as a loading control. − and +: SNU16 cells pre (−) and post (+) treatment with fusion-specific siRNAs. (C)-(E) Effects of CD44-SLC1A2 knockdown. (C) Proliferation rates of SNU16 cells before and after CD44-SLC1A2 siRNA treatment. (D) Colony formation assays using SNU16 cells before and after CD44-SLC1A2 siRNA treatment. (E) Cell invasion assays using SNU16 cells before and after CD44-SLC1A2 siRNA treatment. (F)-(G) Effects of CD44-SLC1A2 overexpression. (F) Cell proliferation rates of HFE145 cells before and after CD44-SLC1A2 overexpression (G) Colony formation assays using HFE145 cells before and after CD44-SLC1A2 overexpression (H) Cell invasion assays using HFE145 cells before and after CD44-SLC1A2 overexpression. All experiments were performed in triplicate. P-values were computed using Student's t-test. Red stars represent p-values exceeding the significance threshold (P<0.05).

FIG. 9. Silencing CD44-SLC1A2 with a second fusion specific siRNA inhibits cellular proliferation, invasion, and colony formation. (A) Specific silencing of CD44-SLC1A2 by fusion-specific siRNA2 (GCACAUCGUGCCAACAAUAUU). Reduction of CD44-SLC1A2 expression was observed after siRNA2 treatment, without impairing regular CD44 and SLC1A2 expression. (B) Western blotting analysis confirms knockdown of SLC1A2 protein expression in SNU16 (membrane fraction). An α-tubulin control confirms equal amount of protein loading in both lanes. (C) Immunofluorescence analysis of control and fusion silenced SNU16 cells using SLC1A2 antibodies. Untreated SNU16 cells express strong membranous expression of SLC1A2, which is significantly reduced by siRNA treatment. (D) SNU16 cells treated with CD44-SLC1A2 siRNA2 exhibit a significant decrease in proliferation rate (p<0.001) compared to control siRNA treated cells. (D) Colony formation assays reveal a reduction in numbers of colonies formed by fusion specific siRNA2 treatment compared to scrambled siRNA treatment. (E) Knockdown of CD44-SLC1A2 using fusion siRNA2 also results in a significant reduction of invasion rate (p=0.04).

FIG. 10. CD44-SLC1A2 silencing does not affect AGS cells. (A) Left, expression of CD44-SLC1A2, SLC1A2 and CD44 after CD44-SLC1A2 siRNA1 treatment in AGS fusion negative cells. Right, proliferation of AGS cells is not affected by fusion-specific siRNA silencing. (B) Left, expression of CD44-SLC1A2, SLC1A2 and CD44 after CD44-SLC1A2 siRNA2 treatment in AGS fusion negative cells. Right, proliferation of AGS cells is not affected by fusion-specific siRNA silencing.

FIG. 11. Reduction of cellular proliferation in fusion-negative AGS cells after silencing of wild-type SLC1A2 (A) Silencing of wild-type SLC1A2 in AGS cells using a siRNA targeting wild-type SLC1A2. Upper, reduction of SLC1A2 protein expression after SLC1A2 siRNA treatment. An α-tubulin control confirms equal amount of protein loading in both lanes. Lower, AGS cell proliferation after treatment with a scrambled siRNA control or SLC1A2 siRNA1. (B) AGS treated with a second non-overlapping siRNA targeting SLC1A2.)

FIG. 12. CD44-SLC1A2 Regulates Intracellular Glutamate Levels and Sensitizes Cells to Cisplatin. (A) Glutamate levels in primary GCs compared to matched normal controls. x-axis: 20 cancer/normal pairs (Gastric cancer—black, Matched normal—grey). y-axis:glutamate concentration. P-values were computed using a paired t-test. (B) Glutamate levels in GC cells before and after CD44-SLC1A2 siRNA treatment. All experiments were performed in triplicate. (C) Cisplatin sensitivity of SNU16 cells with and without stable CD44-SLC1A2 siRNA silencing. All experiments were performed in triplicate. P-values were computed at 10 μM cisplatin treatment. P-values for (B) and (C) were computed using a t-test. Stars depict p-values exceeding the significance threshold (P<0.05).

FIG. 13. CD44-SLC1A2 knockdown does not Sensitize Cells to 5-Fluorouracil Chemotherapy. 5-Fluorouracil sensitivity of SNU16 cells before and after CD44-SLC1A2 siRNA1 silencing. There is no significant change in sensitivity to 5-Fluorouracil after CD44-SLC1A2 siRNA treatment compared to treatment with a scrambled siRNA control (p=0.33).

FIG. 14. CD44-SLC1A2 positive tumors are associated with high SLC1A2 expression Graph: x-axis—197 gastric cancer samples sorted by levels of SLC1A2 expression. Gene expression data were median-centered. The top 15% of high SLC1A2 expressing tumors are shown in on the right. (inset) RT-PCR screening of CD44-SLC1A2 in the top 15% of high SLC/A2-expressing gastric cancer samples. GADPH was used as a loading control. Samples expressing CD44-SLC1A2 are highlighted using crosses. The smaller band of 200 bp was sequenced and identified to be nonspecific (ns). CD44-SLC1A2 RT-PCR analysis of the matching 15 normal gastric tissues is also shown.

FIG. 15. Unsupervised clustering of gastric cancer samples expression profiles reveals clustering of SLC1A2-High Expressing Tumors. 197 gastric cancer samples gene expression profiles were clustered using an unsupervised hierarchical clustering algorithm. Samples with high SLC1A2 expression (top 15%) are indicated with a ‘+’. Several of the high SLC1A2 expressing tumors are observed to cluster close to one another (black bar).

FIGS. 16 A1, A2, B1 and B2 11p13 copy number status in CD44-SLC1A2 expressing samples. Seven CD44-SLC1A2 expressing tumors were analyzed for 11p13 copy number amplification using Affymetrix SNP6 arrays. The X-axis indicates genomic position and the y-axis is the log transformed copy number data. Each dot represents a SNP array probe. Segmented copy number data are plotted as horizontal black lines. The CD44 and SLC1A2 gene region is highlighted as a rectangle. (A1-2) Two fusion-positive samples (GC980390 (A1) and GC990172T (A2)) exhibiting 11p13 amplification. (B1-2) Two fusion-positive samples (GC2000114 (B1) and GC990090 (B2)) without 11p13 amplification.

FIG. 17. CD44 and SLC1A2 Expression levels of in 11p13 non-amplified, 11p13 amplified, and Fusion Positive samples. (A) and (B) CD44 and SLC1A2 expression in 45 gastric tumor samples profiled on both gene expression and SNP microarrays. Of these 45, 32 samples are 11p13 non-amplified (fusion neg, 11p13 Amp neg), 6 samples are 11p13 amplified but fusion-negative (fusion neg, 11p13 Amp pos), and 7 samples are CD44-SLC1A2 fusion positive (Fusion pos). Significant p-values (P<0.05, Student's t-test) are shown. (A) SLC1A2 gene expression measured using Affymetrix probe 225491_at. Fusion positive samples show significantly increased SLC1A2 gene expression compared to non-amplified samples (P=0.004), but 11p13 amplified samples do not (P=0.86). The arrow highlights one 11p13 amplified sample (GC980417) with a high-level focal amplification in the CD44/SLC1A2 gene region, suggesting that in some cases 11p13 amplification can drive increased SLC1A2 gene expression. (B) CD44 gene expression measured using Affymetrix probe 212063_at. 11p13-amplified samples show significantly increased CD44 expression compared to non-amplified samples (P=0.016) but fusion positive samples show a significantly decreased level of CD44 expression (P=0.0059), consistent with the CD44/SLC1A2 inversion event decoupling the wild-type CD44 gene from its endogenous promoter. (C)-(D) Real-time PCR analysis of CD44-SLC1A2 expression (targeting the fusion junction), and SLC1A2 targeting SLC1A2 exon 1). The tumors are Grp 1:T1-3, 11p13 non-amplified and fusion negative; Grp 2:T4-6, 11p13 amplified and fusion negative; and Grp 3:T7-9:11p13 non-amplified but fusion positive. AGS (fusion-negative) and SNU16 (11p13 amplified, fusion positive) were also included. All readings were normalized against a GADPH housekeeping control. Each data point represents the average of two independent experiments. (C) CD44-SLC1A2 expression. Only Grp 3 fusion-positive tumors and SNU16 cells are observed to express CD44-SLC1A2. (D) SLC1A2 exon 1 expression. 11p13-amplified samples are observed to express high levels of SLC1A2 exon1, consistent with these samples exhibiting “copy number driven” expression. In contrast, SLC1A2 exon 1 is not highly expressed in 2 out of 3 fusion positive samples, consistent with the high levels of 3′ SLC1A2 transcript in these samples being driven by fusion to CD44.

FIG. 18 SLC1A2 expression in 197 primary gastric tumors is prognostic of improved survival. Gastric cancer patients were ranked according to SLC1A2 expression levels, and overall survival of patients with high expression (top 15%) was compared to the remaining patients. Kaplan-Meier plots are shown. Log-rank test was used to compare the survival curves between groups and to generate the p-values for these comparisons.

### EXAMPLES

Non-limiting examples of the invention, including the best mode, and a comparative example will be further described in greater detail by reference to specific Examples, which should not be construed as in any way limiting the scope of the invention.

Materials and Methods

Primary Tumors and Cell Lines

Primary gastric tumors were obtained from the Singhealth Tissue Repository, an institutional resource of National Cancer Centre of Singapore and Singapore General Hospital. All patient samples were obtained with informed patient consent and approvals from Institutional Review Boards and Ethics Committees. Gastric cancer cell lines (GCCLs) AGS, KATO III, SNU1, SNU16, N87, and Hs746T were purchased from the American Type Culture Collection. AZ521, Ist1, TMK1, MKN1, MKN7, MKN28, MKN45, MKN74, Fu97, and IM95 cells were obtained from the Japan Health Science Research Resource Bank. SCH cells were provided by Yoshiaki Ito (Cancer Sciences Institute of Singapore). YCC cells were a gift from Sun-Young Rha (Yonsei Cancer Center, South Korea).

RNA and DNA Extraction

Genomic DNA from samples was extracted using a Qiagen Blood and Cell Culture DNA extraction kit. Total RNA was extracted using Qiagen RNA extraction reagents (Qiagen). Both RNAs and DNAs were quantitated using either a Nanodrop ND-1000 (Nanodrop Technologies) or Agilent Bioanlayzer 2100 (Agilent Technologies, Santa Clara, Calif.).

Genomic Breakpoint Analysis (GBA)

Genomic Breakpoint Analysis was performed on a panel of 106 primary tumors and 27 cell lines using Agilent 244K Human Genome Microarrays (Agilent Technologies, Santa Clara, Calif.). Sample labeling and hybridizations were performed according to the manufacturer's instructions. Tumor and control genomic DNAs (human spleen DNA) were labeled with Cy3-dUTP and Cy5-dUTP respectively. Hybridized slides were scanned on an Agilent DNA Microarray Scanner (Agilent Technologies) and images were extracted using Agilent Feature Extraction software. Data was analyzed using Agilent CGH Analytics software (v.3.5), using a Z-score algorithm with a threshold of 2.0 and a 1 pt window to identify genomic breakpoints.

Fluorescence In-Situ Hybridization (FISH)

SNU16 interphase and metaphase cells pellet was prepared for FISH analysis by standard hypotonic treatment and fixation after colcemide exposure (10 μg/ml) for 2 hrs. Prior to hybridization, cells were pre-treated with epsin (100 mg/ml) (Sigma) and 0.01 mol/L HCl at 37° C. (5 min), fixed in 1% formaldehyde (Sigma) (10 min), and dehydrated in an ethanol series. Fosmid and BAC probes were obtained from BACPAC Resource Center (BPRC, CHORI, Oakland, Calif., USA), grown following manufacturer's instructions. DNA was extracted with Nucleobond PC500 (Macherey-Nagel), followed by labeling with either biotin-16-dUTP (Roche) or digoxigenin-11-dUTP (Roche) using an Enzo Nick Translation DNA labeling system. Approximately 20 ng of each probe was used per hybridization in addition to 10 μg of Cot1-DNA (Invitrogen). The slides and probes mixes were codenatured on a hot plate set at 75° C. and hybridized overnight at 37° C. Post hybridization washes were performed at 45° C. in pre-warmed formamide 50%/2×SSC solution (twice), followed by two washes in 2×SSC (twice). Slides were blocked with blocking reagent (Roche), followed by incubation with avidin conjugated fluorescein isothiocyanate (FITC) (Roche) and anti-Digoxygenin-Rhodamine (Roche) respectively. DAPI counterstain (Vector Laboratories) was then used to stain the nuclei to enable visualization. Slides were then mounted with vectashield (Vector Laboratories). Fluorescence images were captured with 60× objective using a cooled charge-coupled device (CCD) camera attached to a Nikon 20 fluorescence microscope. Automated images capture was performed using ISIS software (Metasystems).

RLM-RACE (RNA-Ligase Mediated Rapid Amplification of cDNA Ends)

RLM-RACE was performed using the FirstChoice® RLM-RACE kit (Applied Biosystems). 10 mg of total RNA was first treated with Calf Intestinal Alkaline Phosphatase (CIP) to remove 5′ phosphate groups, followed by Tobacco Acid Pyrophosphatase to remove 5′ cap structures. After RNA linker ligation, mRNA transcripts were reverse transcribed using M-MLV reverse transcriptase. To amplify first strand cDNAs, outer 5′ PCR was performed using 5′ RACE outer primers and a SLC1A2 exon 3 primer (5′-ACACACTGCTCCCAGGATGA-3′ (SEQ ID NO: 1)) with SuperTaq™ Plus polymerase (Applied Biosystems). Subsequently, inner 5′ PCR was performed using a 5′ RACE inner primer (provided in kit) and a SLC1A2 exon 2 primer (5′-AGCCAAGATGACTGTCGTGCATTC-3′ (SEQ ID NO: 2)). After gel electrophoresis, PCR bands of interest were excised and cloned into pCR® 4-TOPO® (Invitrogen) vectors. Purified plasmid DNAs were sequenced bi-directionally on an ABI 3730 automated sequencer (Applied Biosystems). A minimum of 5 independent colonies were sequenced in each experiment.

cDNA Synthesis and Reverse-Transcription PCR (RT-PCR)

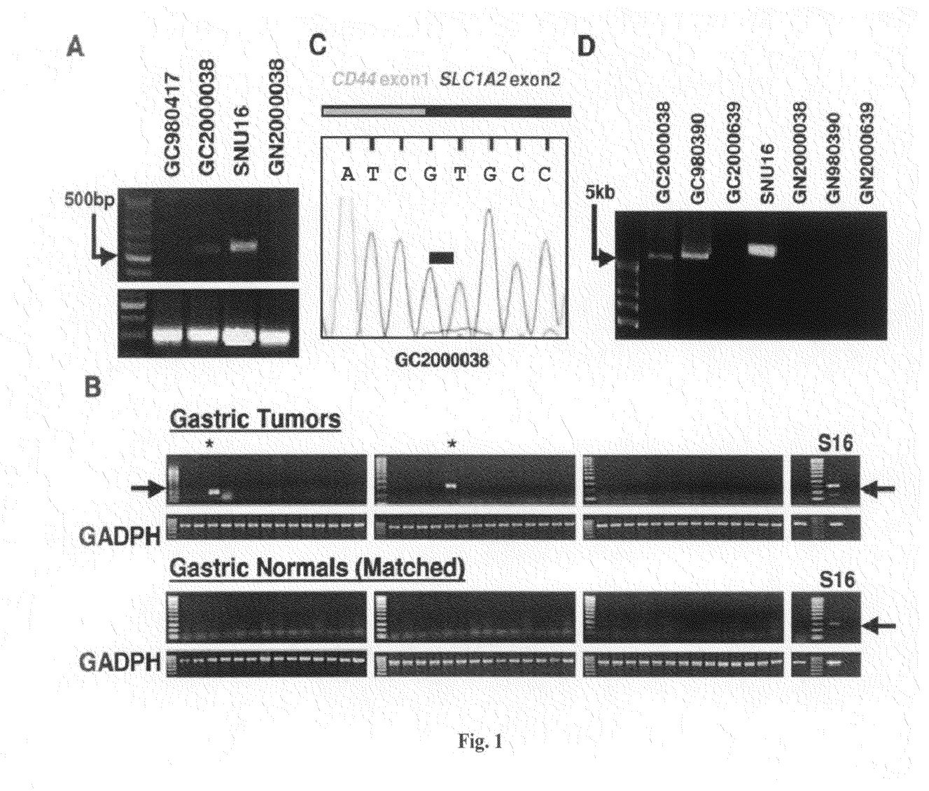

Gastric cancer RNAs were reverse transcribed by SuperScript II reverse transcriptase enzyme using oligo-dT (T18) primers (Invitrogen). To detect CD44-SLC1A2, RT-PCR was performed using forward primers to CD44 exon 1 (5′-CCATGGACAAGTTTTGGTGGCA-3′ (SEQ ID NO: 3)) and reverse primers to either SLC1A2 exon 3 (5′-GTATATCCCCTGGGAAGGCT-3′ (SEQ ID NO: 4)); exon4 (5′-CAGCTGCTTCTTGAGCTTGGGA-3′) (SEQ ID NO: 5); exons (5′-AAGCAGGCTTGGACAAGGTT-3′ (SEQ ID NO: 6)) or exon 6 (5′CTCGTTCAACAGAGAGACAACAGC-3′ (SEQ ID NO: 7)). Products were resolved by gel electrophoresis and bands of interest excised and cloned for subsequent analysis. To evaluate wild-type CD44 and SLC1A2 expression independently of CD44-SLC1A2, we used CD44 primers targeting exons 3, 4, 5; and SLC1A2 primers targeting exon 1. CD44-SLC1A2 RT-PCR involving clinical specimens (FIG. 1) was performed in an unselected cohort of gastric cancer patients. Reactions were repeated a minimum of three independent times.

DNA Sequencing

Purified PCR products were sequenced in forward and reverse directions using the ABI PRISM BigDye Terminator Cycle Sequencing Ready Reaction kit (Version 3) and ABI PRISM 3730 Genetic Analyzer (Applied Biosystems, CA). Chromatograms were analyzed by SeqScape V2.5 and manual review.

Fiber-FISH

SNU16 cells and control cells (normal lymphoblastoid CCL159) were grown in RPMI 1640 enriched with 15% FBS, 1% PS and 1% L-Glutamine. 2-3 ml of each cell suspension was centrifuged at 1200 rpm for 12 mins and then were washed with 6 ml PBS twice. Pellets were diluted with PBS to a final concentration around 2−3×104/ml. 10 μl of each cell suspension was spread on a poly-L-lysine (Sigma) coated slide, air dried and then fitted into a Cadenza Coverslip according to manufacturer's recommendations (Thermo Shandon). 150 μl of freshly made lysis solution (5:2 70 mM NaOH:absolute Ethanol) was applied to the slides, followed by 150 μl of 96% Ethanol. Slides were air dried at room temperature, treated with 3:1 Acetic Acid:Ethanol Fixative for 5 mins and dehydrated in Ethanol series (70%, 90%, and 100%) for 3 mins each. The FISH procedure was then applied.

Long Range Genomic PCR

CD44/SLC1A2 chromosomal inversions were detected using long range PCR kit (Qiagen) following the manufacturer's instructions. Reactions were performed using a forward primer at CD44 exon 1 (5′-GAAGAAAGCCAGTGCGTCTC-3′ (SEQ ID NO: 8), positive strand) and a reverse primer at SLC1A2 intron 1 in the minimal breakpoint region (5′-GAGGGCTGTCCTTAACGCCTAGC-3′ (SEQ ID NO: 9), negative strand). Experiments were repeated a minimum of three independent times.

Western Blotting

Cells were harvested in lysis buffer (10 mM Tris.Cl pH 7.5, 150 mM NaCl, 1% Triton X-114) for 1 hour at 40° C., and centrifuged at 800 g. Supernatants were incubated at 30° C. for 5-10 min and further centrifuged at RT, 300 g. Western blotting was performed on membrane fractions using the following antibodies and dilutions: 1:500 SLC1A2/EAAT2 (Cell Signaling Technologies), 1:2000 α-tubulin (Cell Signaling Technologies). Experiments were repeated a minimum of three independent times.

CD44-SLC1A2 siRNA Transfections and Overexpression

Gastric cancer cells were transfected with either specific siRNAs targeted to the CD44-SLC1A2 fusion site (100 nM, custom siRNA siGENOME with SMART selection, Dharmacon) or negative control scrambled siRNAs using siPORT™ NeoFX™ transfection reagent (Applied Biosystem) in Optimem Medium (Invitrogen) following the manufacturer's protocol. After 24, 48, and 72 hours siRNA treatment, cells were subjected for downstream analysis. For overexpression studies, the full length coding regions of CD44-SLC1A2 cDNA were inserted into the pEGFP-N1 vector. Control vectors or fusion-GFP vectors were introduced into HFE145 cells, and stable transfectants were selected using puromycin for 4 weeks.

Cell Proliferation Assay and Invasion Assays

Cell proliferation assays were performed using a CellTiter-96 Aqueous Nonradioactive Cell Proliferation Assay kit (Promega) following the manufacturer's instructions and the plates were measured by PerkinElmer plate reader. Cell invasion assays were performed using Biocoat™ Matrigel™ invasion chambers with 8 μm pore filter inserts (BD Bioscience). 48 hours after transfection, 5×104 cells were transferred to the upper matrigel chamber in 500 μl of serum-free medium and incubated for 24 hours. Invading cells were counted using light microscopy. Each assay was performed in triplicate, and the results were averaged over three independent experiments.

Colony Formation Assays

Base layers of 0.5% Gum Agar in 1× McCoy's 5 A and 10% FBS were poured into 6-well plates and allowed to harden at 4° C. After 48 hours siRNA transfection, 50,000 cells/well were seeded in complete media plus agar mixture at 42° C. and seeded on top of the solidified base layer. Plates were incubated at 37° C. in for 3-4 weeks, during which plates were fed drop-wise with complete media. After 3-4 weeks, plates were photographed using the Kodak GL 200 System (EpiWhite illumination). Each assay was performed in triplicate, and the results were averaged over three independent experiments.

Glutamate Assays and Drug Treatments

Gastric cancer cells and primary tissues were lysed with glutamate assay buffer and glutamate concentrations were determined using a Glutamate Assay Kit (BioVision, USA). Briefly, to each cellular lysate, a vendor-provided glutamate Enzyme Mix was added which recognizes glutamate as a specific substrate leading to proportional color development. For cisplatin treatments, cells were seeded into 96 well plates after siRNA transfection. Subsequently, cisplatin or 5-FU at increasing dosages were added (0-1 mM) to respective wells. Cells were subjected to MTS proliferation assays after 48 hours of drug treatment. Each assay was performed in triplicate, and the results were averaged over three independent experiments.

3′RACE

RLM-RACE was performed using the FirstChoice® RLM-RACE kit (Applied Biosystems). 1 μg of total RNA was reverse transcribed using 3′RACE adaptor and reverse transcriptase provided in the kit. To amplify first strand cDNAs, outer 3′ PCR was performed using 3′RACE outer primers and a SLC1A2 exon 1 primer (5′-TTGAGGCGCTAAAGGGCTTACC-3′ (SEQ ID NO: 10)) with SuperTaq™ Plus polymerase (Applied Biosystems). Subsequently, inner 3′ PCR was performed using a 3′ RACE inner primer (provided in kit) and a SLC1A2 exon 1 primer (5′-30 CAGACCATGGCATCTACGGAAGG-3′ (SEQ ID NO: 11)). After gel electrophoresis, PCR bands of interest were excised and cloned into pCR® 4-TOPO® (Invitrogen) vectors. Purified plasmid DNAs were sequenced bi-directionally on an ABI 3730 automated sequencer (AppliedBiosystems).

Immunofluorescence Staining

Cells were fixed with 3.7% formaldehyde followed by permeabilisation using 0.1% Triton X-100. After 3× washes with 1×PBS, cells were blocked with 1% BSA. Subsequently, cells were incubated with primary SLC1A2 antibodies (Cell Signaling Technology) for 2 hours followed by 2 hours secondary antibody (Sigma) incubation. Images were taken using a Nikon Eclipse TE2000-U microscope.

SLC1A2 Exon 1 siRNA Transfections

Gastric cancer cells were transfected with specific siRNAs targeted to SLC1A2 exon 1 (100 nM, custom siRNA siGENOME with SMART selection, Dharmacon) or negative control scrambled siRNAs using siPORT™ NeoFX™ transfection reagent (Applied biosystem) in Optimem Medium (Invitrogen) following the manufacturer's protocol.

Copy Number Analysis (Affymetrix)

Affymetrix SNP6 arrays were processed using Affymetrix GTC 4.0 software and tumor profiles were normalized against a matched normal reference. The data was visualized using Nexus 5.0 software (Biodiscovery). A Rank Segmentation algorithm, a variation of the segmentation method based on Circular Binary Segmentation (51) was used to segment the copy number data across the genome.

Gene Expression Analysis

Gene expression data is available from the Gene Expression Omnibus under access number GSE15460. Gene expression profiles (Affymetrix U133P2 arrays) were normalized using the MASS algorithm. Comparisons between CD44 and SLC1A2 expression values was performed on a subset of 45 samples for which gene expression, copy number information, and CD44-SLC1A2 gene fusion status was available. Unsupervised clustering was based on all probe sets after removing the bottom 25% of probes with the lowest interquantile range. Hierarchical clustering and Wilcoxson signed rank tests were performed using R software 2.9.0. FDR q value calculations were calculated using the R package ‘qvalue’. GO analysis was performed using the DAVID database (S2, S3). Gene expression data for survival analysis was obtained from a previously described cohort of 200 primary tumors profiled with Affymetrix Human Genome U133 plus Genechips (HG-U133 Plus 2.0, Affymetrix). Clinical data was obtained with National University of Singapore Institutional Review Board and available from 197 patients. Microarray data is available in the GEO database (GSE 15460).

QPCR

Gastric cancer cell lines and 9 primary gastric tumors were selected for QPCR. T1, T2, T3 were group 1 gastric tumors which are 11p13 amplification negative and fusion negative; T4, T5, T6 represent group 2 tumors which are 11p13 amplified but do not express CD44-SLC1A2; T7, T8, T9 were group 3 tumors expressing CD44-SLC1A2 but are non-11p13 amplified. Briefly, 2 μg RNA were reverse transcribed by SuperScript III reverse transcriptase enzyme using oligo-dT (T18) primers (Invitrogen). QPCR was performed using Quantifast SYBR green PCR kit (Qiagen) following manufacturer's instruction. Primers used were: GAPDH forward primer (5′-CCACCCAGAAGACTGTGGATGG-3′ (SEQ ID NO: 12)) and reverse primer (5′-CACTGACACGTTGGCAGTGG-3′ (SEQ ID NO: 13)); CD44 exon 2 forward primer (5′-TATAACCTGCCGCTTTGCAG-3′ (SEQ ID NO: 14)) and reverse primer (5′-GCAGGTCTCAAATCCGATGC-3′ (SEQ ID NO: 15)); SLC1A2 exon 1 forward primer (5′-GCCCGTTGAGGCGCTAAAGG-3′ (SEQ ID NO: 16)) and reverse primer (5′-AGCACTATCCGGCAGCTGTG-3′ (SEQ ID NO: 17)); Fusion forward primer targeting CD44 exon 1 (5′-TTCGGTCCGCCATCCTCGTC-3′ (SEQ ID NO: 18)) and reverse primer targeting SLC1A2 exon 2 (5′-CACTTCCACCTGCTTGGGCA-3′ (SEQ ID NO: 19)). Samples were run using a Applied Biosystem 7900HT system.

Statistical Analysis

Experiments were assessed by Student's unpaired t test, with the exception of the tumor/normal glutamate measurements where a paired t-test was used. P values <0.05 were considered statistically significant. Survival analysis was performed using SPSS software (version 16, Chicago). Overall survival curves were estimated using the Kaplan-Meier method. Death due to any cause was regarded as an event and patients still alive or lost to follow-up at time of analysis were censored at their last date of follow up. Survival analysis was performed with the Log Rank test, with 2 sided p-values of <0.05 being considered significant.

### Analysis of Copy Number Alterations Identifies Recurrent SLC1A2/EAAT2 Genomic Breakpoints in Gastric Cancer

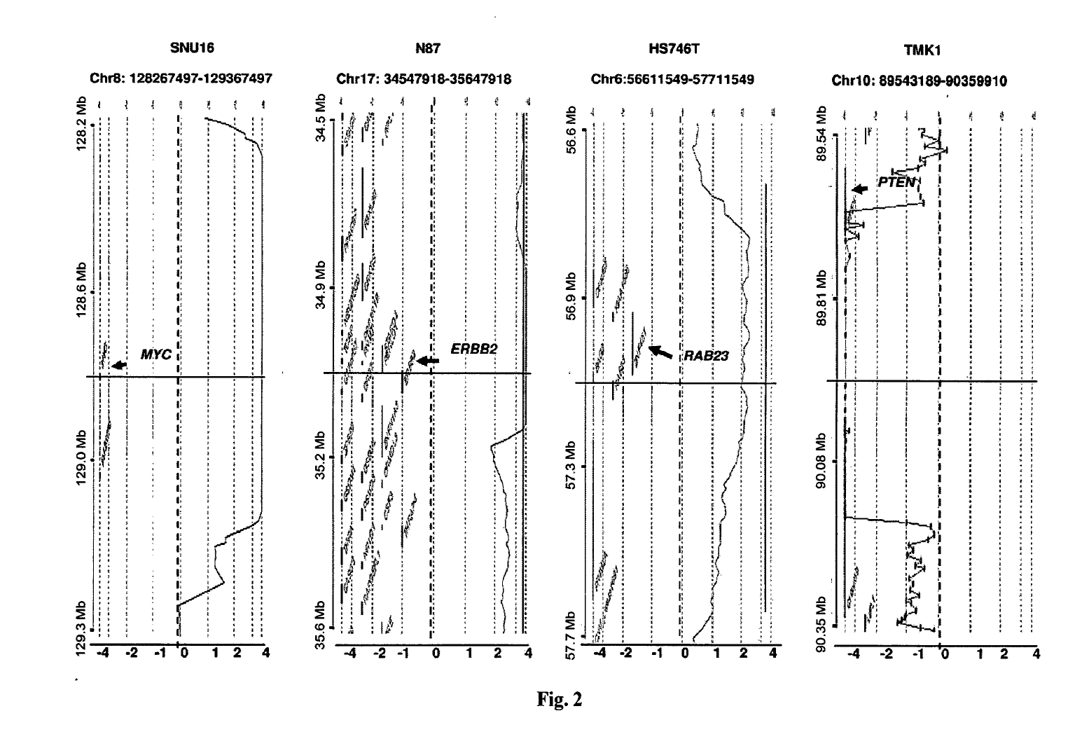

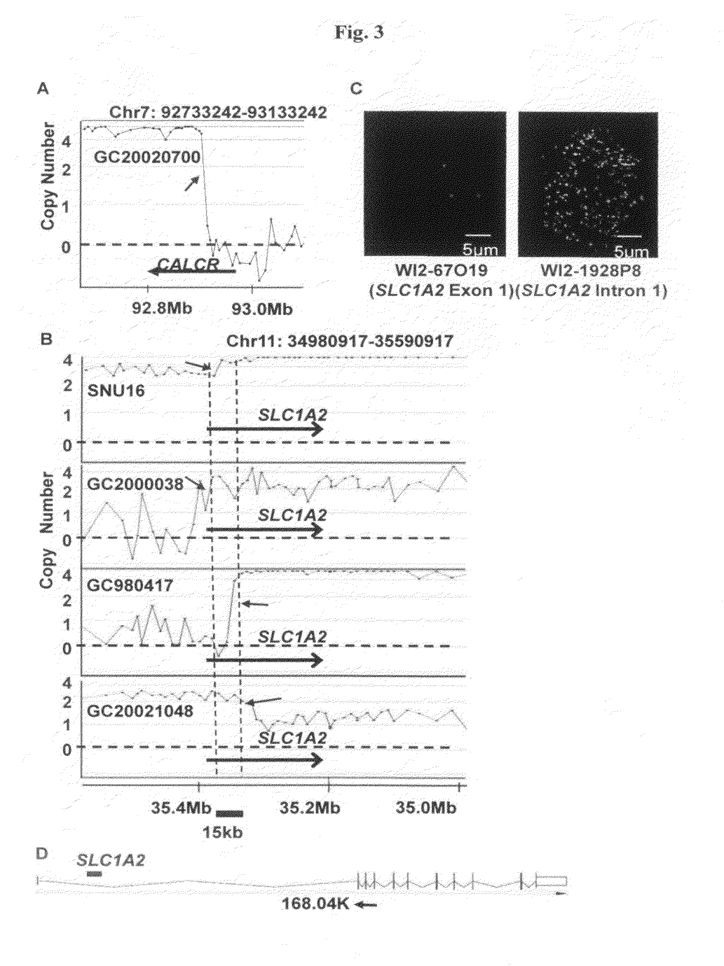

To locate genes which are disrupted by fusion events, a detailed fine-scale survey of genomic copy number alterations (CNAs) in gastric cancer was carried out. Using high density array-based comparative genomic hybridization (aCGH) microarrays, a discovery cohort of 133 gastric cancer samples (106 primary tumors and 27 cell lines) was profiled. Validating the array-based comparative genomic hybridization data, several previously identified genomic aberrations in gastric cancer was identified. The re-identified genomic aberrations include amplifications in c-Myc, HER2, RAB23, and PTEN deletions (FIG. 2).

To nominate potential fusion genes, a technique called Genomic Breakpoint Analysis (GBA), previously used to identify fusion genes in leukemia was employed. In this strategy, putative chromosomal breakpoints were identified by examining closely-spaced microarray probes displaying prominent transitions in copy number status, from low to high copy number or vice versa. FIG. 3A provides a representative example of a genomic breakpoint in the CALCR gene. In total, 99 genomic breakpoints occurring in genes such as CALCR, PERLD1, and CKAP5 were identified (Table 1).

TABLE 1
Gene Exhibiting Genomic Breakpoints

| | | | Tumor/ |
|---|---|---|---|
| Gene | Location | Genomic Event | Cell Line ID |
| CRKRS | 17q12 | Focal Amplification | 90929219, |
| | | | 2000484, |
| | | | 73499299, |
| | | | 2001140 |
| SLC1A2 | 11p13 | Focal Amplification | 980417, |
| | | | 20021048, |
| | | | 2000038, |
| | | | SNU16 |
| ZNFN1A3 | 17q21 | Focal Amplification | 20020164, |
| | | | 9874831, |
| | | | 54115380, |
| | | | YCC9, N87 |
| GSDML | 17q12 | Focal Amplification | 90929219, |
| | | | 54115380 |
| WIRE | 17q21.1-17q21.2 | Focal Amplification | 2000484, |
| | | | 2001140 |
| SMARCE1 | 17q21.3 | Focal Amplification | 2000484, |
| | | | 2001140 |
| CKAP5 | 11p11.2 | Focal Amplification | 2000763, |
| | | | 2000563 |
| PERLD1 | 17q12 | Focal Amplification | 20020164, |
| | | | 54115380 |
| LDLRAD3 | 11p13 | Focal Amplification | 20021048, |
| | | | 20020448 |
| TACC2 | 10q26 | Focal Amplification | 980417, |
| | | | 20020700 |
| KIAA0319 | 14q13.2 | Focal Amplification | YCC9, N87 |
| TTC25 | 17q21.2 | Focal Amplification | YCC9, N87 |
| CDH26 | 20q13.33 | Deletions | MKN74, MKN28 |
| TRERF1 | 6p21.1-p12.1 | Focal Amplification | YCC1, YCC6 |
| PEX7 | 6q23.3 | Amplification | 990020 |
| KIF6 | 6p21.2 | Amplification | 990428 |
| PDSS2 | 6q21 | Focal Amplification | 980447 |
| POPDC3 | 6q21 | Focal Amplification | 980447 |
| FLJ20294 | 11p11.12 | Focal Amplification | 980447 |
| ELF5 | 11p13-p12 | Focal Amplification | 2000038 |
| RASSF8 | 12p12.3 | Focal Amplification | 990428 |
| DYM | 18q12-21.1 | Focal Amplification | 990221 |
| LOC126248 | 19q13.11 | Focal Amplification | 980255 |
| CAT | 11p13 | Focal Amplification | 20021048 |
| c13orf24 | 13q22.1 | Amplification | 980021 |
| BCAT1 | 12p12.1 | Focal Amplification | 980024 |
| PDE1A | 2q32.1 | Amplification | 980024 |
| LST-3TM12 | 12p12.2 | Focal Amplification | 980024 |
| IDH1 | 2q33.3 | Amplification | 20020164 |
| ZNF403 | 17q12 | Focal Amplification | 20020164 |
| SOCS7 | 17q12 | Focal Amplification | 20020164 |
| RAB3IP | 12q14.3 | Amplification | 20020563 |
| RFFL | 17q12 | Focal Amplification | 20020700 |
| KLHL10 | 12q14.3 | Focal Amplification | 20020700 |
| CALCR | 17q12 | Focal Amplification | 20020700 |
| MULK | 17q21.2 | Amplification | 2001098 |
| DSCR3 | 21q22.2 | Focal Amplification | 2000710 |
| TP53I11 | 11p11.12 | Focal Amplification | 2000763 |
| FLJ46154 | 11p14.1 | Amplification | 2000763 |
| KIF14 | 1q32.1 | Amplification | 90929219 |
| TMEM116 | 12q24.13 | Focal Amplification | 90929219 |
| MAPKAPK5 | 12q24.12-q24.13 | Focal Amplification | 90929219 |
| C17ORF63 | 17q11.2 | Amplification | 9874831 |
| TNKS | 8p23.1 | Focal Amplification | 42761681 |
| FBXL20, | 17q12 | Focal Amplification | 54115380 |
| PPARBP | 17q12-q21.1 | Focal Amplification | 54115380 |
| TSHZ2 | 20q13.2 | Deletions | 73499299 |
| SLC24A3 | 20p13 | Amplification | 73499299 |
| SCARA5 | 8p21.1 | Amplification | 20020448 |
| RC74 | 8p21.1 | Amplification | 20020448 |
| MTSS1 | 8p22 | Focal Amplification | 20020448 |
| PROK2 | 3p13 | Deletions | 37175329 |
| ST7 | 7q31.1-q31.3 | Focal Amplification | 20020070 |
| WNT2 | 7q31.2 | Focal Amplification | 20020070 |
| STARD3 | 17q11-q12 | Focal Amplification | 2001140 |
| CCR7 | 17q12-21.2 | Focal Amplification | 2001140 |
| PREP | 6q16.3-q22.1 | Focal Amplification | 980447 |
| RPA2 | 1p35.1 | Amplification | YCC9 |
| RPAIN | 17p13.2 | Amplification | FU97 |
| SYN | 22q12.3 | Deletions | SCH |
| LRP1B | 2q21.2 | Focal deletions | SCH |
| DGKG | 3q27.3 | Deletions | SCH |
| PDE4D | 5q12 | Focal deletions | SCH |
| CCDC91 | 12p11.22 | Deletions | MKN74 |
| COL24A1 | 1q22.3 | Amplification | MKN28 |
| PAX5 | 9p13 | Amplification | MKN28 |
| MYO18A | 17q11.2 | Amplification | MKN1 |
| STK38L | 12p11.23 | Focal Amplification | MKN1 |
| SORT1 | 1p21.3-p13.1 | Focal Amplification | MKN45 |
| CADPS2 | 7q31.3 | Focal Amplification | MKN45 |
| MAP2K4 | 17p11.2 | Deletions | MKN45 |
| KIF18A | 11p14.1 | Focal Amplification | MKN45 |
| TBC1D22A | 22q13.3 | Deletions | MKN45 |
| PEPD | 19q12-q13.2 | Focal Amplification | TMK1 |
| PTEN | 10q23.3 | Focal deletions | TMK1 |
| LSM14A | 19q13.11 | Focal Amplification | TMK1 |
| ATF7IP2 | 16p13.13 | Amplification | YCC7 |
| DNM2 | 19p13.2 | Amplification | YCC7 |
| WDR40A | 9p13.3 | Amplification | YCC7 |
| ZCCHC7 | 9p13.2 | Amplification | YCC7 |
| MN1 | 22q12.1 | Amplification | YCC7 |
| FLJ33814 | 22q12.1 | Amplification | YCC7 |
| EMID1 | 22q12.2 | Amplification | YCC7 |
| NF2 | 22q12.2 | Amplification | YCC7 |
| RBM9 | 22q13.1 | Amplification | YCC7 |
| CPE | 4q32.3 | Deletions | SNU1 |
| LRBA | 4q31.3 | Deletions | SNU5 |
| ATE1 | 10q26.13 | Focal Amplification | SNU16 |
| CLCA4 | 1p31-p22 | Deletions | YCC2 |
| MEP1A | 6p12-p11 | Focal Amplification | YCC2 |

For the majority of genes exhibiting genomic breakpoints in multiple samples (e.g. CRKRS, TTC25), the breakpoints were randomly scattered throughout the gene body consistent with a random breakage model of chromosomal amplification. However, four gastric cancer samples out of 133 (three primary tumors and one cell line—GC980417, GC20021048, GC2000038 and SNU16) exhibited genomic breakpoints specifically localized to the 5′ region of the SLC1A21EAAT2 gene, which encodes a high affinity glutamate transporter (henceafter referred to as SLC1A2) (FIG. 3B). To validate the SLC1A2 breakpoint region, fluorescence in-situ hybridization (FISH) analysis using fosmid probes mapping upstream or downstream to the putative breakpoint (WI2-67019 and WI2-1928P9) was performed. Supporting the aCGH data, the WI2-67019 upstream probe (35384118-35427600) covering the first exon of SLC1A2 showed 3-4 signals in SNU16 nuclei (FIG. 3C, left), confirming previous studies that SNU16 is a naturally tetraploid cell line. In contrast, the downstream WI2-1928P9 probe (35323126-35359663) located at SLC1A2 intron 1 showed multiple hybridization signals (>50 copies) indicating a high level amplification event (FIG. 3C, right).

Accordingly, the inventors found the existence of CD44-SLC1A2 gene fusions in gastric cancer. Notably, while genomic breakpoint analysis has been previously used for fusion gene discovery in leukemia, it was demonstrated that this approach can also highlight potential fusion genes in solid epithelial tumors. Amongst genes exhibiting genomic breakpoints, priorities on the genes studied were chosen based on i) their rate of recurrence in multiple samples, and ii) occurrence in a cell line to serve as an experimental model. Using these two criteria, only two genes were nominated—SLC1A2 and ZNF1A3. It was noted that breakpoint analysis does come with a few caveats, as fusion events arising from balanced chromosomal rearrangements are unlikely to be detected, and would not alter overall copy number levels. However, genomic breakpoint analysis has the advantage of being readily applicable to array-CGH data, for which there are already numerous large scale data sets readily available in the public domain. Revisiting these data sets may identify additional genes recurrently targeted by genomic breakpoints in solid cancers.

The identification of SLC1A2, a glutamate transporter, as a fusion gene participant is notable for several reasons. To date, the vast majority of known oncogenic fusion events have tended to involve transcription factors (e.g. Myc, RAR, etc) or signal transduction proteins such as kinases (e.g. BCR-ABL). The discovery of CD44-SLC1A2 raises the intriguing possibility that oncogenic gene fusions may also target genes involved in cancer metabolism. Specifically, the CD44-SLC1A2 gene fusion is predicted to produce a slightly truncated SLC1A2 protein that retains most of the key protein domains required for glutamate transporter fusion, and may function to facilitate glutamate accumulation in gastric cancer cells. Indeed, a substantial body of evidence has implicated glutamate and glutamine as a critical amino acid necessary for the maintenance and elaboration of many cancer-specific traits. For example, glutamate and glutamine have been shown to regulate tumor growth and oncogenic signals such as mTOR. The requirement of cancer cells for glutamate may also be related to the Warburg effect—a universal feature of cancer cells where they exhibit overactive glycolysis due to a deficiency in channeling glycolytic metabolites into the TCA cycle for ATP generation. Besides glycolysis, glutamate may provide cancer cells with an alternative route of ATP production since intracellular glutamate and glutamine can also be converted into alpha-ketoglutarate, a TCA cycle intermediate. Glutamate levels have been shown to be elevated in many cancers, and in the present study found that glutamate levels are also elevated in gastric tumors compared to normal stomach.

### SLC1A2 Breakpoint Characterization Reveals a CD44-SLC1A2 Gene Fusion

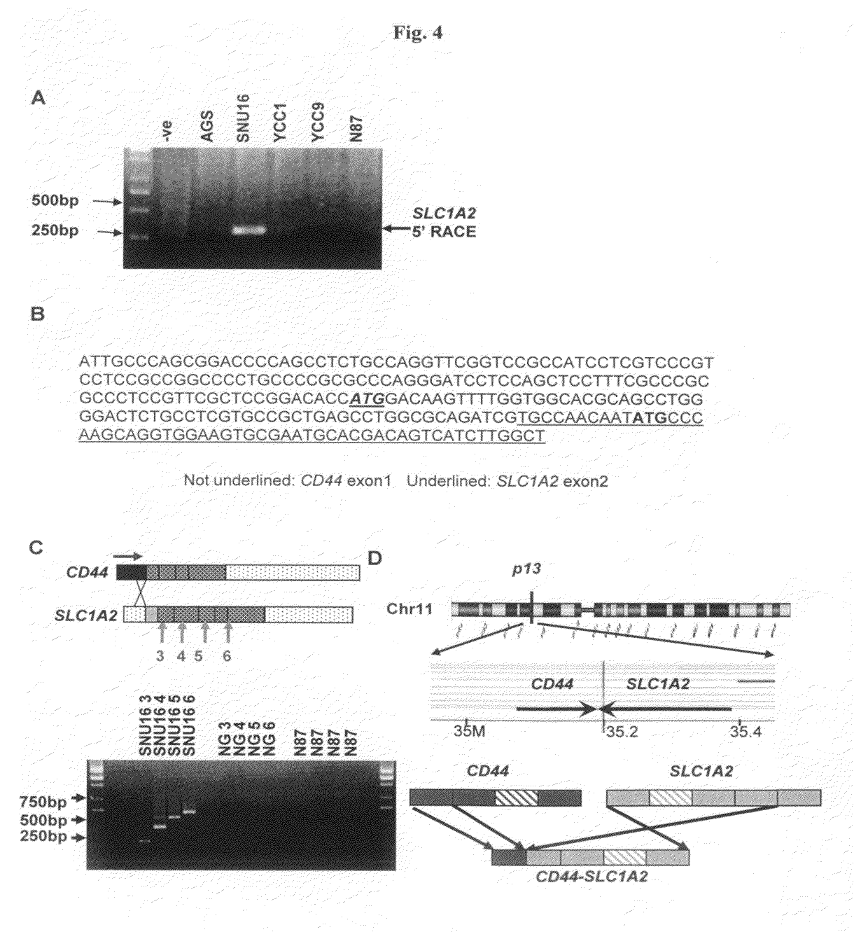

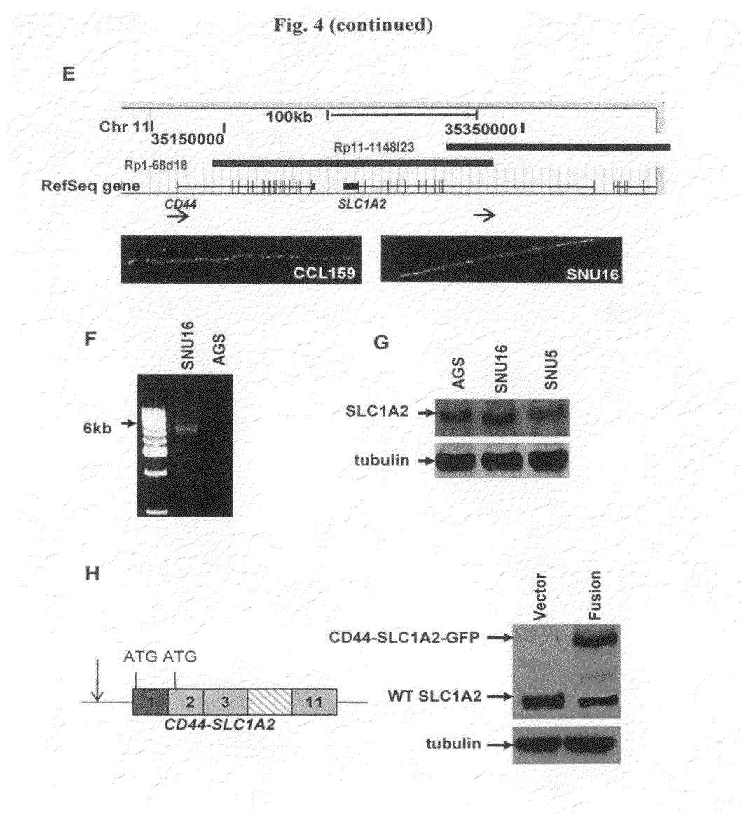

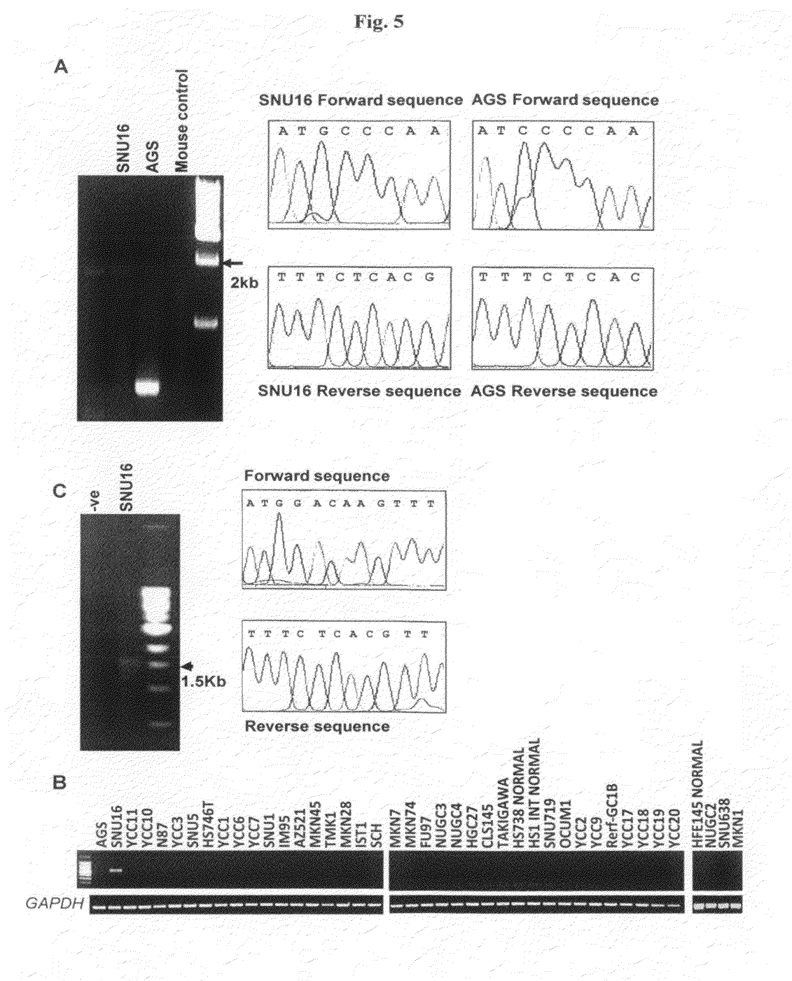

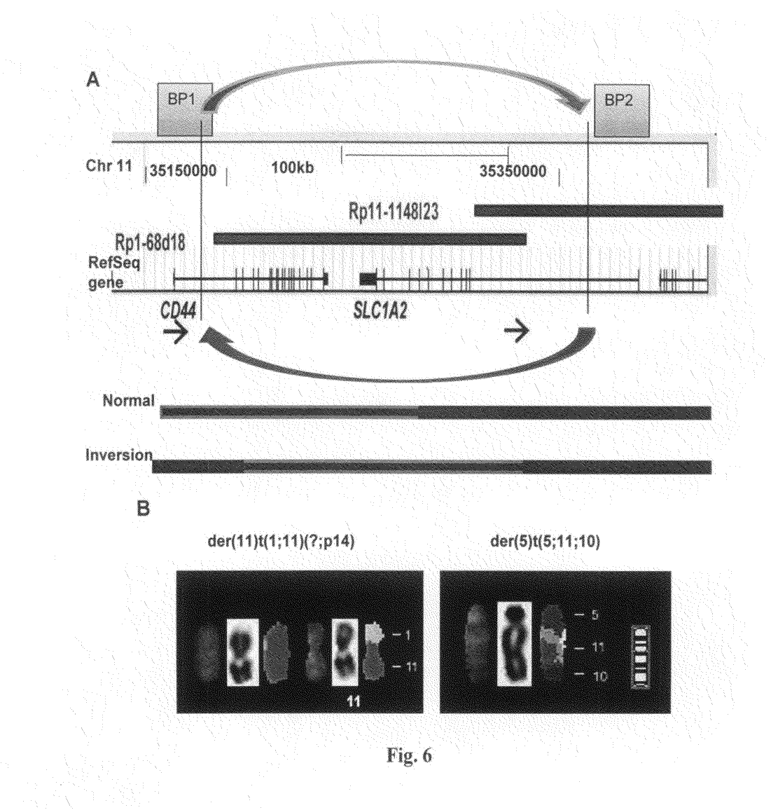

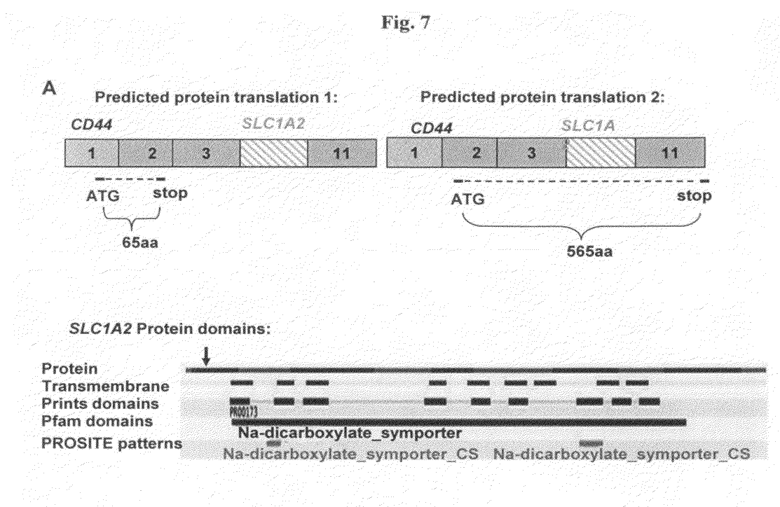

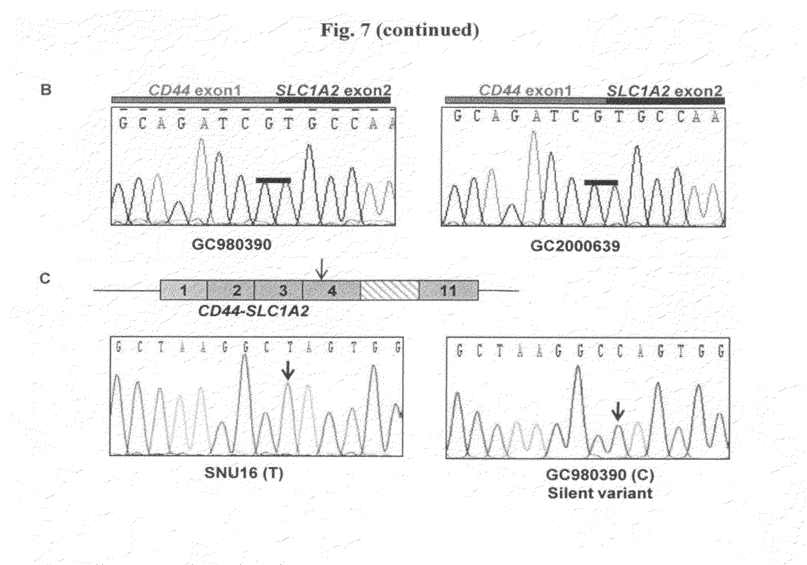

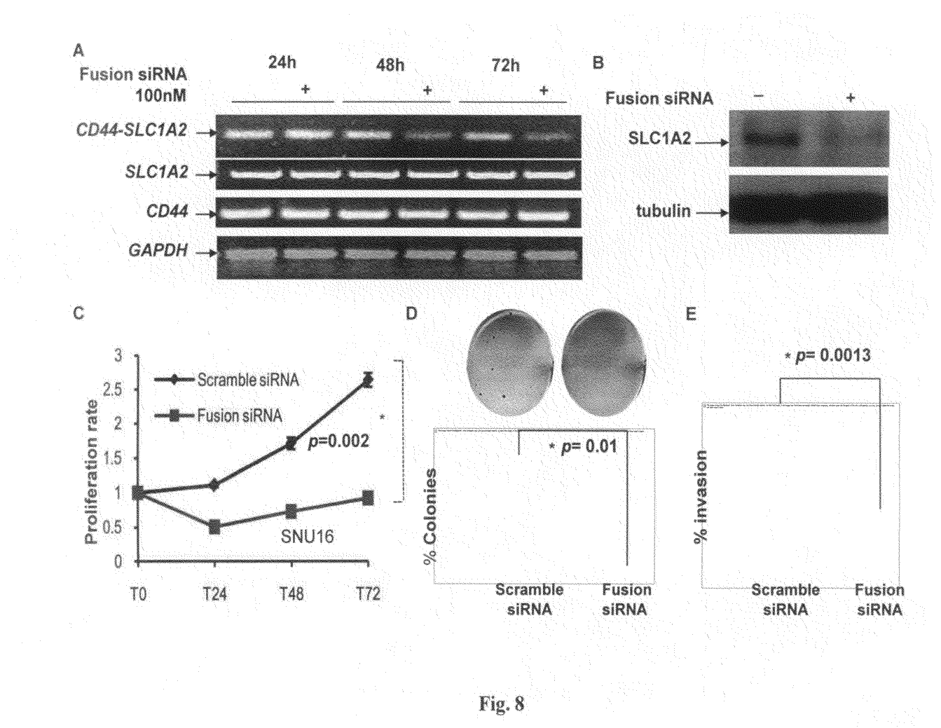

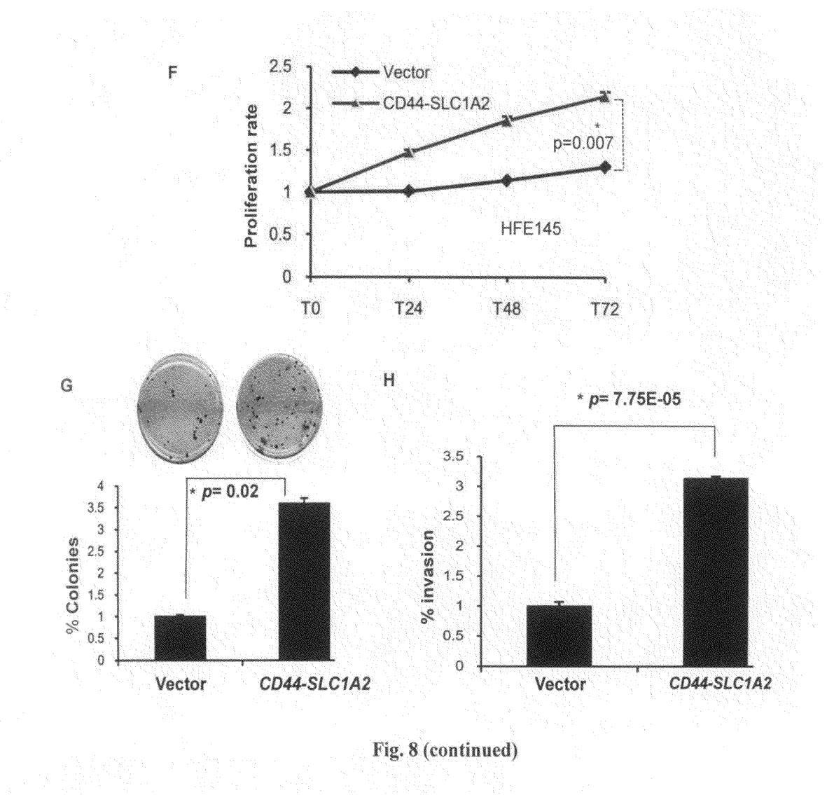

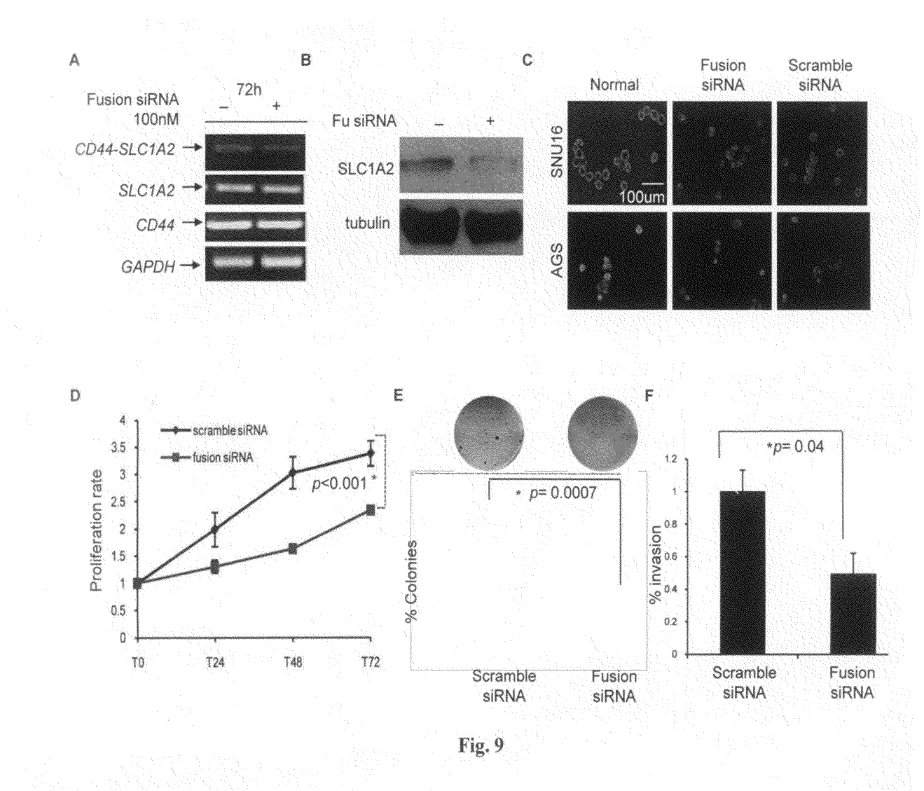

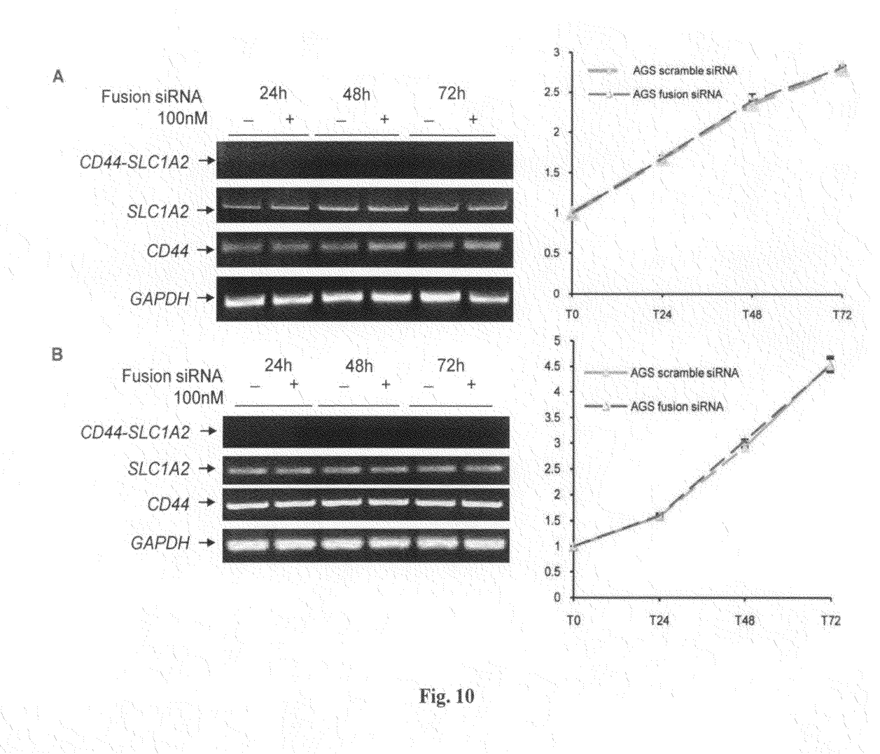

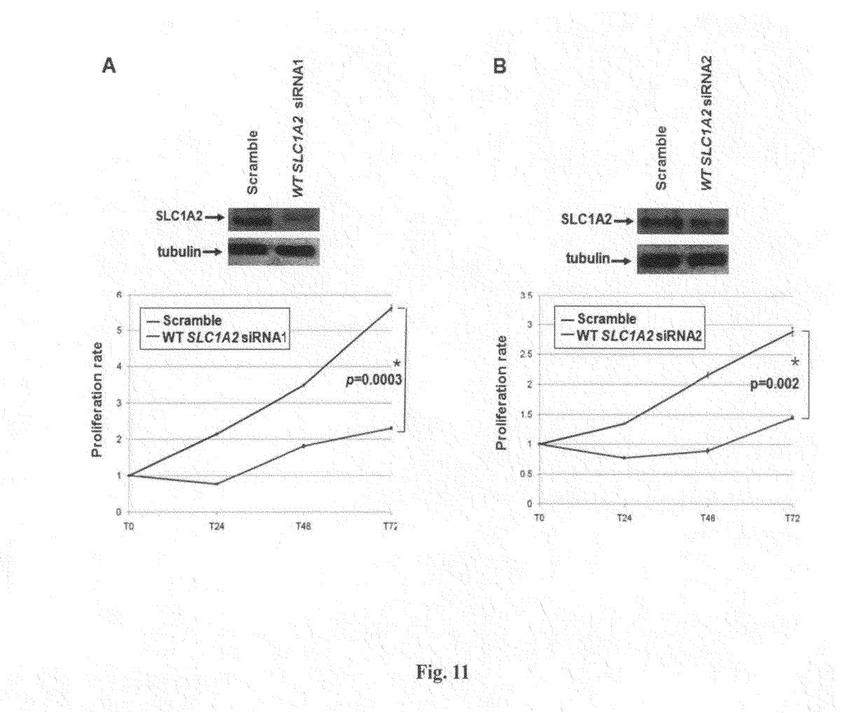

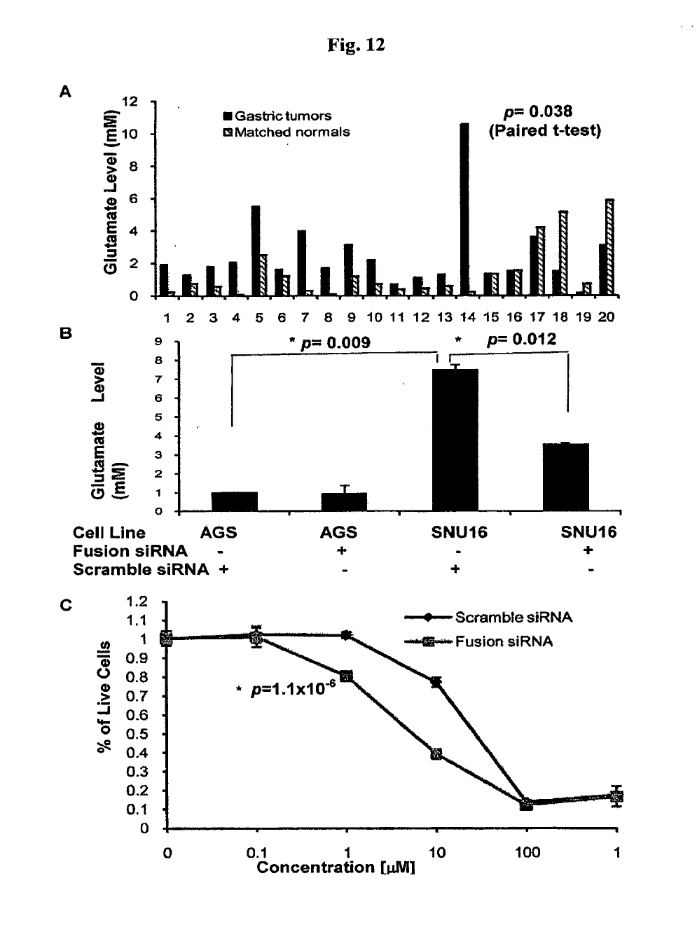

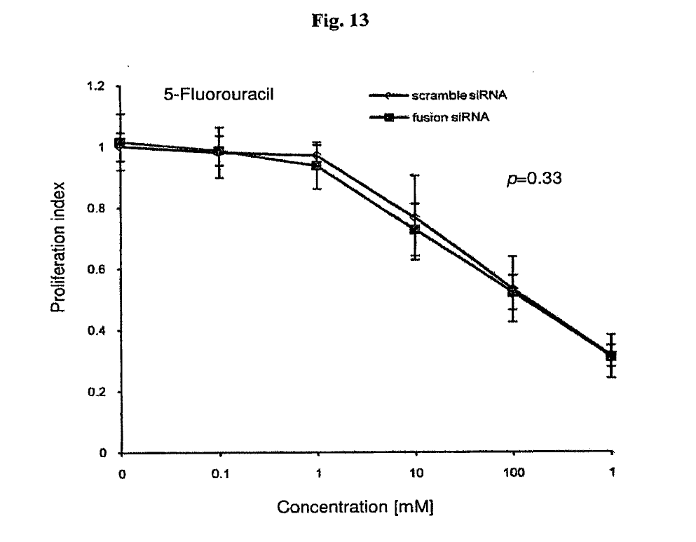

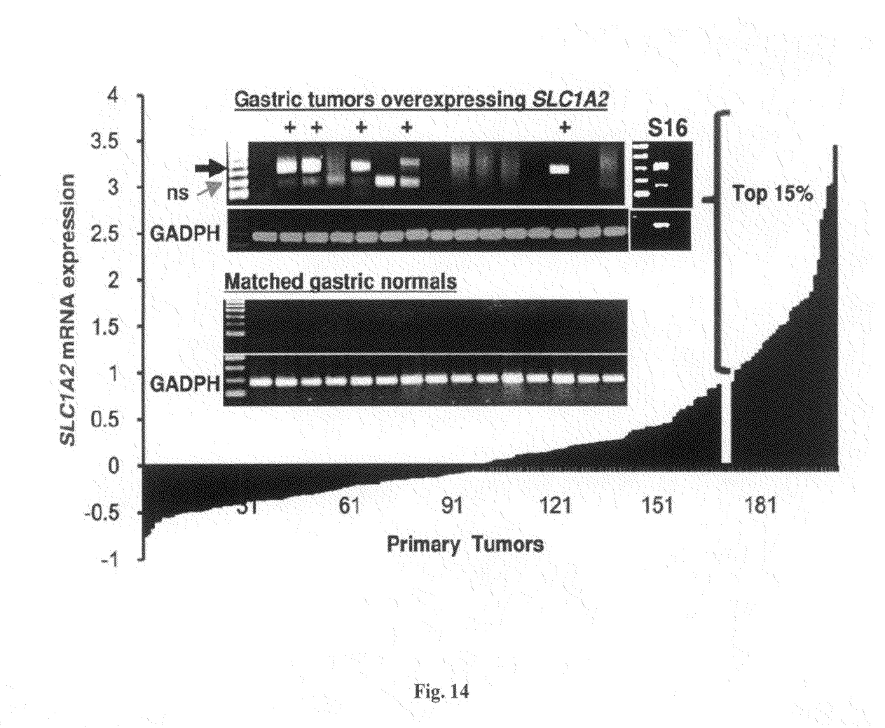

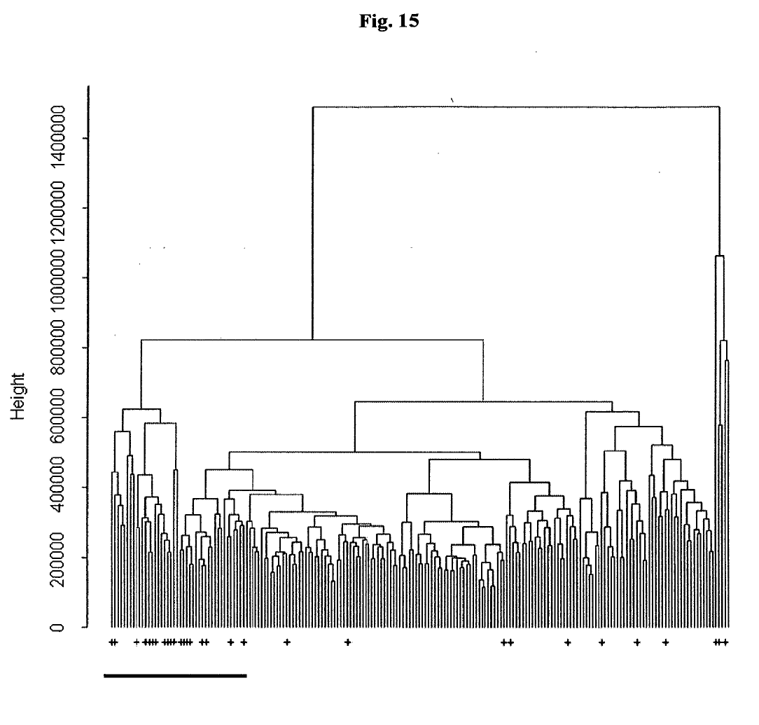

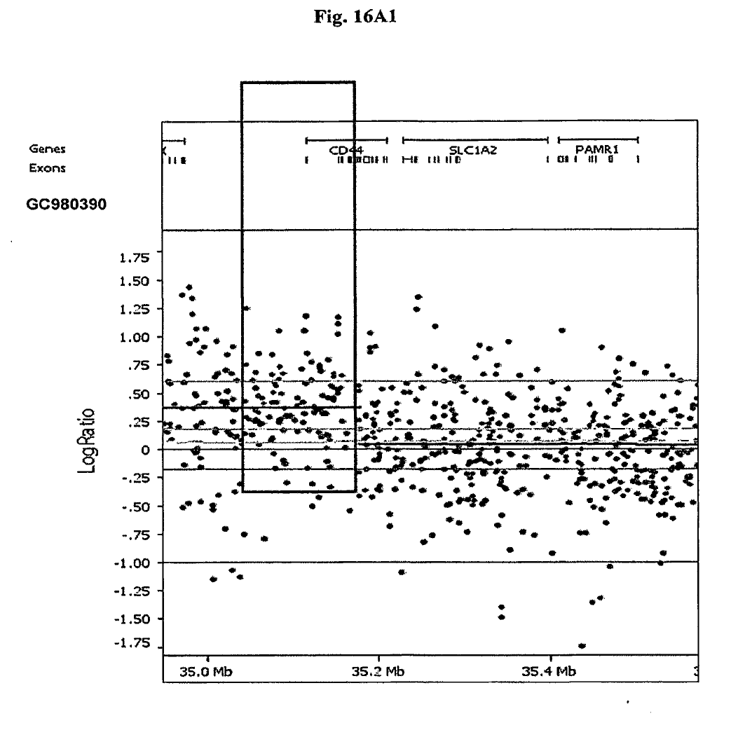

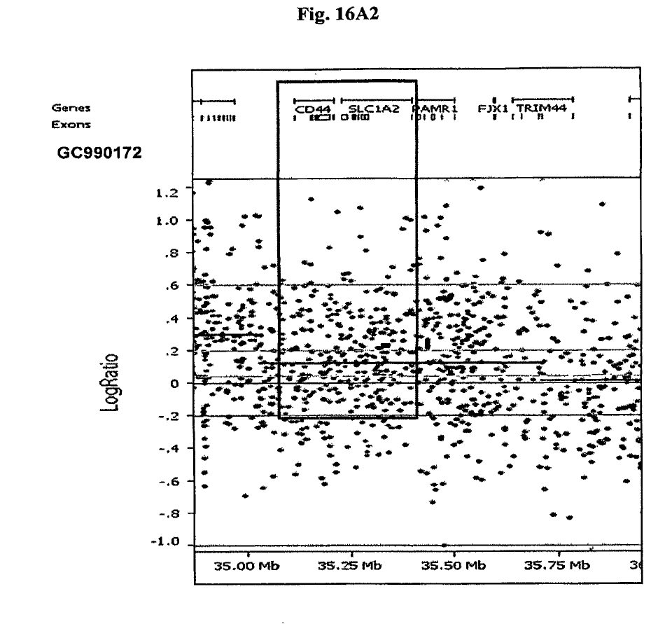

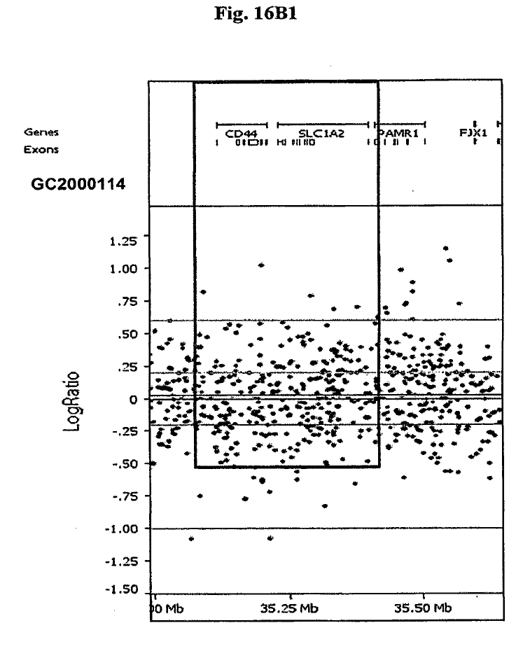

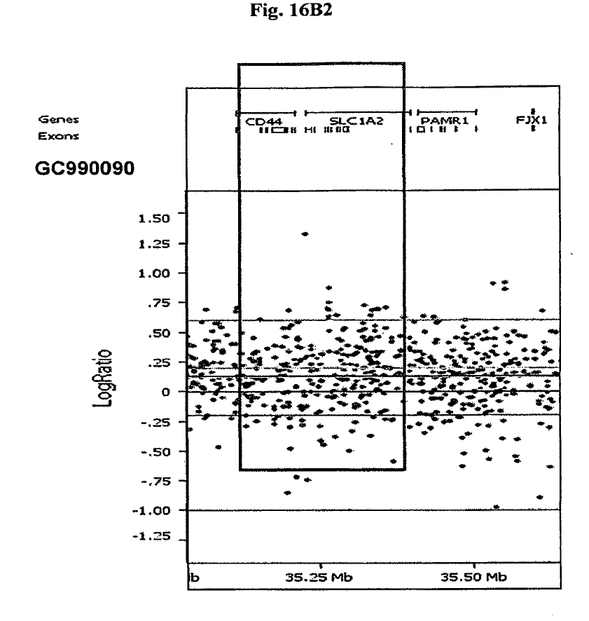

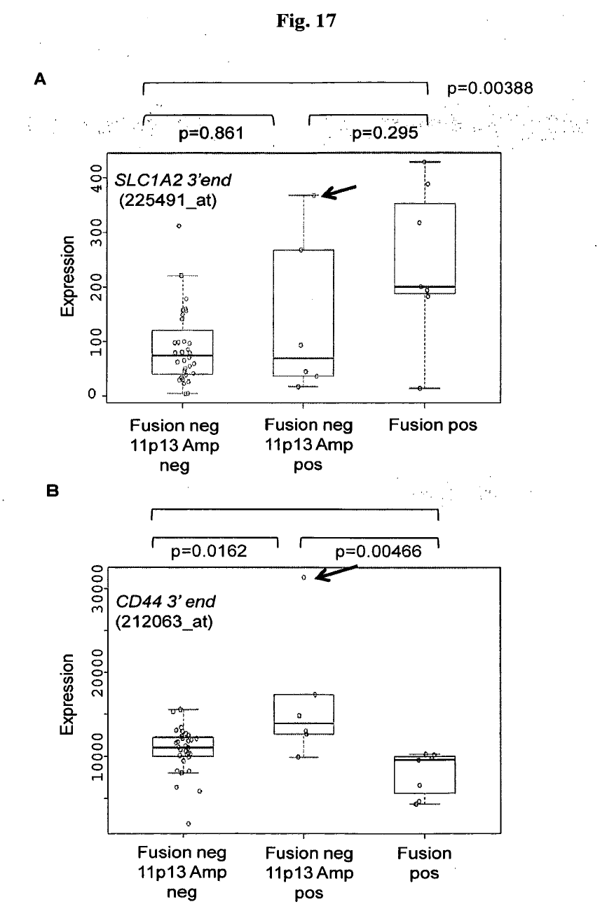

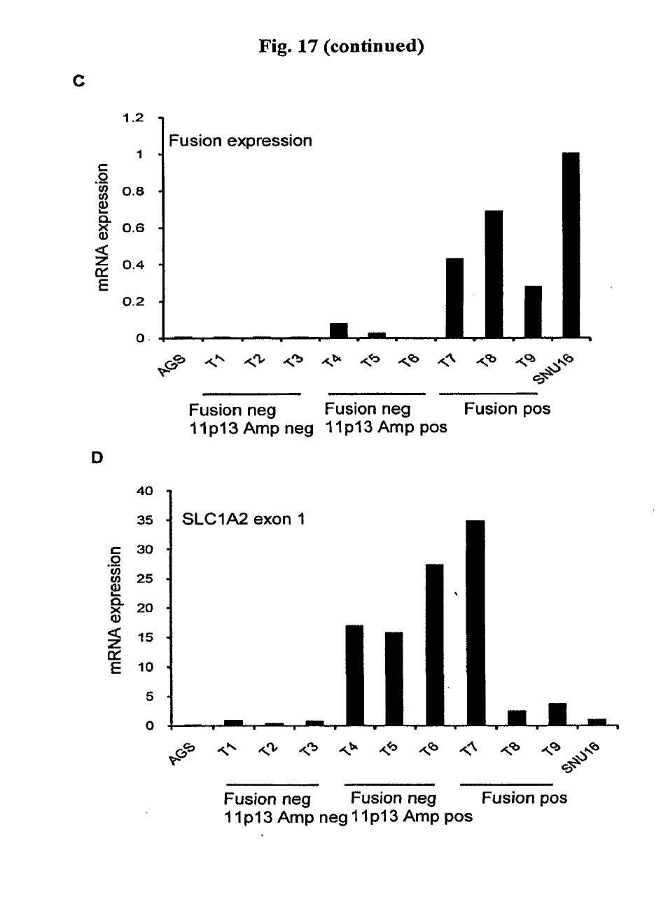

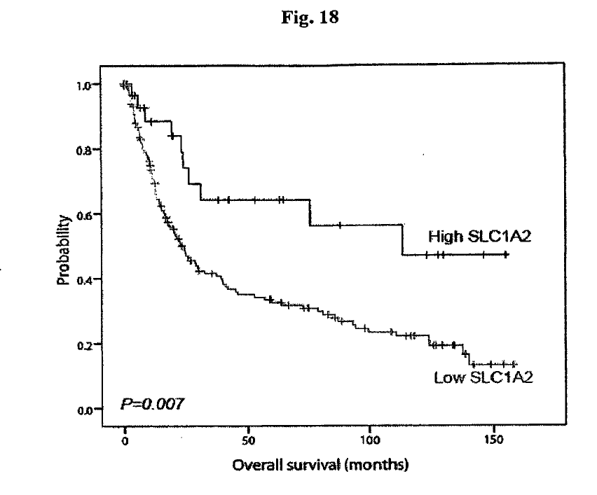

The SLC1A2 breakpoint regions from the aCGH and the FISH data was integrated to define a 15-24 kb minimal common breakpoint window in the SLC1A2 first intron (FIGS. 3B and 3D, black bar). The inventors hypothesized that chromosomal aberrations affecting this region might disrupt the SLC1A2 gene resulting in potential fusion partners. To test this, 5′ RNA Ligase-Mediated Rapid Amplification of cDNA Ends (RLM-RACE) was performed to characterize SLC1A2 transcript sequences upstream to SLC1A2 exon 2. A 5′ RACE product of 250 by was identified in breakpoint positive SNU16 cells, but not in other gastric cancer cell lines without SLC1A2 breakpoints (AGS, YCC1, YCC9 and N87) (FIG. 4A). Sequencing of the amplified SNU16 product revealed a CD44-SLC1A2 fusion transcript, formed by the juxtaposition of CD44 exon 1 to SLC1A2 exon 2 (FIG. 4B). In complement to the 5′ RACE analysis, 3′ RACE was also performed in SNU16 cells analyzing transcripts downstream of SLC1A2 exon 1 and did not identify any additional fusion partners besides wild-type SLC1A2 transcripts (FIG. 5A). To validate the 5′ RACE results, combination sets of PCR primers targeting CD44 exon 1 (forward primer; SEQ ID NO:3) and SLC1A2 exons 3, 4, 5, 6 (reverse primers; SEQ ID NO: 4, 5, 6, 7, respectively) to directly detect the fusion by reverse transcription PCR (RT-PCR) was designed. CD44-SLC1A2 transcripts were detected in SNU16 cells, but not in other cell lines nor in commercially available normal gastric tissue (NG) (FIG. 4C and FIG. 5B). The expression of a complete ˜1.6 kb CD44-SLC1A2 transcript in SNU16 cells was confirmed using RT-PCR primers targeting CD44 exon 1 and SLC1A2 exon 11 (the last SLC1A2 exon) (FIG. 5C). These results demonstrated the existence of a CD44-SLC1A2 gene fusion in SNU16 cells.

The genes CD44 and SLC1A2 lie adjacent to each other on chromosome 11p13, being separated by only ˜19 kb (FIG. 4D). The two genes are transcribed towards each other, indicating that they possess distinct promoters. Because they lie on opposite strands, it is unlikely that the CD44-SLC1A2 fusion is caused by a transcriptional readthrough event. The inventors thus hypothesized that the CD44-SLC1A2 gene fusion might have been caused by a paracentric chromosomal inversion event (FIG. 5A). Spectral karyotyping (SKY) analysis of SNU16 cells confirmed the presence of at least two 11p13-11p14 genome rearrangements in this region, one involving fusion of Chromosome 1 with Chromosome 11 at band 11p13-14, and the second involving a complex chromosomal scenario with rearrangements joining chromosomes 5, 10 and 11 (FIG. 5B).

To verify the presence of CD44/SLC1A2 genomic inversions in this region, two different strategies were employed. First, fiber-FISH, a high-resolution method for genomic DNA mapping was used. Fosmid probes Rp1-68d18 (35146316-35329998, covering the CD44 gene and a portion of the SLC1A2 gene) and Rp11-1148123 (35294107-35461767, covering the SLC1A2 gene only) were hybridized to SNU16 cells or normal lymphoblastoid CCL159 cells. In control CCL159 cells, a normal chromosome indicated by two distinct red and green probe signals lying adjacent to one another was observed. In contrast, in SNU16 cells a “split-apart” red-green-red signal, consistent with an inversion event occurring between these probes (FIG. 4E) was observed. Second, the presence of a CD44/SLC1A2 genomic inversion in SNU16 cells was directly confirmed using long-range genomic PCR, followed by end-sequencing of the PCR products. Using primers located to CD44 exon 1 and the SLC1A2 1st intron (black arrows in FIG. 4E), CD44/SLC1A2 inversion product in SNU16 fusion-positive cells but not AGS cells was successfully PCR amplified and its sequence validated (FIG. 4F). Taken collectively, these two alternative methods confirmed the presence of a chromosomal inversion event in SNU16 occurring between CD44 and SLC1A2.

Sequence analysis of the CD44-SLC1A2 fusion revealed two distinct protein translation patterns (FIG. 7A). First, translation initiating from an ATG site in the CD44 exon one could produce a 65 amino acids protein, comprising 22 amino acids of CD44 and 43 amino acids of novel sequence (SEQ ID NO: 20). Second, functional protein translation might also initiate from an alternative ATG site in SLC1A2 exon 2, downstream of the fusion site. Translation from this alternative ATG would produce a 565 amino acids truncated SLC1A2 protein that is 17 amino acids shorter than the full length form (SEQ ID NO: 21), but retaining all functionally relevant domains including transmembrane helices and symporter domains.

To test if CD44-SLC1A2 might produce a truncated SLC1A2 protein, Western Blotting using anti-SLC1A2 antibodies on fusion positive and fusion negative gastric cancer cells was performed. In SNU16 fusion-positive cells, a smaller-sized SLC1A2 protein compared to fusion negative AGS and SNU5 cells (FIG. 4G) was detected. This is consistent with translation initiating from SLC1A2 exon 2 rather than exon 1 in SNU16 cells. To further demonstrate that the alternative ATG in SLC1A2 exon 2 is capable of initiating protein translation, the full-length CD44-SLC1A2 fusion gene in HFE145 normal gastric cells was then cloned and expressed. Western blotting analysis confirmed expression of an immunoreactive SLC1A2 product in CD44-SLC1A2 transfected HFE145 cells of the expected size (FIG. 4H). This result demonstrated that the alternative ATG in SLC1A2 exon 2 is sufficient to initiate translation.

### Recurrent Expression of the CD44-SLC1A2 Gene Fusion in Primary Gastric Cancer Cells

To test if CD44-SLC1A2 might be expressed in clinical specimens, CD44-SLC1A2 expression in two of the three original index cases exhibiting SLC1A2 genomic breakpoints was screened (FIG. 3B). The third index tumor, GC20021048, had insufficient material available for analysis. CD44-SLC1A2 expression was detected in tumor GC2000038, but not in corresponding matched normal tissue (FIG. 1A). This result demonstrated CD44-SLC1A2 expression can occur in primary tumors and that it is not a “private” event confined to SNU16 cells alone.

CD44-SLC1A2 RT-PCR screening in an independent panel of forty-three gastric tumors and matched gastric normal tissues was also performed. Two additional tumors were identified expressing the CD44-SLC1A2 fusion transcript (FIG. 1B). Similar to the index samples, CD44-SLC1A2 was not expressed in corresponding matched normal samples (FIG. 1B, bottom), supporting the cancer specific nature of the fusion transcript. Subsequent cloning and sequencing of CD44-SLC1A2 in the fusion-positive tumors revealed that the fusion consistently involved the juxtaposition of CD44 exon 1 to SLC1A2 exon 2 (FIG. 1C and FIG. 7B). This apparent requirement for precise joining may be because amongst the SLC1A2 exons, only exon 2 possesses a suitable alternative start ATG to initiate translation of a near complete SLC1A2 protein. Using long-range PCR, the presence of CD44/SLC1A2 genomic inversions at the DNA level in two fusion-positive clinical specimens (GC980390 and GC2000038) was also confirmed (FIG. 1D). Importantly, no genomic inversion PCR products were observed in the matched normal gastric samples, indicating that the CD44/SLC1A2 inversion is likely a cancer-associated somatic event.

### CD44-SLC1A2 Silencing Reduces Cancer Cell Proliferation, Invasion, and Colony Formation

To investigate the functional consequences of inhibiting CD44-SLC1A2 expression, a series of customized siRNAs targeting the CD44-SLC1A2 fusion site was designed and provided as follows: 5′-CGCAGAUCGUGCCAACAAUUU-3′ (SEQ ID NO: 27; FIG. 8) and 5′-GCACAUCGUGCCAACAAUAUU-3′ (SEQ ID NO: 28; FIG. 9). Treatment of SNU16 cells with fusion specific siRNAs successfully silenced CD44-SLC1A2 expression, but did not discernibly alter the independent expression of CD44 or SLC1A2 (FIG. 8A). Silencing at the protein level was confirmed by both Western blotting and immunofluorescence assays using SLC1A2 antibodies (FIG. 8B and FIG. 9). Similar results were obtained using a second CD44-SLC1A2 targeting siRNA containing overlapping but distinct sequence (FIG. 9). These results confirmed the efficacy of the fusion-specific siRNAs.

SNU16 cells stably silenced with CD44-SLC1A2 siRNAs resulted in a significant reduction in cell proliferation capacity compared to cells treated with scrambled siRNAs (FIG. 8C, p=0.002, t-test). No effects were observed when the fusion specific siRNA was applied to AGS cells which do not express CD44-SLC1A2 (FIG. 10A-B). These results suggest that CD44-SLC1A2 may be important for cancer cell proliferation in gastric cancer. To assess the tumorigenicity of SNU16 upon CD44-SLC1A2 knockdown, colony formation assays were performed. Fusion-silenced cells exhibited a significantly decreased level of anchorage-independent growth compared to controls (p=0.01, FIG. 8D). Matrigel assays to investigate effects of CD44-SLC1A2 on cancer cell invasion was also performed. CD44-SLC1A2-silenced SNU16 cells also exhibited a decreased level of cell invasion compared with control cells (FIG. 8E, p=0.0013), suggesting a potential role for CD44-SLC1A2 in cell motility and invasion.

To ask if CD44-SLC1A2 expression might be sufficient to enhance various prooncogenic traits, CD44-SLC1A2 was stably overexpressed in HFE145 normal gastric cells. Compared to control cells, CD44-SLC1A2 expressing HFE145 cells exhibited enhanced cell proliferation (p=0.007), colony formation (p=0.02), and invasion (p=7.75×10-5) (FIG. 8F-H). Taken collectively, these results suggest that CD44-SLC1A2 is likely required by gastric cancer cells to maintain several pro-oncogenic traits, such as proliferation, colony formation and invasion.

The observation that CD44-SLC1A2 produces an almost full-length SLC1A2 protein lacking only 17 amino acids (SEQ ID NO: 21) raises the possibility that wild-type SLC1A2 might also be pro-oncogenic. Indeed, silencing of wild-type SLC1A2 in AGS cells, which are fusion negative, resulted in similar phenotypic effects comparable to CD44-SLC1A2 silencing in SNU16 cells (FIG. 11). In this regard, CD44-SLC1A2 may be similar to oncogenic fusion genes such as IgH-Myc and TMPPRS2-ERG, where an essentially full-length pro-oncogenic protein is placed under the control of a strong transcriptional promoter.

Sequence of CD44-SLC1A2 protein translation initiated from ATG site in SLC1A2 exon 2 is provided as follows:

(SEQ ID NO: 21)
Met P K Q V E V R Met H D S H L G S E E P K H R H L G L R L C D K L
G K N L L L T L T V F G V I L G A V C G G L L R L A S P I H P D V V
Met L I A F P G D I L Met R Met L K Met L I L P L I I S S L I T G L
S G L D A K A S G R L G T R A Met V Y Y Met S T T I I A A V L G V I
L V L A I H P G N P K L K K Q L G P G K K N D E V S S L D A F L D L
I R N L F P E N L V Q A C F Q Q I Q T V T K K V L V A P P P D E E A
N A T S A V V S L L N E T V T E V P E E T K Met V I K K G L E F K D
G Met N V L G L I G F F I A F G I A Met G K Met G D Q A K L Met V D
F F N I L N E I V Met K L V I Met I Met W Y S P L G I A C L I C G K
I I A I K D L E V V A R Q L G Met Y Met V T V I I G L I I H G G I F
L P L I Y F V V T R K N P F S F F A G I F Q A W I T A L G T A S S A
G T L P V T F R C L E E N L G I D K R V T R F V L P V G A T I N Met
D G T A L Y E A V A A I F I A Q Met N G V V L D G G Q I V T V S L T
A T L A S V G A A S I P S A G L V T Met L L I L T A V G L P T E D I
S L L V A V D W L L D R Met R T S V N V V G D S F G A G I V Y H L S
K S E L D T I D S Q H R V H E D I E Met T K T Q S I Y D D Met K N H
R E S N S N Q C V Y A A H N S V I V D E C K V T L A A N G K S A D C
S V E E E P W K R E K Stop.

### CD44-SLC1A2 Silencing Significantly Reduces Intracellular Glutamate Levels and Sensitizes Gastric Cancer Cells to Chemotherapy

One possible mechanism by which CD44-SLC1A2 may contribute to tumor development is by facilitating glutamate uptake in gastric cancer cells. In many cancers, glutamate and its related amino acid glutamine have been shown to function as important amino acids regulating tumor growth and survival. To assess the levels of glutamate in primary gastric cancer samples, a commercially-available colorimetric glutamate assay was used to measure glutamate levels in a panel of matched tumor and normal pairs (see Methods above). Significantly elevated levels of glutamate were detected in primary tumors compared to matched normal stomach controls (n=20, p=0.038, paired t-test) (FIG. 12A). To test the influence of CD44-SLC1A2 on intracellular glutamate, levels of intracellular glutamate across the gastric cancer cell lines was compared. A significantly higher basal glutamate level in CD44-SLC1A2 expressing SNU16 cells was observed compared to AGS cells (FIG. 12B, p=0.009). However, after CD44-SLC1A2 siRNA treatment, SNU16 glutamate levels were significantly reduced compared to scrambled siRNA controls (FIG. 12B, p=0.01). No significant effects were observed when the fusion siRNA was applied AGS cells (FIG. 12B). This observation suggests that CD44-SLC1A2 may function to regulate intracellular glutamate levels in gastric cancer.

To test if CD44-SLC1A2 silencing might sensitize gastric cancer cells to drug treatment, control and CD44-SLC1A2 silenced SNU16 cells was treated to increasing concentrations of cisplatin, a chemotherapy reagent commonly used in gastric cancer treatment, and computed GI50s, the drug concentration required to cause 50% growth inhibition. SNU16 cells were found to be significantly more sensitive to cisplatin after CD44-SLC1A2 siRNA treatment, with a reduction in GI50 (the concentration required to cause 50% growth inhibition) from 11.8 μM to 3.96 μM (p=1.11×10-6, FIG. 12C). The sensitization of CD44-SLC1A2 silenced cells appears to be specific to cisplatin, as no differences between control and silenced cells were observed upon treatment with 5-fluorouracil, another gastric cancer chemotherapy (FIG. 13).

Tumors Expressing High SLC1A2 Levels are Associated with CD44-SLC1A2 Positivity

CD44 is highly expressed in many cancers including gastric cancer. One consequence of the CD44-SLC1A2 fusion might thus be to place SLC1A2 under the regulatory control of CD44 promoter elements, causing high levels of SLC1A2 expression in tumors. If this is the case, then tumors expressing high levels of SLC1A2 should also tend to be CD44-SLC1A2 positive. To explore this possibility, a previously described gene expression database of 197 gastric cancers was analyzed to identify tumors expressing high SLC1A2 levels. Fifteen gastric cancer samples were screened from the top 15 percent of SLC1A2 overexpressing tumors for CD44-SLC1A2 expression. Among the fifteen tumors, five gastric cancer samples expressed the CD44-SLC1A2 fusion transcript (FIG. 14, blue crosses), and none of the matched adjacent normal tissues expressed CD44-SLC1A2 (FIG. 14). Thus, while the rate of CD44-SLC1A2 positively in an unselected patient cohort is low (1-2%), the CD44-SLC1A2 positively rate is markedly elevated in this selected subpopulation (33%, 5 out of 15 tumors). This result is consistent with the CD44-SLC1A2 fusion causing the transcriptional upregulation of SLC1A2. High SLC1A2 expression levels observed in CD44-SLC1A2 negative tumors may be due to alternative mechanisms, such as focal genomic amplification, fusion to other partners, and EGF or mTOR/Akt signaling.

An unsupervised clustering analysis of the 197 gastric tumors revealed that the majority of high SLC/A2-expressing tumors tended to cluster together (>75%), suggesting that high SLC1A2 expression may define a distinct molecular subgroup of gastric cancer (FIG. 15). To identify predominant biological themes associated with this molecular subgroup, gene ontology analysis was performed on a 710-gene ‘SLC1A2 signature’, generated by comparing the top 15% of SLC1A2 high-expressing tumors against the bottom 15% (Wilcoxon signed ranked test, False Discovery Rate FDR=0.005). Genes expressed in SLC1A2 high expressing tumors were associated with ribosomal biosynthesis and protein translation (corrected p=5.12×10-33; Fisher test, Table 2). These results suggest that tumors expressing high SLC1A2 levels, either through CD44 fusion or alternative mechanisms, may comprise a distinct subclass of gastric cancer.

TABLE 2
Gene Ontology Analysis of SLC1A2-High Expressing Tumors

| | | | | | |
|---|---|---|---|---|---|
| Category | Term | Count | % | PValue | Benjamini |

Gene Ontologies upregulated in SLC1A2-high Tumors

| | | | | | |
|---|---|---|---|---|---|
| GOTERM_BP_FAT | GO: 0006414~translational | 39 | 10.99 | 3.94E−39 | 5.79E−36 |
| | elongation | | | | |
| SP_PIR_KEYWORDS | ribosome | 32 | 9.01 | 1.43E−35 | 5.12E−33 |
| KEGG_PATHWAY | hsa03010:Ribosome | 34 | 9.58 | 3.80E−33 | 4.41E−31 |
| GOTERM_CC_FAT | GO: 0022626~cytosolic | 32 | 9.01 | 4.83E−32 | 1.24E−29 |
| | ribosome | | | | |
| GOTERM_MF_FAT | GO: 0003735~structural | 37 | 10.42 | 1.97E−28 | 9.38E−26 |
| | constituent of ribosome | | | | |
| SP_PIR_KEYWORDS | ribosomal protein | 38 | 10.70 | 2.29E−28 | 4.11E−26 |
| GOTERM_CC_FAT | GO: 0033279~ribosomal | 33 | 9.30 | 2.41E−26 | 3.08E−24 |
| | subunit | | | | |
| GOTERM_CC_FAT | GO: 0005840~ribosome | 39 | 10.99 | 3.07E−25 | 2.62E−23 |
| GOTERM_CC_FAT | GO: 0044445~cytosolic part | 34 | 9.58 | 5.23E−25 | 3.35E−23 |
| SP_PIR_KEYWORDS | ribonucleoprotein | 40 | 11.27 | 4.39E−24 | 5.26E−22 |

Gene Ontologies downregulated in SLC1A2-high Tumors

| | | | | | |
|---|---|---|---|---|---|
| SP_PIR_KEYWORDS | acetylation | 49 | 39.84 | 1.19E−12 | 2.76E−10 |
| SP_PIR_KEYWORDS | phosphoprotein | 75 | 60.98 | 1.45E−07 | 1.67E−05 |
| GOTERM_CC_FAT | GO: 0043233~organelle | 28 | 22.76 | 7.16E−05 | 8.66E−03 |
| | lumen | | | | |
| GOTERM_CC_FAT | GO: 0031974~membrane- | 29 | 23.58 | 3.65E−05 | 8.84E−03 |
| | enclosed lumen | | | | |
| GOTERM_CC_FAT | GO: 0070013~intracellular | 27 | 21.95 | 1.32E−04 | 1.06E−02 |
| | organelle lumen | | | | |
| GOTERM_CC_FAT | GO: 0031981~nuclear lumen | 23 | 18.70 | 3.01E−04 | 1.81E−02 |
| UP_SEQ_FEATURE | short sequence motif:Nuclear | 11 | 8.94 | 4.20E−05 | 2.43E−02 |
| | localization signal | | | | |
| SP_PIR_KEYWORDS | repressor | 11 | 8.94 | 4.28E−04 | 3.24E−02 |

Category - Database;
Term - Enriched ontology term;
Count - Number of genes overlapping with the SLC1A2 signature;
% - percentage of overlapping genes from input signature list;
Pvalue - Modified Fisher exact test pvalue;
Benjamini - corrected p value using the Benjamini method

CD44-SLC1A2 Expression can Occur Independently of 11p13 Amplification

CD44-SLC1A2 was initially identified through tumors exhibiting 11p13 genomic amplification (FIG. 3B). However, 11p13 amplification may not be an absolute prerequisite for CD44-SLC1A2 fusion expression. To investigate the relationship between 11p13 genomic amplification and CD44-SLC1A2 expression, seven fusion positive tumors was analyzed using Affymetrix SNP6 arrays. Of seven fusion-positive tumors, two tumors (GC980390 and GC990172) exhibited evidence of 11p13 genomic amplification, while the other five did not (FIG. 16A1-A2). This finding demonstrates that CD44-SLC1A2 expression can be observed in tumors independently of 11p13 genomic amplification. Further supporting the notion that 11p13 amplification and CD44-SLC1A2 gene fusion are distinct events, a comparison of CD44 and SLC1A2 expression levels across 45 gastric tumors, including a) 11p13 non-amplified samples (32 samples); b) 11p13 amplified but fusion-negative samples (6 samples), and c) CD44-SLC1A2 fusion positive samples (7 samples) revealed that high SLC1A2 expression levels may be more strongly associated with fusion-events rather than generalized 11p13 amplification (FIG. 17).

### Impact of 11p13 Amplifications and CD44-SLC1A2 Fusions on SLC1A2 and CD44 Expression

CD44 and SLC1A2 expression levels were compared across 45 gastric tumors, including a) 11p13 non-amplified samples (32 samples); b) 11p13 amplified but fusion negative samples (6 samples), and c) CD44-SLC1A2 fusion positive samples (7 samples). The rate of 11p13 amplification in this series (˜17%) is similar to frequencies previously reported in the literature. It is important to note that in this experiment, the expression measurements were inferred using U133P2 Affymetrix microarray probes, which target the 3′ ends of genes. Compared to non-amplified samples, fusion positive samples exhibited significantly increased 3′ SLC1A2 gene expression (P=0.004), but 11p13 amplified samples did not (P=0.86) (FIG. 17A). These findings suggest that high SLC1A2 expression levels may be driven more by fusion-events rather than generalized 11p13 amplification The one exception was a sample with a high-level focal 11p13 amplification (GC980417)—in this tumor, SLC1A2 was highly expressed (FIG. 17A). Intriguingly, unlike SLC1A2, a very different scenario was observed for CD44. Specifically, while CD44 3′ transcripts were significantly overexpressed in 11p13 amplified tumors (p=0.016), they were significantly underexpressed in fusion positive tumors (p=0.006) (FIG. 17B). The inventors speculate that this latter finding may be due to the CD44/SLC1A2 genomic inversion decoupling the 3′ end of the CD44 gene (the region detected by the Affymetrix array) from the endogenous CD44 promoter. Additional evidence of this decoupling was obtained in a real-time PCR analysis measuring SLC1A2 exon 1, where unlike the 3′ SLC1A2 transcripts, SLC1A2 exon 1 (which is not part of the CD44-SLC1A2 fusion) was not observed to be highly expressed relative to non-amplified samples in fusion positive samples (FIG. 17C-D).

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

### Survival Analysis

From a large cohort of 197 primary tumors from which both gene expression and clinical information were available, high SLC1A2 expressing tumors was analyzed for any potential association with any distinct clinicopathologic traits (for example, age, gender, pathologic grade or histology by Lauren's classification). High expression of SLC1A2 was determined using a cut-off of the top 15th percentile of SLC1A2 expression. High expression of SLC1A2 was not related to any of the clinicopathological traits discussed above. However, overexpression of SLC1A2 was related to early stage disease (p=0.01), i.e. stage I/II of gastrointestinal cancer. High SLC1A2 expression was prognostic for improved survival in univariate analysis (median overall survival: 113 vs 23 months, HR: 0.41, 95% CI: 0.22-0.79, p=0.007) (FIG. 4C) and bivariate analysis after accounting for a possible confounder of disease stage (HR 0.48, 95% CI: 0.25-0.92, p=0.03). Similar results were obtained when the SLC1A2 percentile cut-off was varied by +/−5%.

1. A method of treating or preventing a gastrointestinal cancer, comprising:
inhibiting expression of a CD44-SLC1A2 fusion gene or a functional variant thereof, or
inhibiting the activity of a fusion protein encoded by the CD44-SLC1A2 fusion gene or the functional variant thereof.

2. The method of claim 1, wherein preventing comprises preventing development of a gastrointestinal cancer in a patient who was not diagnosed with a gastrointestinal cancer before or preventing relapse of a gastrointestinal cancer in a patient who was previously diagnosed with a gastrointestinal cancer.

3. The method of claim 1, wherein the gastrointestinal cancer is selected from among cancer of the gastrointestinal tract, liver cancer, cancer of the biliary tract, pancreas cancer and cancer of the peritoneum.

4. The method of claim 3, wherein:
the cancer of the gastrointestinal tract is selected from the group consisting of cancer of the upper gastrointestinal tract, cancer of the lower gastrointestinal tract, and gastrointestinal stromal tumor; or
the cancer of the upper gastrointestinal tract is selected among esophageal cancer and gastric (stomach) cancer; or
the cancer of the lower gastrointestinal tract is selected from the group consisting of cancer of the small intestine, cancer of the appendix, cancer of the colon or rectum, and cancer of the anus.

5. The method of claim 4, wherein inhibition of expression of the gene comprises administering at least one oligonucleotide.

6. The method of claim 5, wherein the oligonucleotide is an RNA silencing agent.

7. The method of claim 6, wherein the RNA silencing agent is an interfering ribonucleic acid.

8. The method of claim 7, wherein the interfering ribonucleic acid is a siRNA or shRNA or miRNA.

9. The method of claim 8, wherein:
the siRNA is directed against the nucleic acid transcribed from the SLC1A2 gene; and
the CD44-SLC1A2 fusion gene comprises the nucleotide sequence set forth as SEQ ID NO: 27 or SEQ ID NO: 28.

10. A method of sensitizing cells to a cancer treatment in a patient suffering from a gastrointestinal cancer, comprising:
inhibiting expression of a SLC1A2 gene, or a CD44-SLC1A2 fusion gene, or a functional variant of these genes; or
inhibiting the activity of a fusion protein encoded by the CD44-SLC1A2 fusion gene or its functional variant, or the protein encoded by the SLC1A2 gene or its functional variant.

11. The method of claim 10, wherein the gastrointestinal cancer is selected from among cancer of the gastrointestinal tract, liver cancer, cancer of the biliary tract, pancreas cancer and cancer of the peritoneum.

12. The method of claim 11, wherein:
the cancer of the gastrointestinal tract is selected from the group consisting of cancer of the upper gastrointestinal tract, cancer of the lower gastrointestinal tract, and gastrointestinal stromal tumor; or
the cancer of the upper gastrointestinal tract is selected among esophageal cancer and gastric (stomach) cancer; or
the cancer of the lower gastrointestinal tract is selected from the group consisting of cancer of the small intestine, cancer of the appendix, cancer of the colon or rectum, and cancer of the anus.

13. The method of claim 12, wherein inhibition of expression of the gene comprises administering at least one oligonucleotide.

14. The method of claim 13, wherein the oligonucleotide is an RNA silencing agent.

15. The method of claim 14, wherein the RNA silencing agent is an interfering ribonucleic acid.

16. The method of claim 15, wherein the interfering ribonucleic acid is a siRNA or shRNA or miRNA.

17. The method of claim 16, wherein:
the siRNA is directed against the nucleic acid transcribed from the SLC1A2 gene; and
the CD44-SLC1A2 fusion gene comprises the nucleotide sequence set forth as SEQ ID NO: 27 or SEQ ID NO: 28.

18. The method of claim 10, wherein the cancer treatment comprises administration of at least one chemotherapeutic agent.

19. The method of claim 18, wherein the cancer treatment is a chemotherapy.

20. The method of claim 18, wherein the chemotherapeutic agent is selected from among alkylating agents, antimetabolites, antimitotics, topoisomerase inhibitors, platinum derivatives, hormonal therapies, signaling inhibitors, biological response modifiers and differentiating agents; provided that the chemotherapeutic agent 5-fluorouracil and a prodrug thereof is excluded.

21. The method of claim 18, wherein the chemotherapeutic agent is selected from among cisplatin, adriamycin (doxorubicin), nutlin-3, and etoposide phosphate.

22. A method of reducing intracellular glutamate level of a cell, comprising:
inhibiting expression of a CD44-SLC1A2 fusion gene or a functional variant thereof, or
inhibiting the activity of a fusion protein encoded by the CD44-SLC1A2 fusion gene or the functional variant thereof.

23. The method of claim 22, wherein the gastrointestinal cancer is selected from among cancer of the gastrointestinal tract, liver cancer, cancer of the biliary tract, pancreas cancer and cancer of the peritoneum.

24. The method of claim 23, wherein:
the cancer of the gastrointestinal tract is selected from the group consisting of cancer of the upper gastrointestinal tract, cancer of the lower gastrointestinal tract, and gastrointestinal stromal tumor; or
the cancer of the upper gastrointestinal tract is selected among esophageal cancer and gastric (stomach) cancer; or
the cancer of the lower gastrointestinal tract is selected from the group consisting of cancer of the small intestine, cancer of the appendix, cancer of the colon or rectum, and cancer of the anus.

25. The method of claim 22, wherein inhibition of expression of the gene comprises administering at least one oligonucleotide.

26. The method of claim 25, wherein the oligonucleotide is an RNA silencing agent.

27. The method of claim 26, wherein the RNA silencing agent is an interfering ribonucleic acid.

28. The method of claim 27, wherein the interfering ribonucleic acid is a siRNA or shRNA or miRNA.

29. The method of claim 28, wherein:
the siRNA is directed against the nucleic acid transcribed from the SLC1A2 gene; and the CD44-SLC1A2 fusion gene comprises the nucleotide sequence set forth as SEQ ID NO: 27 or SEQ ID NO: 28.

30.-50. (canceled)
